(19) United States
(12) Patent Application Publication
(10) Pub. No.: US 2026/0308535 A1
(43) Pub. Date: Oct. 8, 2026

(54) ANTI-CANCER TREATMENTS AND METHODS OF USE THEREOF

(71) Applicants: Memorial Sloan-Kettering Cancer Center, New York, NY (US); Memorial Hospital for Cancer and Allied Diseases, New York, NY (US); Sloan-Kettering Institute for Cancer Research, New York, NY (US)

(72) Inventors: Andrew M. INTLEKOFER, New York, NY (US); Shengqi HOU, New York, NY (US)

(21) Appl. No.: 19/480,474

(22) PCT Filed: May 3, 2024
(86) PCT No.: PCT/US2024/027622

§ 371 Date: Oct. 31, 2025

Related U.S. Application Data
(60) Provisional application No. 63/463,871, filed on May 3, 2023.

(51) Int. Cl. C12N 9/04; A61K 31/4375; A61K 31/455; A61K 31/7068; A61K 38/00; A61K 48/00; A61P 35/02; C12N 9/22; C12N 15/11; C12N 15/86; C12Q 1/26; G01N 33/50

(52) U.S. Cl. CPC C12N 9/0006; A61K 31/4375; A61K 31/455; A61K 31/7068; A61K 48/005; A61P 35/02; C12N 9/222; C12N 15/111; C12N 15/86; C12Q 1/26; G01N 33/5023; A61K 38/00; C12N 2310/20; C12N 2740/10043; C12N 2740/15043; C12Y 101/01042; G01N 2333/902

(57) ABSTRACT

This invention is directed to anti-cancer treatments and methods of use thereof.

### CROSS-REFERENCE TO RELATED APPLICATIONS

This application claims the benefit of and priority to U.S. Provisional Application No. 63/463,871, filed on May 3, 2023, the entire contents of which are incorporated herein by reference.

### GOVERNMENT INTEREST

This invention was made with government support under CA251543, CA254838 and CA008748 awarded by the National Institutes of Health. The government has certain rights in the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### FIELD OF THE INVENTION

This invention is directed to anti-cancer treatments and methods of use thereof.

### BACKGROUND OF THE INVENTION

Oncogenic gain-of-function mutations in the active site of isocitrate dehydrogenase 1 and 2 (IDH1 and IDH2) allows production of the metabolite 2-hydroxyglutarate (2HG), which drives oncogenesis through pleiotropic effects on chromatin, metabolism, and differentiation. Allosteric inhibitors of mutant IDH enzymes bind to the IDH dimer interface, block production of 2HG, and induce differentiation of IDH-mutant cancer cells. However, despite suppressing 2HG production in IDH-mutant acute myeloid leukemias, mutant IDH inhibitors induce clinical responses in less than half of cases.

### SUMMARY OF THE INVENTION

Disclosed herein are methods for treating a cancer in a subject by hyperactivating an oncogenic activity of an enzyme in cancer cells. In some embodiments, the enzyme can be a mutated endogenous enzyme. The endogenous enzyme can acquire oncogenic activity because of the mutation(s). In some embodiments, the enzyme can be a mutant isocitrate dehydrogenase (mutIDH). The mutIDH can be a mutIDH1 or IDH2. In some embodiments, the mutIDH can catalyze reaction of α-ketoglutarate (AKG) to 2-hydroxyglutarate (2HG). In some embodiments, the mutIDH can be resistant to a mutIDH inhibitor. In the method, hyperactivating the oncogenic activity of a mutIDH can be toxic to the cancer cells.

In some embodiments, treating the cancer to hyperactivate an oncogenic activity of an enzyme can be through use of a molecule(s) that hyperactivates the oncogenic activity. In some embodiments, the molecule can increase an amount of 2HG in cells of the cancer. In some embodiments, the molecule can activate/hyperactivate the mutIDH in cells of the cancer. In some embodiments, the molecule can facilitate the mutIDH to modify its cofactor preference. In some embodiments, the molecule can be a small molecule. The molecule may bind to a hydrophobic pocket at a dimer-interface of a mutIDH enzyme. In some embodiments, the molecule can interact with one or more amino acid residues in mutIDH. In mutIDH2, the amino acid residues can be S200, K243, W244, P245, Y247, K282, W284, R288, M293, Q296, S301, G303, F304, and W306, or homologous amino acid residues in mutIDH1. In some embodiments, the molecule can move A and B subunits of mutIDH proximally closer to one another. The molecule may strengthen interactions across/within an interface of two mutIDH subunits. In some embodiments, the molecule can be clonixin, idebenone, dihydrogedunin, deoxygedunin, gossypetin, derivatives thereof and/or combinations thereof. Other molecules are disclosed herein.

In some embodiments, disclosed are use of compounds that can hyperactivate a mutant isocitrate dehydrogenase (mutIDH) to treat a subject having a cancer that has a mutIDH. Cells of the cancer can have 2HG. The compounds that can hyperactivate the mutIDH can include clonixin, EDBN, derivatives thereof and related compounds. The cancer cells can be resistant to an allosteric mutIDH inhibitor.

In an embodiment, disclosed are use of molecules that hyperactivate mutTDH (e.g., clonixin, EDBN) for manufacture of a medicament to treat a subject having a cancer that has a mutIDH. The mutIDH can catalyze reaction of α-ketoglutarate to 2-hydroxyglutarate (2HG). The cancer can be resistant to a mutIDH inhibitor.

Other objects and advantages of this invention will become readily apparent from the ensuing description.

### BRIEF DESCRIPTION OF THE FIGURES

FIGS. 1A-C, 1D-I and J-N show non-limiting, representative data of in cis dimer-interface mutation in IDH2 causes metabolic dysfunction and impairs cell growth. Panel A Schematic depicting IDH2 dimers retrovirally transduced into Ba/F3 cells. ‘Pre’received IDH2 WT-mCherry plus IDH2 RQ-YFP; ‘Trans’ received IDH2 RQ-YFP plus IDH2 IM-GFP; ‘Cis’ received IDH2 WT-mCherry plus IDH2 RQ/IM-GFP. Panel B Overlay (left) of sorted cells indicating the ability to distinguish populations by distinct fluorescent markers. Western blot (right) showing IDH2 expression and vinculin loading control. Panel C Competitive growth of ‘Pre’, ‘Trans’, and ‘Cis’ cells mixed at a 1:1:1 ratio and cultured with vehicle (DMSO) or the mutant IDH2 inhibitor enasidenib (100 nM). Changes in the percentage of ‘Pre’, ‘Trans’, and ‘Cis’ cells were assessed weekly by flow cytometry (n=3). Data are mean±95% confidence intervals. Panel D Oxygen consumption rate (OCR) as measured using the Seahorse bioanalyzer. Values are mean±SD (n=5). Panel E Mitochondrial membrane potential (ΔΨm) of ‘Pre’, ‘Trans’, and ‘Cis’ cells stained with MitoProbe™ DilC1(5). MitoTracker Deep Red FM was used to determine mitochondrial content in ‘Pre’, ‘Trans’, and ‘Cis’ cells (see FIG. 14 panel d). Panel F Schematic depicting IDH2 dimers introduced into Ba/F3 cells, harboring the active-site R140Q mutation alone (‘RQ’) or the active-site R140Q mutation in cis with the I319M dimer-interface mutation (‘RQ/IM’). Panel G Western blot showing inducible IDH2 expression in Ba/F3 cells with (+) or without (−) doxycycline treatment for 48 hr; vinculin is the loading control. Panel H Proliferation of Ba/F3 cells cultured with (+) or without (−) doxycycline (n=3). Panel I Intracellular 2HG levels from Ba/F3 cells cultured with doxycycline for 48 hr (n=3). Panel J Basal oxygen consumption rate (OCR) in Ba/F3 cells with doxycycline-induced expression of RQ or RQ/IM (n=5). Panel K Mitochondrial superoxide measured by the MitoSOX probe (n=4). Panel L Heatmap representing steady-state levels of metabolites as measured by GC-MS in Ba/F3 cells with doxycycline-induced expression of RQ or RQ/IM (normalized to RQ average). Panel M Lactate/Pyruvate ratio in the media of Ba/F3 cells with doxycycline-induced expression of RQ or RQ/IM (normalized to RQ) after 48 hr of culture. Panel N NADH/NAD⁻ ratio (Glo assay) from Ba/F3 cells with doxycycline-induced expression of RQ or RQ/IM (n=3). Results throughout figure are representative of >3 independent experiments. Data are mean±SEM unless otherwise specified. **P<0.01, ***P<0.001, ****P<0.0001.

FIGS. 2 A-E and F-K show non-limiting, exemplary data indicating in cis dimer-interface mutation hyperactivates IDH2 and facilitates NADH-dependent 2HG production. Panel A Schematic of IDH2 heterodimers harboring the active-site R140Q and dimer-interface I319M mutations in Trans or Cis. Right, relative velocity of NADPH-dependent 2HG production compared to IDH2 R140Q heterodimers. Reactions were performed with purified enzymes (7.5 μg/ml), NADPH (0.33 mM), and αKG (as indicated) (n=3). Panel B Schematic of IDH2 homodimers with RQ or RQ/IM mutations. Right, rate of NADPH-dependent 2HG production as monitored by Abs 340 nm. Reactions were performed with purified enzyme (7.5 μg/ml), NADPH (0.33 mM), and αKG (1.0 mM) (n=3). Panel C Absolute velocity of NADPH− dependent 2HG production for RQ or RQ/IM homodimers shown in b. Reactions were performed with purified enzyme (7.5 μg/ml), NADPH (0.33 mM), and αKG (0.5, 1.0, 2.0, 5.0 mM) (n=3). Panel D Rate of NADH-dependent 2HG production by purified IDH2 homodimers as monitored by Abs 340 nm. Reactions were performed with purified enzyme (7.5 μg/ml), NADH (0.25 mM), and αKG (5.0 mM) (n=3). Panel E Rate of NAD*-dependent isocitrate oxidation by purified IDH2 homodimers as monitored by Abs 340 nm. Reactions were performed with purified enzyme (7.5 μg/ml), NAD⁺ (3.3 mM), and isocitrate (5.0 mM) (n=3). Panel F Ribbon and surface representation of the IDH2 R140Q ternary dimer showing subunit A (salmon) and B (grey) at 1.75 Å resolution. Relevant features shown include I319 (salmon/grey), Q140 (magenta), αKG (pink), NADP⁺ (black), and Mg²⁺ (green). Panel G Ribbon and surface representation of the IDH2 R140Q/I319M ternary dimer showing subunit A (green) and B (blue) at 1.81 Å resolution. Relevant features shown as in (Panel F) except for M319 (magenta). Panel H Close-up view of IDH2 R140Q (FIG. 2 panel F) showing residues that are involved in π-π and S-π interactions (black dash-lines) at the dimer interface to which I319 does not contribute. For the sake of clarity, dash-lines are predominantly shown for subunit A. Panel I Close-up view of IDH2 R140Q/I319M (FIG. 2 panel G) showing residues that are involved in π-π and S-π interactions (red dash-lines) at the dimer interface to which M319 contributes. Panel J Hydrophobic interactions of two I319 with surrounding residues at the dimer interface of IDH2 R140Q. Side chains of involved residues are depicted by surface and stick-model representations. Panel K S-π and hydrophobic interactions of two M319 with surrounding residues at the dimer interface of IDH2 R140Q/I319M. Side chains of involved residues are depicted by surface and stick-model representations. Values for enzyme reactions are mean±95% confidence intervals. ns, not significant (P>0.05), *P<0.05, ****P<0.0001.

FIGS. 3A-F and G-M show non-limiting, representative data indicating toxic activation of mutant IDH2 by a small molecule. Panel A: Schematic of chemical screen to identify small molecules that facilitate NADH-dependent 2HG production by purified IDH2 R140Q enzyme. Panel B: Clonixin was identified as a hit (Z-score>=6) from the Spectrum MicroSource Library of 2560 compounds. Figure shows one of two independent screens. See also Table 3. Panel C: Clonixin facilitates NADH-dependent 2HG production as assessed by velocity of NADH consumption. Reactions included purified IDH2 R140Q enzyme (7.5 μg/ml), NADH (0.25 mM), αKG (5.0 mM), and vehicle (DMSO) or clonixin (25 μM) (n=3). Panel D: NADH-dependent 2HG production with clonixin. Enzyme reactions as in c were collected after 3 hr and 2HG was measured by GC-MS (n=3). Panels E, F: Clonixin enhances NADPH-dependent 2HG production. Panel E: Rate of NADPH consumption and (Panel F) absolute velocity of NADPH-dependent 2HG production. Reactions included purified enzyme (7.5 μg/ml), NADPH (0.33 mM), and αKG [1.0 mM (e); 0.5, 1.0, 2.0, 5.0 mM (f)](n=3). Panel G: Clonixin binds to periphery of dimer-interface. Cartoon and surface representation of the crystal structure (2.5 Å resolution) of IDH2 R140Q dimer (subunit A and B in purple and orange, respectively) with NADP⁺ and clonixin. The substrate αKG, Mg²⁺, coordinated to two water molecules in each subunit are from the structure of IDH2 R140Q in FIG. 2 Panel F. Panel H: Close-up view of clonixin bound to a hydrophobic pocket. Residues interacting with clonixin (chocolate) bound to Subunit A (purple) are shown with stick models and labeled. The cofactor NADP⁺ (black for C atoms) bound to subunit B (orange) and R353 are depicted with stick models. The Fo-Fc omit map contoured at 3σ for clonixin is depicted in density mesh. The red dash lines represent hydrogen bonds. Panel I: Close-up view of clonixin and several residues in stick models from subunit A (purple) and B (orange) after superposition of IDH2 R140Q dimer bound to clonixin and the R140Q dimer (salmon/grey) shown in FIG. 2 panel F. The Fo-Fc omit map contoured at 3σ for R288 (purple) and R353 (orange) from respective subunits A and B in the clonixin-bound structure is also shown in density mesh. Panel J: Selective toxicity of clonixin toward IDH2-mutant cells. Ba/F3 cells transduced with IDH2 WT or R140Q were subjected to vehicle or clonixin (350 or 400 μM) for 2 days. Cell death (DAPI⁺) was measured by flow cytometry. Panels K, L, and M: Clonixin selectively eliminates IDH2-mutant leukemia cells from competitive growth cultures. Panel K Ba/F3 cells expressing IDH2 WT (mCherry) or R140Q (GFP) (n=4), Panel L human TF1 cells isogenic for IDH2 WT (mCherry) or R140Q (GFP) in endogenous locus (n=5), or Panel M MLL-AF9-transduced primary HSPCs from LSL-Idh2^{R140Q} mice with (‘RQ’; GFP/mCherry) or without (‘WT’; GFP) Cre transduction (n=5). Cells were mixed at 1:1 ratio then cultured with vehicle (DMSO) or clonixin (200 μM). Populations were assessed by flow cytometry at the indicated timepoints (n=5). Data for (panel c), (panel d), (panel f), (panel j) are mean±SEM; data for (panel e), (panel k), (panel 1), (panel m) are mean±95% CI. **P<0.01, ***P<0.001, ****P<0.0001.

FIG. 4A-D shows non-limiting, exemplary data indicating in cis dimer-interface mutation impairs leukemia cell growth. Panel A: Full membrane with Western blot showing IDH2 expression and vinculin loading control in Ba/F3 cells stably transduced with ‘EV (empty vector)’, ‘Pre’, ‘Trans’, and ‘Cis’ allelic configurations described in FIG. 1 panels J and K. Red outline denotes the area shown in FIG. 1 panel B. Panels B and C: ‘Pre’, ‘Trans’, and ‘Cis’ cells were mixed at ratios of 3:3:4 (Panel B) or 1:1:2 (Panel C) and cultured with vehicle (DMSO) or enasidenib (100 nM). Changes in the percentage of ‘Pre’, ‘Trans’, and ‘Cis’ cells were assessed weekly by flow cytometry (n=3). Panel D: Representative histograms of MitoTracker Deep Red FM expression (left), and the median fluorescence intensity of (MFI) of MitoTracker Deep Red FM in in ‘Pre’, ‘Trans’, and ‘Cis’ cells (right), (n=3). Data for Panel B and C are mean±95% confidence intervals for triplicate samples. Data for panel D are mean±SEM for triplicate samples.

FIGS. 5A-C, D-F and G-I show non-limiting, exemplary data of purification and activity of IDH2 heterodimers. Panel A: Schematic of experimental approach: 293T cells were co-transfected with HA-tagged IDH2 R140Q plus FLAG-tagged wildtype or I319M (in trans), or HA-tagged IDH2 wildtype plus FLAG-tagged wildtype, R140Q, I319M or R140Q/I319M (in cis). After 2 days, cells were lysed and enzyme complexes were purified by HA-affinity resin. Reactions were performed with purified enzyme (7.5 μg/ml), NADPH (0.33 mM) or NADP⁺ (1.0 mM), and αKG or isocitrate at the indicated concentrations. Panels B and C: HA-purified enzymes were assessed by denatured SDS-PAGE (Panel B), or native PAGE with Coomassie blue staining (panel C). Panels D, E, and F: Schematic of IDH2 heterodimers with R140Q and wildtype or I319M mutations in trans (panel D). NADPH-dependent 2HG production (panel E) and NADP⁺-dependent isocitrate oxidation (panel F) by IDH2 heterodimers purified as in panel B and panel C and monitored by Abs 340 nm. Reactions were performed with purified enzyme (7.5 μg/ml), NADPH (0.33 mM) or NADP⁺ (1.0 mM), and αKG or isocitrate at the indicated concentrations (n=3). Panels G, H, and I: Schematic of IDH2 heterodimers with wild-type and wildtype, R140Q, I319M or R140Q/I319M in cis (panel G). NADPH-dependent 2HG production (panel H) and NADP⁺-dependent isocitrate oxidation (Panel I) by IDH2 heterodimers purified as in panels B and C and monitored by Abs 340 nm. Reactions were performed with purified enzyme (7.5 μg/ml), NADPH (0.33 mM), NADP⁺ (1.0 mM), and αKG or isocitrate at the indicated concentrations (n=3). Value throughout are mean±95% confidence intervals for triplicate reactions.

FIGS. 6A-C, D-F and G-I show non-limiting, exemplary data of purification and activity of IDH2 homodimers. Panel A: Schematic of experimental approach: 293T cells were transfected with FLAG-tagged IDH2 wildtype, R140Q, I319M or R140Q/I319M. After 2 days, cells were lysed and enzyme complexes were purified by FLAG-affinity resin. Reactions were performed with purified enzymes (7.5 μg/ml), NADPH (0.33 mM) or NADP⁺ (1.0 mM), and αKG or isocitrate at the indicated concentrations. Panels B and C: FLAG-purified enzymes were assessed by denatured SDS-PAGE (panel B) or native PAGE with Coomassie blue staining (panel C). Panels D, E, and F: Schematic of IDH2 homodimers of wildtype, R140Q, I319M or R140Q/I319M (panel D). NADPH-dependent 2HG production (panel E) and NADP⁺-dependent isocitrate oxidation (panel F) by IDH2 homodimers purified as in panels B and C and monitored by Abs 340 nm. Reactions were performed with purified enzyme (7.5 g/ml), NADPH (0.33 mM) or NADP⁺ (1.0 mM), and αKG or isocitrate at the indicated concentrations (n=3). Panels G and H: Kinetic parameters of IDH2 RQ and RQ/IM homodimers. Kₘ and Vₘₐₓ values were calculated using the Michaelis-Menten equation in GraphPad Prism. Panel I: Velocity of NADH-dependent 2HG production as a function of αKG concentration for IDH2 RQ and RQ/IM homodimers. Data for panels E and G are mean±95% CI; data for panels G, H, and I are mean±SEM.

FIGS. 7A-C, D and E-F show non-limiting, exemplary data of purification and activity of IDH2 homodimers from E. coli. Panel A: His-tagged IDH2 R140Q or R140Q/I319M (in cis) protein were purified using Nickel-charged nitrilotriacetic acid (NTA) agarose resin and assessed by denatured SDS-PAGE with Coomassie blue staining. Panels B and C: NTA-purified His-tagged IDH2 enzymes were further purified by size exclusion chromatography and assessed by denatured SDS-PAGE with Coomassie blue staining (Panel B), Native PAGE with Coomassie blue staining (Panel C). Panel D: In vitro enzyme assay measuring rate of NADPH-dependent 2HG production of IDH2 dimers purified as in panels A to C. Reactions were performed with purified enzyme (7.5 μg/ml), NADPH (0.33 mM), and αKG at the indicated concentrations (n=3). Panels E and F: Rate of NAD⁺-dependent isocitrate oxidation panel E and NADH-dependent 2HG production panel F by purified IDH2 homodimers as monitored by Abs 340 nm. Reactions were performed with purified enzyme (7.5 μg/ml), NAD⁺ (3.3 mM) or NADH (0.25 mM), and αKG or isocitrate at indicated concentrations (n=3). Data are mean±95% confidence intervals for triplicate reactions.

FIG. 8A-E shows exemplary structures of human IDH2 R140Q and R140Q/I319M with NADP⁺, Mg²⁺, αKG, and clonixin. Panel A: The Fo-Fc omit map contoured at 3σ for NADP⁺ (black), Mg²⁺ (dark green sphere), αKG (pink), and water molecules (red solid spheres) are depicted in purple mesh for the IDH2 RQ ternary crystal structure from crystallization condition #1 (Table 2). Panel B: The Fo-Fc omit map contoured at 3σ for NADP⁺ (black), Mg²⁺ (dark green sphere), αKG (pink), and water molecules (red solid spheres) are depicted in purple mesh for the IDH2 RQ/IM ternary crystal structure from crystallization condition #1 (Table 2). Panel C: The Fo-Fc omit map contoured at 3σ for R288 (salmon) and R353 (grey) at the dimer-interface of IDH2 RQ ternary structure. Panel D: The Fo-Fc omit map contoured at 3σ for R288 (green) and R353 (cyan) at the dimer-interface of IDH2 RQ/IM ternary structure. Panel E: The Fo-Fc omit map contoured at 3σ for R288 (purple) and R353 (orange) at the dimer-interface of IDH2 RQ bound to clonixin. Crystal structure figures were produced using PyMOL (pymol.org/2/).

FIG. 9A-H shows non-limiting, exemplary data indicating the effects of clonixin on IDH enzymes. Panel A: Schematic of IDH2 WT:RQ heterodimer. Panels B and C: NADH-dependent 2HG production panel B and NADPH-dependent 2HG production panel C by IDH2 WT:RQ heterodimers purified from bacteria with reaction monitoring by Abs 340 nm. Reactions were performed with purified enzyme (7.5 μg/ml), NADPH (0.33 mM) or NADH (0.25 mM), αKG (5.0 mM), and vehicle (DMSO) or clonixin (250 μM) (n=3). Panel D: Schematic of IDH2 WT:WT homodimer. Panels E and F: NADPH-dependent 2HG production Panel E and NADP⁺-dependent isocitrate oxidation Panel F by IDH2 WT:WT homodimers purified from bacteria with reaction monitoring by Abs 340 nm. Reactions were performed with purified enzyme (7.5 μg/ml), NADPH (0.33 mM) or NADP⁺ (1.0 mM), αKG (2.0 mM) or isocitrate (‘Iso’; 2.0 mM), and vehicle (DMSO) or clonixin (25 μM) (n=3). Panel G: Schematic of IDH1 R132C:R132C homodimer. Panel H: NADPH-dependent 2HG production by IDH1 RC:RC homodimers purified from bacteria enzymes with monitored by Abs 340 nm. Reactions were performed with purified enzyme (7.5 μg/ml), NADPH (0.33 mM), αKG (0.5 mM), and vehicle (DMSO) or clonixin (250 μM) (n=3). Data are mean±95% confidence intervals for triplicate reactions.

FIG. 10A-B shows non-limiting, exemplary data indicating the cre-mediated expression of Idh2 R140Q in primary hematopoietic cells. Panel A: Flow cytometry gating and sorting strategy to distinguish populations by distinct fluorescent markers. Primary bone marrow HSPCs from LSL-Idh2^{R140Q+/-} knock-in mice²¹ were transduced with retrovirus encoding MLL/AF9-GFP. GFP⁺ cells were sorted and transduced with retrovirus encoding Cre-mCherry to activate expression of Idh2 R140Q. Panel B: Extracellular and intracellular 2HG levels from purified populations of transduced primary HSPCs as shown in panel A (n=3). Data are mean±SEM for triplicate reactions. ****P<0.0001.

FIG. 11 shows an exemplary schematic comparing existing strategies and those described herein for therapeutic targeting of IDH2-mutant malignancies.

FIG. 12 shows a schematic of metabolic pathways of alpha ketoglutarate (αKG) and exemplary cellular outcomes. These pathways can control cell fate and oncogenesis.

FIG. 13A-B shows exemplary illustrations of cancer associated IDH mutations. These IDH mutations can cluster in the enzyme active site. Panel A: Illustration of IDH2 and IDH1 mutations. Panel B: Schematics of active sites of cytosolic IDH1 and mitochondrial IDH2 as well and structural models of the active sites of crystalized IDH1 with isocitrate and crystalized IDH2 with isocitrate (models of crystallized structures adapted from Ward et al., Cancer Cell. 2010 Mar. 16; 17(3): 225-234).

FIG. 14 shows a non-limiting, exemplary schematic indicating how IDH1 mutants can produce ‘oncometabolite’ 2-hydroxyglutarate (2HG) (adapted from Shih and Levine, Cancer Cell. 2012 Sep. 11; 22(3):285-7. doi: 10.1016/j.ccr.2012.08.022. PMID: 22975371. 11; 22). The net effect can be repressive chromatin methylation which can lead to impaired differentiation.

FIG. 15 shows an exemplary schematic of 2HG locking IDH-mutant cells in a stem cell-like state.

FIG. 16 shows schematics of drug approaches that inhibit mutant IDH enzymes.

FIG. 17A-C shows illustrations of mutant IDH inhibitor development. Panel A: Illustration of IDH2. Active site inhibitors were not found. Panel B: Timeline of discovery of IDH mutations until FDA-approvals of IDH inhibitors. Panel C: Illustration of how allosteric binding of AG-221 can stabilize inhibitory open conformation of the IDH2^{R140Q} active site (adapted from Yen et al., Cancer Discov. 2017 May; 7(5):478-493). Also see, Amatangelo et al., Blood. 2017 Aug. 10; 130(6):732-741, Stein et al., Blood. 2017 Aug. 10; 130(6):722-731, and DeNardo N Engl J Med 2018; 378:2386-2398.

FIG. 18 shows a non-limiting, exemplary schematic of IDH-mutant tumors resisting inhibitors and restoring 2HG. Patients treated with IDH inhibitors had 2HG levels in blood tracked.

FIG. 19 shows illustrations of IDH dimers adapted from Intlekofer et al, Nature. 2018 July; 559(7712):125-129. Resistance mutations can occur at IDH dimer interface where drugs bind. Secondary mutations can occur in key residues for drug binding: Q316-hydrogen bonds from amino and carbonyl side chain and carbonyl back bone; I319-van der Waals interactions.

FIG. 20A-D shows schematics of drug resistance mutations in oncogene occurring in cis (on the same coy of gene). Panel A: Schematic of Imatinib resistance in CML (adapted from Gorre et al., Science. 2001 Aug. 3; 293(5531):876-80). Panel B: Schematic of EGFRi resistance in lung cancer (adapted from Koayashi et al., N Eng J Med 2005; 352: 786-792). Panel C: Schematic of BRAFi resistance in melanoma (adapted from Pouilikakos et al., Nature. 2011 Nov. 23; 480 (7377): 387-90). Panel D: Schematic of drug-resistance mutation and oncogenic mutation occurring in cis.

FIG. 21 shows a non-limiting exemplary schematic of dimer-interface mutations and effects thereof. Dimer-interface can confer resistance to mutant IDH inhibitors.

FIG. 22 shows non-limiting data and schematics of IDH2 dimer-interface mutations. IDH2 dimer-interface mutations in cis can biochemically confer drug resistance.

FIG. 23 shows non-limiting schematics and data of dimer-interface mutations in cis.

FIG. 24 shows non-limiting illustrations and graphs of IDH2 and IDH2 RQ/IM data.

FIG. 25 shows a non-limiting illustration of dimer-interface mutations in cis and graphs of isocitrate oxidation with NAD+ and 2HG production with NADH.

FIG. 26 shows exemplary crystal structure illustrations of IDH2 mutants and an image of IDH2 crystals.

FIG. 27 shows exemplary crystal structures of IDH2 R140Q and IDH2 R140Q/I319M.

FIG. 28 shows exemplary crystal structure of IDH2 R140Q and IDH2 R140Q/I319M.

FIG. 29 shows exemplary illustrations of interactions between R288^{A} and R353^{B} interactions in IDH2 R140Q and IDH2R140Q/I319M.

FIG. 30 shows exemplary cryo-EM data of active-site mutant IDH2 R140Q.

FIG. 31A-D shows exemplary cryo-EM data of the cis-mutant IDH2 R140Q/I319M.

FIG. 32 shows exemplary aligned cryo-EM maps (apo enzyme) for IDH2 RQ and IDH2 RQ/IM. The cis-mutant IDH2 adopts a closed confirmation compared to the active-site mutant.

FIG. 33 shows an exemplary illustration of an overlay of cryo-EM structures (apo enzyme) for IDH2 RQ and IDH2 RQ/IM.

FIG. 34 shows a non-limiting schematic, fluorescence data, and a western blot comparing allelic configurations.

FIG. 35 shows non-limiting exemplary data on the effects of allelic configurations on leukemia cell growth.

FIG. 36 shows non-limiting, exemplary data comparing effects of allelic configurations.

FIGS. 37A and B shows non-limiting, exemplary data for allelic configurations. Panel A shows exemplary results of gene set enrichment analysis (GSEA). Panel B shows an image of the results of a gel electrophoresis experiment.

FIG. 38 shows a schematic and representative data comparing IDH2 dimer-interface mutations.

FIG. 39 shows data comparing the effects of the IDH2 dimer-interface mutation in cis.

FIG. 40 shows exemplary heat maps for IDH2 pre, trans, and cis allelic configurations and a schematic of a metabolic pathway.

FIG. 41 shows, without wishing to be bound by theory, an exemplary graph comparing the NADH consumption in a control (vehicle) and in a small molecule activator of mutant IDH2.

FIG. 42 shows non-limiting, exemplary compound screening results and structures thereof.

FIGS. 43A-B and C shows non-limiting, exemplary data of a small molecule that can activate mutant IDH2. Panel A shows a graph of NADPH-dependent 2HG production. Panel B shows graphs of NADH-dependent 2HG production. Panel C shows a graph of high throughput screen results.

FIG. 44 shows a non-limiting, exemplary illustration of binding sites in mutant IDH2.

FIG. 45 shows non-limiting, exemplary illustrations of the structural basis for IDH2 activation.

FIG. 46 shows non-limiting, exemplary results experiments in Ba/F3 mouse leukemia cells transduced with IDH2 WT or R140Q.

FIG. 47 shows graphs of toxicity studies in MLL-AF9 and TF1 cells.

FIG. 48 shows non-limiting, exemplary results of SAR-guided lead optimization.

FIG. 49 shows non-limiting, exemplary data of the effect compounds on purified IDH2 R140Q enzyme.

FIG. 50 shows non-limiting, exemplary data of the effects of EDBN on IDH2 R172X mutations.

FIG. 51 shows a schematic of therapeutic strategies (adapted from Intlekofer, Shih et al, Nature 2018 and Harding . . . Intlekofer, Cancer Discovery 2018).

FIG. 52 shows a schematic of hyperactivation instead of inhibition for mutant IDH enzymes.

### DETAILED DESCRIPTION OF THE INVENTION

Detailed descriptions of one or more preferred embodiments are provided herein. It is to be understood, however, that the present invention can be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” can refer to approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn towards compositions and methods for preventing, treating, or ameliorating a symptom of cancer in a subject. For example, embodiments as described herein comprise inducing activity of an enzyme in the cancer cells, thereby preventing, treating, or ameliorating a symptom of cancer in the subject. For example, the cancer to be treated by the methods described herein can comprise acute myeloid leukemia, glioma, cholangiocarcinoma, chondrosarcoma, T cell lymphoma, a cancer comprising mutIDH1, a cancer comprising mutIDH2, or a combination thereof. For example, the cancer comprising mutIDH1 can comprise R132, SNP rs11554137, R100, G97D, and Y139D (see, e.g., Ward et al., Identification of additional IDH mutations associated with oncometabolite R(−)-2-hydroxyglutarate production. Oncogene. 2012 May 10; 31(19):2491-8). For example, the cancer comprising mutIDH2 can comprise R140 and R172 (see, e.g., Ward et al.).

The term “cancer” can refer to diseases in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue, such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord. Accordingly, aspects of the invention are drawn towards compositions and methods for preventing, treating, or ameliorating a symptom of a carcinoma, a sarcoma, a leukemia, a lymphoma, a myeloma, or a central nervous system cancer.

In embodiments, the cancer can be a solid tumor or a liquid cancer. A “solid tumor”, which can also be referred to as a “solid organ cancer”, can refer to an abnormal mass of tissue that usually does not contain cysts or liquid. A “non-solid tumor”, which can be referred to as a “liquid cancer,” can refer to neoplasia of the hemopoietic system, such as lymphoma, myeloma, and leukemia, or neoplasia without solid formation generally and with spread substantially.

Non-limiting examples of solid tumors comprise brain cancer, lung cancer (e.g., non-small cell lung cancer), liver cancer, hepatocellular carcinoma (HCC), esophageal cancer, cholangiocarcinoma, gallbladder carcinoma, stomach cancer, abdominal cancer, gastrointestinal cancer, gastric cancer, pancreatic cancer, renal cell carcinoma, renal cancer, bone cancer, breast cancer, ovarian cancer, uterine cancer, cervical cancer, endometrial cancer, colorectal cancer, colon cancer, rectal cancer, bladder cancer, superficial bladder cancer, prostate cancer, adrenal tumors, squamous cell carcinoma, neuroma, malignant neuroma, myoepithelial carcinoma, synovial sarcoma, rhabdomyosarcoma, gastrointestinal interstitial cell tumor, skin cancer, basal cell carcinoma, malignant melanoma, thyroid cancer, nasopharyngeal carcinoma, hemangioma, epidermoid carcinoma, head and neck cancer, glioma, or Kaposi's sarcoma. For example, the cancer comprises lung cancer (e.g., non-small cell lung cancer), prostate tumor, ovarian tumor, or pancreatic tumor.

Non-limiting examples of non-solid tumors or liquid cancers comprise leukemia, acute leukemia, chronic leukemia, chronic myelocytic leukemia, acute myelogenous leukemia, chronic myelogenous leukemia, chronic lymphocytic leukemia, acute lymphoblastic leukemia, T-cell leukemia, hairy cell leukemia, polycythemia, myelodysplastic syndrome, multiple myeloma, lymphadenoma, Hodgkin's lymphoma, and Non-Hodgkin's lymphoma.

In embodiments, aspects of the invention are drawn towards compositions and methods to treat, prevent, or ameliorate the symptoms of a subject afflicted with a lung tumor, including but not limited to abnormally proliferative or aberrantly proliferative lung cells and/or malignant lung tumor cells. The term “lung tumor” can refer to any lung tumors, including but not limited to primary lung tumors and/or metastatic lung tumors. For example, metastatic lung tumors can be those that have formed in a way that tumors at other positions are metastasized to the lung through various metastasis modes. The lung tumors can be benign (non-cancerous), preinvasive lesion (precancerous lesion), or malignant (carcinous) lung tumors, such as lung cancers. In embodiments, the lung cancer comprises non-small cell lung cancer.

Aspects of the invention are drawn to compositions and methods for treating a subject afflicted with cancer. The term “treat,” “treatment,” and/or “treating” can refer to the management and care of a subject for the purpose of combating a condition, disease, or disorder, in any manner in which one or more of the symptoms of a disease or disorder are ameliorated or otherwise beneficially altered. The term can include the full spectrum of treatments for a given condition from which the patient is suffering, such as administration of an active agent for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications. The subject to be treated can be a mammal, such as a human being. Treatment can also encompass any pharmaceutical use of a composition described herein, such as use for treating a disease as provided herein.

The term “treatment of cancer” or “treating cancer” can refer to the prevention or alleviation or amelioration of any of the phenomena known in the art to be associated with the pathology commonly known as “cancer.” As described herein, the term “cancer” can refer to the spectrum of pathological symptoms associated with the initiation or progression, as well as metastasis, of malignant tumors. As described herein, the term “tumor” can refer to a new growth of tissue in which the multiplication of cells is uncontrolled and progressive. In embodiments, the cancer can be a malignant cancer, one in which the primary cancer has the properties of invasion or metastasis, or which shows a greater degree of anaplasia than do benign cancers. In embodiments, the cancer can be a solid tumor or a non-solid tumor. In embodiments, the cancer can be a drug-resistant cancer, such as a checkpoint inhibitor resistant cancer. Thus, “treatment of cancer” or “treating cancer” can refer to an activity that prevents, alleviates or ameliorates any of the primary phenomena (initiation, progression, metastasis) or secondary symptoms associated with the disease.

Treating cancer can be indicated by, for example, inhibiting or delaying invasiveness of a cancer. “Cancer invasion” can refer to the movement caused by cancer cells in vivo, into or through biological tissue or the like. For example, movements caused by cancer cells into or through barriers formed by special cell-based proteins, such as collagen and Matrigel, and other substances.

The term “preventing cancer” can refer to prevention of cancer occurrence in a subject. In embodiments, the preventative treatment reduces the recurrence of the cancer. In embodiments, preventative treatment decreases the risk of a patient from developing a cancer or inhibits progression of a pre-cancerous state (e.g., a colon polyp) to actual malignancy.

The terms “individual”, “patient” and “subject” can be used interchangeably. They can refer to a mammal (e.g., a human) which is the object of treatment, or observation. Typical subjects to which compositions and methods described herein can be administered will be mammals, for example primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like.

Mutant Isocitrate Dehydrogenase (mutIDH), Inhibitors of mutIDH, and Resistance to the Inhibitors

Isocitrate dehydrogenase (IDH) is an enzyme that catalyzes oxidative decarboxylation of isocitrate, producing alpha-ketoglutarate and carbon dioxide. IDH can exist as three isoforms: IDH1 (Gene ID: 3417; Protein Accession No. NP_005887), IDH2 (Gene ID: 3418; Protein Accession No. NP_002159), and IDH3 (Gene ID: 3419; Protein Accession No. P50213). IDH1 encodes a cytosolic protein while, IDH2 encodes a mitochondrial protein. Functional IDH enzymes can be homodimers. Mutations in endogenous isocitrate dehydrogenase enzymes can lead to the production of the oncometabolite 2-hydroxyglutarate (2HG). As used herein, the term “oncometabolite” can refer to a pathognomonic hallmark in cancer. Such mutant IDH molecules can be referred to as mutIDH. In some embodiments, the oncometabolite can initiate cancer or cause the cell to become a cancer cell. Therefore, mutant mutIDH molecules can have an oncogenic activity. A mutIDH can have more than one activity (e.g., a toxic activity). In some embodiments, the mutations in IDH to produce mutIDH can be called active-site mutations.

An IDH1 or IDH2 heterodimer can have: i) two wild-type (e.g., non-mutated) subunits, ii) a wild-type and a mutant subunit, or iii) two mutant subunits. Both the (ii) and (iii) heterodimers are mutIDH enzymes that can produce 2HG and/or initiate cancer or cause cells to become cancer cells.

In embodiments, the mutIDH can be mutIDH1 or mut IDH2. As used herein, the term “mutIDH1” can refer to mutant IDH1. In embodiments, the mutIDH1 can comprise an amino acid substitution. In embodiments, the amino acid substitution can lead to an oncogenic mutation. As used herein “oncogenic mutation” can refer to a mutation which can lead to carcinogenesis. Non-limiting examples of oncogenic mutations in IDH1 can occur at R132, V71 or R100, G97D, Y139D, and SNP rs11554137. Other oncogenic mutations in IDH1 can occur.

As used herein, the term “mutIDH2” can refer to mutant IDH2. In embodiments, the mutIDH2 can comprise an amino acid substitution. In embodiments, the amino acid substitution can lead to a mutation. In embodiments, the mutation can be an oncogenic mutation. Non-limiting examples of oncogenic mutations in IDH2 can occur at R140 or R172. Other oncogenic mutations in IDH2 can occur.

In some embodiments, IDH1 and IDH2 can be paralogous to one another. In some embodiments, substitution of certain amino acid positions in IDH1 can produce cancer. The substituted amino acids positions can have homologous amino acid positions in IDH2. In some embodiments, substitution of certain amino acid positions in IDH2 can produce cancer. The substituted amin acid positions can have homologous amino acid position in IDH1.

Allosteric inhibitors, called mutant IDH inhibitors (mutIDH inhibitors), can be used to treat cancers that have a mutIDH. In some embodiments, the mutIDH inhibitors can decrease an amount of 2HG in the cancer cells. In some embodiments, the mutIDH inhibitors can decrease 2HG by inhibiting the mutIDH from producing 2HG. The allosteric inhibitors may not bind to the active site of the mutIDH. In some embodiments, a mutIDH inhibitor can be Ivosidenib (inhibitor for mutIDH1), Ensaidenib (inhibitor for mutIDH2) or another molecule.

Additional non-limiting, exemplary mutIDH1 inhibitors can be BAY1436032, FT-2102, IDH305, AGI-5198, ML309 (AGI-5027), GSK 321, DC_H31, and AGI-5198. Other non-limiting mutIDH1 inhibitors can include indane analogs, IDH305, AG-881, BAY1436032, FT-2102, VVS, GSK-321, DS-1001b, 3-aryl-4-indolyl-maleimides, SYC-435, Compound 13, HMS-101, DC_H31, WM-17, CRUK-MI, ZX-06, L806-0255, V015-1671, AQ-714/41674992 and the like.

Additional non-limiting, exemplary mutIDH2 inhibitors can be AGI-6780 and the like.

In embodiments, the mutIDH inhibitor is an allosteric inhibitor. As used herein, the term “allosteric inhibitor” can refer to a molecule that binds to an allosteric site. As used herein, the term “allosteric site” can refer to a site other than an active site. In embodiments, the mutIDH inhibitor does not bind to an active site of the mutIDH.

The term “inhibitor” can refer to a substance having an inhibitory activity against the function of a target molecule such as a compound, an antibody, an anti-sense oligonucleotide (“Antisense Drug Technology: Principles, Strategies, and Applications (Second Edition)”, CRC Press, 2007), an RNAi oligonucleotide (“RNA Methodologies (Third Edition)”, Elsevier, 2005, Chapter 24), a peptide nucleic acid (Kaihatsu et al., Chemistry & Biology, 2004, 11 (6), p. 749-758) and a peptidic antagonist (Ladner et al., Drug Discovery Today, 2004, 9, p. 525-529). Accordingly, inhibitors can encompass numerous classes of chemical molecules, e.g., small organic or inorganic molecules, polysaccharides, biological macromolecules, e.g., peptides, proteins, peptide analogs and derivatives, peptidomimetics, antibodies, antibody fragments, nucleic acids, nucleic acid analogs and derivatives, an extract made from biological materials such as bacteria, plants, fungi, or animal cells or tissues, naturally occurring or synthetic compositions.

Cellular resistance to these mutant IDH inhibitors can develop. In some embodiments, resistance to mutIDH inhibitors can be caused by secondary mutations in the mutIDH. In some embodiments, the secondary mutations can occur in the dimer-interface of the IDH enzymes (the region where the two subunits come together to form the functional mutIDH enzyme). Such mutations in the dimer-interface can disrupt mutIDH inhibitor binding to mutIDH. The mutations can restore production of 2HG (Intlekofer, Shih et al, Nature 2018) that can be responsible for the resistance. In some embodiments, the secondary mutations that make mutIDH enzymes resistant to IDH inhibitors can be called dimer-interface mutations. In some embodiments, these secondary mutations can be amino acid substitutions at S280 for mutIDH1. In some embodiments, amino acid substitutions at S280 (e.g., S280F) can make mutIDH1 resistant to ivosidenib or other mutIDH inhibitors. In some embodiments, these secondary mutations can be amino acid substitutions at Q316 and/or I319 for mutIDH2. In some embodiments, amino acid substitutions at Q316 (e.g., Q316E) and/or I319 (e.g., I319M) can make mutIDH2 resistant to ensaidenib or other mutIDH inhibitors.

In some embodiments, other secondary mutations that can make mutIDH1 resistant to mutIDH inhibitors (e.g., ivosidenib) can be amino acid substitutions at R119 (e.g, R119P), G131 (e.g., G131A), D 279 (e.g., D279N), G289 (e.g., G289D), T313 (e.g., T313I), H315 (e.g., H315D), and others.

In some embodiments, other secondary mutations that can make mutIDH2 resistant to mutIDH inhibitors (e.g., ensaidenib) can be amino acid substitutions at N136 (e.g., N136S), D279 (e.g., D279N), E343 (e.g., E343V), E345 (e.g., E345G), A347 (e.g., A347T), H348 (e.g., H348Q), T352 (e.g., T352A), R353 (e.g., R353H), and others.

Other mutations in patients developing resistance to mutIDH inhibitors can be found within the genes encoding IDH1 and IDH2, as well as elsewhere in the genome.

Without wishing to be bound by theory, the two subunits forming a mutIDH that can produce 2HG (i.e., catalytically active form) can be said to be in a more “closed” conformation. MutIDH inhibitors can stabilize the two subunits forming a mutIDH dimer in a more “open” conformation, which is not catalytically active to produce 2HG. Mutations in mutIDH that make the enzyme resistant to mutIDH inhibitors can affect the ability of mutIDH inhibitors to bind to the mutIDH enzyme. The resistance mutations allow the enzyme to form the catalytically active, closed conformation.

As used herein, “drug resistance” can refer to a condition in which the disease does not respond to treatment with one or more drugs. Accordingly, aspects of the invention are drawn to compositions and methods for treating, preventing, or ameliorating the symptoms of a subject afflicted with a drug-resistant cancer. For example, the subjects can be afflicted with cancers that have acquired resistance to mutant inhibitors

For mutations in IDH2, an observation was the allelic configuration wherein dimer-interface mutations (responsible for resistance to mutIDH inhibitors) in mitochondrial IDH2 occurred in trans (on the other allele) relative to the active-site mutation (oncogenic mutation) responsible for 2HG production. In cis arrangements of these mutations did not appear to occur. This contrasts with the universal in cis (on the same allele) configuration of mutations in oncogenes across cancers (Gorelick et al, Nature 2020). As used herein, the terms “in cis” and “cis” can be used interchangeably. As used herein, “in cis” can refer to mutations occurring on the same allele. As used herein, the terms “in trans” and “trans” can be used interchangeably. As used herein, “in trans” can refer to mutations occurring on different alleles.

We found that in cis arrangements of a mutIDH2 oncogenic mutation and a mutIDH inhibitor resistance mutation could be made and tested in cultured cells. This arrangement: i) produced very high levels of 2HG; ii) allowed the mutIDH2 to use NADH in addition to its normal NADPH cofactor; iii) caused mitochondrial and metabolic dysfunction; and iv) inhibited cell proliferation. Because at least some of the effects produced by the in cis arrangement was toxic to cells, these data indicated that “hyperactivating” a mutIDH2 could be a therapeutic strategy in cancers having these oncogenic mutations (the active-site mutations), and in cancers having these oncogenic mutations as well as mutations making the cancer cells resistant to mutant IDH inhibitors (the dimer-interface mutations).

### Methods for Treating Cancers

Described herein are compositions and methods for treating cancer and/or preventing cancer in a subject. In some embodiments, the compositions and methods are designed to increase the amount and/or activity of an enzyme in a cancer cell. In some embodiments, the enzyme can be a mutant enzyme. In some embodiments, the mutant enzyme can have toxic activity to a cell. In some embodiments the mutant enzyme can be a mutant isocitrate dehydrogenase (mutIDH). In some embodiments, the mutIDH can produce 2HG. In some embodiments, the methods rely on inducing or hyperactivating (i.e., not inhibiting) the mutIDH or one or more activities of a mutIDH. In some embodiments, the methods do not rely on inhibition of an oncogenic activity of a mutIDH.

“Oncogenic activity” of a mutIDH can refer to an activity that can cause a non-cancerous cell to become a cancerous cell. In some embodiments, an oncogenic activity of mutIDH can be, at least in part, due to production of 2HG by a mutIDH. Without wishing to be held to any theory, a specific amount of 2HG in a cell can cause cancer. Without wishing to be held to any theory, a different amount of 2HG in a cell can be toxic to the cell. Without wishing to be held to any theory, an amount of 2HG that can cause cancer in a cell can be less than an amount of 2HG that is toxic to a cell. In some embodiments, higher amounts of 2HG can be toxic.

In some embodiments, mutIDH can be found, at least at low frequency, in many different types of cancer. Herein, the disclosed compositions and methods for treating cancer can be applied to any cancer having a mutIDH and containing 2HG. In some embodiments, the cancer can be acute myeloid leukemia, glioma, cholangiocarcinoma, chondrosarcoma or T cell lymphoma.

As used herein, the term “inducing” can refer to increasing and/or enhancing an activity of an enzyme. As used herein, the term “hyperactivating” can refer to increasing and/or enhancing an activity of an enzyme to a degree not normally reached in a cell. In some embodiments, an activity of an enzyme that is hyperactivated can be detrimental to a cell or its physiology. For example, the methods described herein can comprise hyperactivating a mutIDH in a cancer cell.

In some embodiments, the mutIDH that is hyperactivated is already in a cancer cell (e.g., an endogenous, non-oncogenic IDH becomes a mutIDH by acquiring a mutation and an oncogenic activity). In some embodiments, a gene encoding a hyperactivated mutIDH can be introduced into the cancer cells. In some embodiments, the hyperactivated mutIDH gene can have an active-site mutation such that the mutIDH enzyme can produce 2HG, and a dimer-interface mutation such that the mutIDH enzyme is resistant to a mutIDH inhibitor. In some embodiments, these two mutations are in encoded by the same allele. In these methods, the hyperactivated mutIDH can be detrimental to the cancer cells. In some embodiments, this approach—hyperactivating an oncogenic protein—is counterintuitive to previous approaches to treat cancer that have sought to inhibit, rather than induce or hyperactivate oncogenic enzymes in cancer cells.

In embodiments, the enzyme that is hyperactivated using the disclosed methods can be an endogenous enzyme of the cancer cells. As used herein, the term “endogenous” can refer to a material originating in and/or produced by an organism, tissue, and/or cell. In embodiments, the endogenous enzyme does not have oncogenic activity. In embodiments, the endogenous enzyme can be IDH1 or IDH2. One or more mutations in the endogenous enzyme can cause the endogenous enzyme to have oncogenic activity. In embodiments, the mutant enzyme can be a mutant isocitrate dehydrogenase (mutIDH). The enzyme can be mutIDH1 or mutIDH2. In embodiments, the mutIDH can catalyze the reaction of α-ketoglutarate to 2-hydroxyglutarate (2HG). Production of 2HG can initiate/cause the cancer. Such a mutIDH can be said to have oncogenic activity.

In embodiments, inducing activity or hyperactivity of a mutated enzyme can be detrimental to a cell. In some embodiments, the cell can be a cancer cell. In some embodiments, inducing activity/hyperactivity of the mutated enzyme can be used as a treatment for the cancer. In some embodiments, inducing activity/hyperactivity of the mutIDH can increase the amount of 2HG in cancer cells and/or increase an activity of the mutIDH that is detrimental to the cancer cells. In some embodiments, inducing activity/hyperactivity of the mutIDH can deplete α-ketoglutarate (αKG or AKG), nicotinamide adenine dinucleotide (NADH) and/or nicotinamide adenine dinucleotide phosphate (NADPH) from the mitochondria of the cells.

While not wishing to be bound by a particular theory, detriment to a cell by hyperactivating a mutIDH can be due or partially due to increased amounts of 2HG. Detriment to a cell by hyperactivating a mutIDH can be due or partially due to another activity of the mutIDH.

Aspects of the invention are drawn towards inducing activity of a mutIDH in the cells of the cancer and comprises contacting the cells of the cancer with a molecule that can increase an amount of 2HG in the cells of the cancer and/or can deplete αKG, NADPH, or NADH. In embodiments, the molecule can facilitate the mutIDH to change its cofactor preference. As used herein, the phrase “changing a cofactor preference” can refer to changing a NADPH dependent reaction to an NADH dependent reaction. For example, the molecule allows use of NAD/NADH in addition to the preferred cofactors NADP/NADPH by the mutIDH. As used herein, the term “cofactor” can refer to a substance that is necessary or beneficial to the activity of an enzyme.

### Compositions for Treating Cancers

In some embodiments, disclosed are compositions that can hyperactivate an oncogenic activity of an enzyme in cancer cells. The hyperactivation can be toxic to the cells. In some embodiments, the compositions can contain a small molecule(s) that can hyperactivate the activity of the enzyme. In some embodiments, hyperactivation of an oncogenic activity of an enzyme in cancer cells can use genetic modification of the cancer cells.

Aspects of the invention are drawn towards a method of treating a cancer in a patient, where the cancer cells contain an isocitrate dehydrogenase (IDH) that can make 2-hydroxyglutarate (2HG) from α-ketoglutarate (αKG). Such an IDH is a mutIDH. The method can include administering to the patient a composition containing a molecule that can affect activity of the IDH in the cells of the cancer. In embodiments, the compositions (e.g., containing a small molecule) are specific for cells containing the mutIDH. In embodiments, the mutIDH comprises mutIDH or mutIDH2. In embodiments, the composition can hyperactivate the mutIDH in the cells of the cancer. For example, the composition can cause mutIDH to synthesize an increased amount (e.g., hyperactivated amount) of 2-hydroxyglutarate (2HG) in the cells of the cancer. For example, the composition can induce excessive consumption of NADPH, NADH and α-ketoglutarate in the cells of the cancer. In embodiments, the composition can change a cofactor preference for the mutIDH. In embodiments, the composition can impair growth and/or proliferation of the cells of the cancer, cause mitochondrial dysfunction in the cells of the cancer and/or cause metabolic dysfunction in the cells of the cancer. For example, the mitochondrial disfunction can comprise collapse of TCA-cycle metabolite pools. Decreased nucleotide intermediates and numerous amino acids can also exist. For example, the metabolic dysfunction can comprise decrease in oxygen consumption and lower reactive oxygen species (ROS). Long-term, increased mitochondrial reactive oxygen species can exist.

Small molecules that can hyperactivate a mutIDH can bind to mutIDH. In some embodiments, the small molecules can bind to or interact with a site in a mutIDH that is different from a site where a mutIDH inhibitor can bind. In some embodiments, bind/interact can refer to an intermolecular force and/or an intramolecular force. As used herein, “intermolecular force” can refer to forces between molecules. For example, the intermolecular force can comprise dispersion forces, hydrogen bonding, dipole-dipole forces. As used herein, “intramolecular force” can refer to forces within molecules. For example, the intramolecular force can comprise an ionic bond, a covalent bond, and a metallic bond.

In embodiments, the inhibitor can be a small molecule inhibitor. The term “small molecule” can refer to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (e.g., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

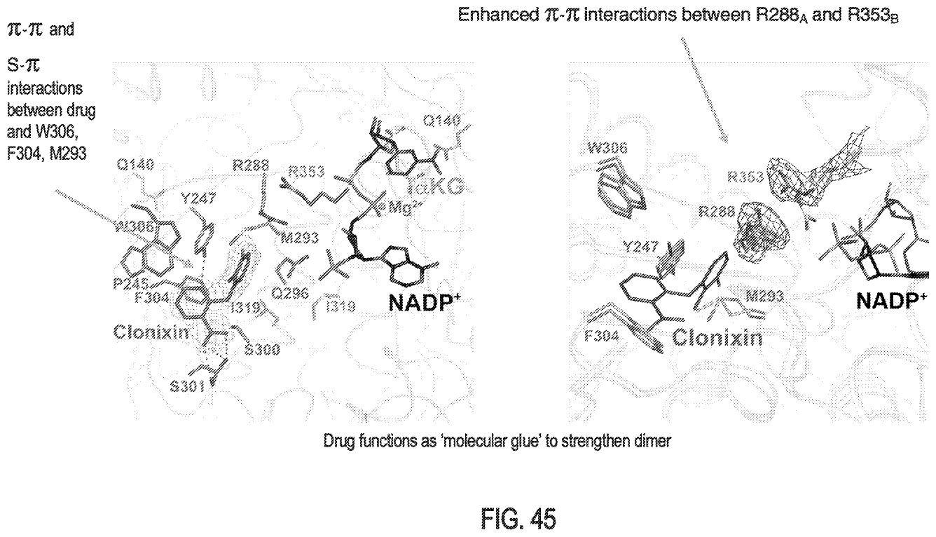

In some embodiments, the small molecules that hyperactivate mutIDH can bind to a small hydrophobic pocket of mutIDH. For mutIDH2, in some embodiments, the small molecules can interact with amino acid residues S200, K243, W244, P245, Y247, K282, W284, R288, M293, Q296, S301, G303, F304, and/or W306 (e.g., FIG. 45). In some embodiments, analogous or homologous amino acids from mutIDH1 can interact with the small molecules. Table 1, below, shows some of these homologous amino acids in mutIDH1 and mutIDH2 related to amino acid positions contacted by example small molecules that hyperactivate the mutIDH enzymes.

TABLE 1
Some homologous
amino acid positions in
mutIDH2 and mut IDH1

| mutIDH2 | mutIDH1 |
|---|---|
| K242 | K203 |
| K243 | G204 |
| W244 | W205 |
| P245 | P206 |
| Y247 | Y208 |
| K282 | K243 |
| W284 | W245 |
| R288 | R249 |
| M293 | M254 |
| Q296 | Q257 |
| S300 | S261 |
| S301 | E262 |
| G303 | G264 |
| F304 | F265 |
| W306 | W267 |

The interactions between the small molecules and the mutIDH can be π-π interactions, polar interactions, and the like. In embodiments, the small molecules binding to mutIDH2 can reposition R288 of one subunit and R353 of the second subunit to enhance π-π binding across the dimer interface of the mutIDH. In some embodiments, the two mutIDH subunits move closer to one another due to the small molecule binding. Binding of the small molecules to the IDH can strengthen intermolecular interactions within the dimer interface of the mutIDH. In some embodiments, the small molecule binding to mutIDH2 can cause the two enzymatic subunits to move closer to one another by 0.1, 0.2, 0.4, 0.5, 0.6, 0.7, 0.8 or 0.9 Å. In some embodiments, the subunits can move closer to one another by 0.1-0.9 Å, 0.2-0.8 Å or 0.3-0.6 Å. In some embodiments, the subunits moving closer to one another can be measured by the distance between two I319-Cα atoms. In some embodiments, these measurements can be obtained from of crystals of the mutIDH and small molecule.

In some embodiments, the small molecule binding can shift a Q140 residue in IDH2 (or homologous residues in IDH1) toward a closed conformation by about 2.8, 3.0, 3.2, 3.3, 3.4, 3.5, 3.6, 3.7, 3.8, 3.9, 4.0, 4.1, 4.3 or 4.5 Å. In some embodiments the residue in IDH2 or IDH1 can shift toward a closed conformation by about 2.8-4.5 Å, 3.0-4.3 Å, 3.2-4.1 Å, 3.3-4.0 Å, 3.4-3.9 Å or 3.5-3.8 Å.

This configuration of the mutIDH can be referred to as a “closed” conformation. In embodiments, the closed conformation is catalytically active. In embodiments, contact of the mutIDH by the small molecule can hyperactivate the mutIDH.

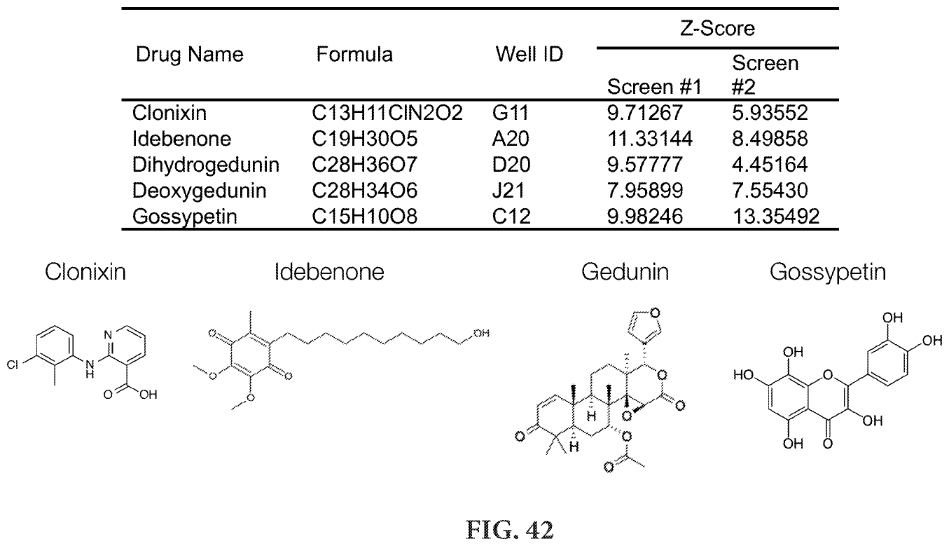

In some embodiments, the small molecules that hyperactivate mutIDH can be clonixin, idebenone, dihydrogedunin, deoxygedunin, gossypetin, derivatives thereof, related compounds and/or combinations thereof (FIG. 42).

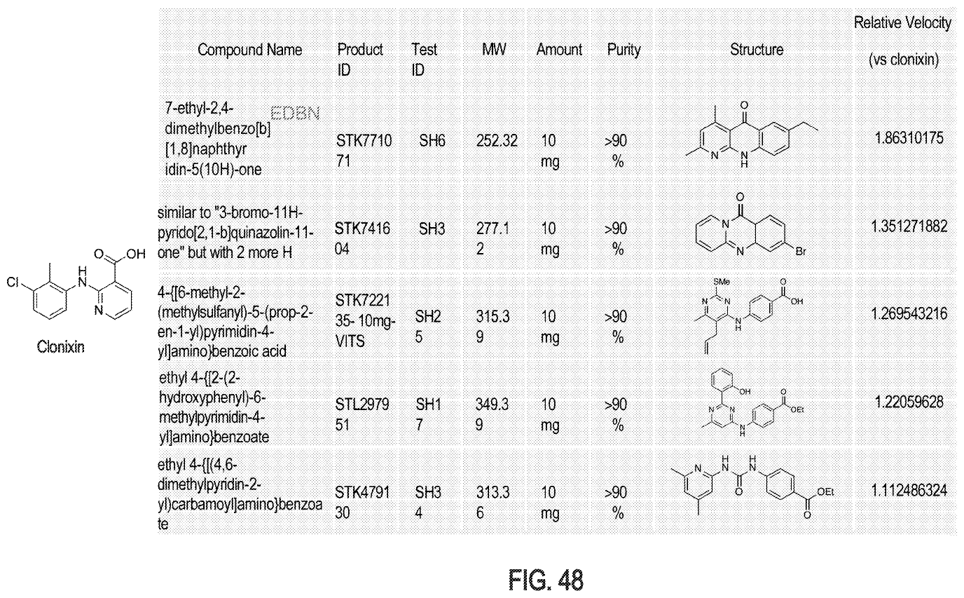

In some embodiments, the small molecules can be molecules shown in FIG. 48, derivates thereof, related compounds and/or combinations thereof. In some embodiments, the small molecules can be 7-ethyl-2,4-dimethylbenzo[b][1,8]naphthyridin-5(10H)-one (EDBN); 4-{[6-methyl-2-(methylsulfanyl)-5-(prop-2-en-1-yl)pyrimidin-4-yl]amino}benzoic acid; ethyl 4-{[2-(2-hydroxyphenyl)-6-methylpyrimidin-4-yl]amino}benzoate; ethyl 4-{[(4,6-dimethylpyridin-2-yl)carbamoyl]amino}benzoate; derivatives thereof, related compounds and/or combinations thereof.

In some embodiments, the small molecule can be clonixin ([2-(3-chloro-o-toluidino)nicotinic acid] or 2-(2′-methyl-3′-chloro)-anilino-nicotinic acid), derivatives thereof and related compounds that can induce mutIDH2 in cells of the cancer that have an R140 mutation in the IDH2.

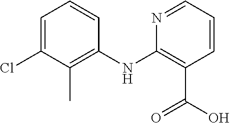 [SMILES: Cc1c(Cl)cccc1Nc1ncccc1C(=O)O]

### Clonixin

In some embodiments, the small molecule can be EDBN (7-ethyl-2,4-dimethylbenzo[b][1,8]naphthyridin-5(10H)-one), derivatives thereof and related compounds that can induce mutIDH2 in cells of the cancer that have an R172 mutation in the IDH2.

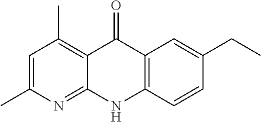 [SMILES: CCc1ccc2[nH]c3nc(C)cc(C)c3c(=O)c2c1]

### 7-ethyl-2,4-dimethylbenzo[b][1,8]naphthyridin-5(10H)-one (EDBN)

In some embodiments, the small molecules can be clonixin, derivatives thereof, or related compounds. Derivatives and compounds related to clonixin can include the molecules illustrated at this website: pubchem.ncbi.nlm.nih.gov/compound/Clonixin #section=Related-Compounds-with-Annotation&fullscreen=true.

In embodiments, the compound that can activate/hyperactivate mutIDH can be clonixin, EDBN, and derivatives thereof. In some embodiments, the clonixin, EDBN, and derivatives thereof can hyperactivate a mutIDH (e.g., mutIDH1, mutIDH2) in cells of a cancer. In some embodiments, the cells of the cancer may not have a secondary mutation in the mutIDH. In some embodiments, the cells of the cancer may have a secondary mutation in the mutIDH (e.g., a mutation that makes the mutIDH resistant to a mutIDH inhibitor).

Aspects of the invention are drawn towards preventing and/or treating cancer in a subject by administering a composition that can induce forced expression of a dimer-interface mutation with an active-site mutation. In some embodiments, such forced expression can be accomplished by genetic modification of the cancer cells. In some embodiments, a nucleic acid encoding a hyperactivated mutIDH can be introduced into the cancer cells. In some embodiments, a gene encoding a hyperactive mutIDH in a cancer call can be activated by genetic modification. The hyperactivated mutIDH can produce 2HG.

In some embodiments, a gene encoding a hyperactivated mutIDH can have an oncogenic mutation (an active-site mutation) and a mutIDH inhibitor resistance mutation (a dimer-interface mutation). These mutations can be on the same allele (i.e., in cis on the same gene).

In some embodiments, the gene encoding the hyperactivated mutIDH can be a mutIDH2 encoding an amino acid substitution at R140 or R172, and encoding an amino acid substitution at Q316 or I319. The two mutations can be in cis on the gene.

In some embodiments, the genes can be introduced into the cancer cells using retroviral or lentiviral vectors, or by CRISPR-based gene editing.

### Administration of the Compositions

As described herein, aspects of the invention are drawn to methods of administering a therapy (e.g., composition that can induce forced expression of a dimer-interface mutation in cis with an active-site mutation) to a subject afflicted with a cancer. The term “administer” can refer to can refer to introducing a composition or pharmaceutical composition as described herein into a subject. One route of administration of the composition is intravenous administration. However, any route of administration, such as oral, topical, subcutaneous, peritoneal, intra-arterial, inhalation, vaginal, rectal, nasal, introduction into the cerebrospinal fluid, or instillation into body compartments can be used.

The composition or pharmaceutical composition as described herein can be administered to the subject using any means that can result in the desired effect. Thus, the composition can be incorporated into a variety of pharmaceutical compositions for therapeutic administration. For example, the composition can be formulated into pharmaceutical compositions by combination with appropriate, pharmaceutically acceptable carriers, excipients, diluents, and/or adjuvants, and can be formulated into preparations in solid, semi-solid, liquid or gaseous forms, such as tablets, pills, capsules, powders, sustained release formulations or elixirs, granules, ointments, solutions, suspensions, suppositories, injections, inhalants and aerosols.

The composition or pharmaceutical composition can be administered alone, but can be administered with other compounds, excipients, fillers, binders, carriers or other vehicles selected based upon the chosen route of administration and standard pharmaceutical practice. Administration can be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; other proteins and peptides; synthetic polymers; microspheres; nanoparticles; and the like.

The composition or pharmaceutical composition can also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. For example, the pharmaceutically acceptable carriers, excipients, binders, and fillers that can be used include those which render the compounds of the invention amenable to intravitreal delivery, intraocular delivery, ocular delivery, subretinal delivery, intrathecal delivery, intravenous delivery, subcutaneous delivery, transcutaneous delivery, intracutaneous delivery, intracranial delivery, topical delivery and the like. Moreover, the packaging material can be biologically inert or lack bioactivity, such as plastic polymers, and silicone, and can be processed internally by the subject without affecting the effectiveness of the composition/formulation packaged and/or delivered therewith.

Different forms of the composition or pharmaceutical composition can be calibrated in order to adapt both to different subjects and to the different needs of a single subject. However, the compositions need not counter every cause in every individual. Rather, by countering the necessary causes, the compositions will restore the body and brain to their normal function. Then the body and brain themselves will correct the remaining deficiencies. No drug can correct every single aspect of cancer, but the compositions will maximize the possibility.

“Parenteral administration” can refer to administration via injection or infusion. Parenteral administration includes, but is not limited to, subcutaneous administration, intravenous administration, intramuscular administration.

For oral preparations, the composition or pharmaceutical composition can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Embodiments of the composition or pharmaceutical composition can be formulated into preparations for injection by dissolving, suspending, or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral administration, such as syrups, elixirs, and suspensions, can be provided wherein each dosage unit, for example, teaspoonful, tablespoonful, tablet or suppository, contains a predetermined amount of the composition containing one or more compositions. Similarly, unit dosage forms for injection or intravenous administration can comprise the composition or pharmaceutical composition in a composition as a solution in sterile water, normal saline or another pharmaceutically acceptable carrier.

Embodiments of the composition or pharmaceutical composition can be formulated in an injectable composition in accordance with the disclosure. For example, injectable compositions are prepared as liquid solutions or suspensions; solid forms suitable for solution in, or suspension in, liquid vehicles prior to injection can also be prepared. The preparation can also be emulsified or the active ingredient (triamino-pyridine derivative and/or the labeled triamino-pyridine derivative) encapsulated in liposome vehicles in accordance with the present disclosure.

In an embodiment, the composition or pharmaceutical composition can be formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Embodiments of the composition or pharmaceutical composition can be administered to a subject in one or more doses. Those of skill will readily appreciate that dose levels can vary as a function of the specific composition or pharmaceutical composition administered, the severity of the symptoms and the susceptibility of the subject to side effects. Dosages for a given compound are readily determinable by those of skill in the art by a variety of means.

In an embodiment, multiple doses of the composition or pharmaceutical composition are administered. The frequency of administration of the composition or pharmaceutical composition can vary depending on any of a variety of factors, e.g., severity of the symptoms, and the like. For example, in an embodiment, the composition or pharmaceutical composition can be administered once per month, twice per month, three times per month, every other week (qow), once per week (qw), twice per week (biw), three times per week (tiw), four times per week, five times per week, six times per week, every other day (qod), daily (ad), twice a day (qid), three times a day (tid), or four times a day. As discussed above, in an embodiment, the composition or pharmaceutical composition is administered 1 to 4 times a day over a 1 to 10-day time period.

The duration of administration of the composition or pharmaceutical composition analogue, e.g., the period of time over which the composition or pharmaceutical composition is administered, can vary, depending on any of a variety of factors, including patient response. For example, the composition or pharmaceutical composition in combination or separately, can be administered over a period of time of about one day to one week, about one day to two weeks. The amount of the combination therapy and pharmaceutical compositions of the disclosure that can be effective in treating the condition or disease can be determined by standard clinical techniques. In addition, in vitro or in vivo assays can optionally be employed to help identify optimal dosage ranges. The precise dose to be employed can also depend on the route of administration, and can be decided according to the judgment of the practitioner and each patient's circumstances.

As described herein, embodiments comprise administering to a subject a combination therapy comprising a composition that can induce forced expression of a dimer-interface mutation in cis with an active-site mutation in combination with a conventional therapy for a particular cancer. In embodiments, the two or more agents can be administered sequentially, such as one before the other, or concurrently or simultaneously, such as at about the same time. The term “simultaneous administration” can refer to the first agent and the second agent in the therapeutic combination therapy being administered either less than about 15 minutes, e.g., less than about 10, 5, or 1 minute. In embodiments, administration of the first agent and the second agent can overlap each other. When the first and second agents are administered simultaneously, the first and second treatments can be in the same composition (e.g., a composition comprising both the first and second therapeutic agents) or separately (e.g., the first therapeutic agent is contained in one composition and the second treatment is contained in another composition).

As described herein, the term “sequential administration” can indicate that the first agent and the second agent in combination therapy are administered greater than about 15 minutes apart, such as greater than about 20, 30, 40, 50, 60 minutes apart, or greater than 60 minutes apart. Either the first agent or the second agent can be administered first. The first and second agents can be included in separate compositions, which can be included in the same or different packages or kits.

Embodiments as described herein provide methods and compositions for the administration of the active agent(s) (e.g., a composition that can induce forced expression of a dimer-interface mutation in cis with an active-site mutation) to a subject using any available method and route suitable for drug delivery, including in vivo, in vitro and ex vivo methods, as well as systemic and localized routes of administration. Routes of administration include intranasal, intramuscular, intratracheal, subcutaneous, intra cerebroventricular, intradermal, topical application, intravenous, rectal, nasal, oral, and other enteral and parenteral routes of administration. Routes of administration can be combined, if desired, or adjusted depending upon the agent and/or the desired effect. An active agent can be administered in a single dose or in multiple doses.

Embodiments of the composition or pharmaceutical composition can be administered to a subject using available conventional methods and routes suitable for delivery of conventional drugs, including systemic or localized routes. Routes of administration can include, but are not limited to, enteral administration, parenteral administration, or inhalation.

Other compositions, compounds, methods, features, and advantages of the disclosure will be or become apparent to one having ordinary skill in the art upon examination of the following drawings, detailed description, and examples. It is intended that all such additional compositions, compounds, methods, features, and advantages be included within this description, and be within the scope of the disclosure.

Compositions and pharmaceutical compositions as described herein can be administered locally or systemically. “Local administration” can refer to administering a composition or drug into a limited or partial anatomy space. Examples of local administration include but are not limited to intratumoral, intra-lymph node, intra-pleural space, intraperitoneal cavity and the like. “Systemic administration” can refer to administration of an anti-cancer agent such that the anti-cancer agent becomes widely distributed in the body in significant amounts and has a biological effect, e.g., its desired effect, in the blood and/or reaches its desired site of action via the vascular system. For example, systemic routes of administration include administration by (1) introducing the agent directly into the vascular system or (2) oral, pulmonary, or intramuscular administration wherein the agent is adsorbed, enters the vascular system, and is carried to one or more desired site(s) of action via the blood.

Compositions described herein can be formulated into pharmaceutical compositions using techniques and procedures well known in the art (see, e.g., Ansel Introduction to Pharmaceutical Dosage Forms, Fourth Edition 1985, 126). A “pharmaceutical composition” can refer to a composition or pharmaceutical composition for administration to a subject, such as a mammal, especially a human and that can refer to the combination of one or more agents described herein with a pharmaceutically acceptable carrier or excipient, making the composition suitable for diagnostic, therapeutic, or preventive use in vitro, in vivo, or ex vivo.

A “pharmaceutical composition” can be sterile, and can be free of contaminants that can elicit an undesirable response within the subject (for example, the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intratracheal, intramuscular, subcutaneous, inhalational and the like.

Pharmaceutical compositions can further comprise an excipient, carrier, diluent, and/or adjuvant. A “pharmaceutically acceptable excipient,” “pharmaceutically acceptable diluent,” “pharmaceutically acceptable carrier,” or “pharmaceutically acceptable adjuvant” can refer to an excipient, diluent, carrier, and/or adjuvant that is useful in preparing a pharmaceutical composition that is safe, non-toxic and neither biologically nor otherwise undesirable, and can include an excipient, diluent, carrier, and adjuvant that is acceptable for veterinary use and/or human pharmaceutical use. See, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc. “A pharmaceutically acceptable excipient, diluent, carrier and/or adjuvant” as used in the specification and claims can include one and more such excipients, diluents, carriers, and/or adjuvants. For example, suitable excipient vehicles for the composition or pharmaceutical composition can be water, saline, dextrose, glycerol, ethanol, or the like, and combinations thereof.

In addition, if desired, the vehicle can contain minor amounts of pharmaceutically acceptable auxiliary substances, such as pH adjusting and buffering agents, tonicity adjusting agents, stabilizers, antioxidants, wetting agents and the like, are readily available to the public.

In embodiments, effective concentrations of the compositions described herein or pharmaceutically acceptable derivatives can be mixed with a suitable pharmaceutical carrier or vehicle. The compositions described herein can be derivatized as the corresponding salts, esters, enol ethers or esters, acids, bases, solvates, hydrates or prodrugs prior to formulation, as described herein. The concentrations of the active agents in the compositions can be effective for delivery of an amount, upon administration, that treats, prevents, or ameliorates one or more of the symptoms of a disease, disorder or condition, such as cancer.

Compositions can include those that comprise a sustained release or controlled release matrix. In addition, embodiments can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix. In another embodiment, the pharmaceutical composition (as well as combination compositions) can be delivered in a controlled release system. For example, the composition or pharmaceutical composition can be administered using intravenous infusion, an implantable osmotic pump, a transdermal patch, liposomes, or other modes of administration. In one embodiment, a pump may be used (Sefton (1987). CRC Crit. Ref. Biomed. Eng. 14:201; Buchwald et al. (1980). Surgery 88:507; Saudek et al. (1989). N. Engl. J. Med. 321:574). In another embodiment, polymeric materials are used. In yet another embodiment a controlled release system is placed in proximity of the therapeutic target thus requiring only a fraction of the systemic dose. In yet another embodiment, a controlled release system is placed in proximity of the therapeutic target, thus requiring only a fraction of the systemic. Other controlled release systems are discussed in the review by Langer (1990). Science 249:1527-1533.

In another embodiment, the compositions or pharmaceutical compositions can be part of a delayed-release formulation. Delayed-release dosage formulations can be prepared as described in standard references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6th Edition, Ansel et al., (Media, PA: Williams and Wilkins, 1995). These references provide information on excipients, materials, equipment and process for preparing tablets and capsules and delayed release dosage forms of tablets, capsules, and granules. These references provide information on carriers, materials, equipment and process for preparing tablets and capsules and delayed release dosage forms of tablets, capsules, and granules.

As described herein, methods of preparing such pharmaceutical compositions are known, or will be apparent upon consideration of this disclosure, to those skilled in the art. See, e.g., Remington's Pharmaceutical Sciences, Mack Publishing Company, Easton, Pennsylvania, 17th edition, 1985. The composition or formulation to be administered will, in any event, contain a quantity of the composition or pharmaceutical composition adequate to achieve the desired state in the subject being treated.

The compositions can be a component of a pharmaceutical formulation. The pharmaceutical formulation can further contain known agents for the treatment of diseases such as cancer, or symptoms thereof.

Embodiments also provides packaged composition(s) or pharmaceutical composition(s) for prevention, restoration, or use in treating the disease or condition. Other packaged compositions or pharmaceutical compositions can further include indicia including at least one of: instructions for using the composition to treat the disease or condition. The kit can further include appropriate buffers and reagents known in the art for administering various combinations of the components listed herein to the host.

In embodiments, a therapeutically effective amount of the compositions described herein can be administered to the subject. The term “therapeutically effective amount” can refer to that amount of a composition or pharmaceutical composition being administered that will relieve to some extent one or more of the symptoms of the disease or condition being treated, and/or that amount that will prevent, or that will prevent to some extent, one or more of the symptoms of the condition or disease that the subject being treated has or is at risk of developing. In an embodiment, therapeutically effective amount can refer to an amount needed to treat cancer, such as a solid cancer or a non-solid cancer, or at least one pathological effect resulting from the presence of a cancerous condition in a subject human or animal.

In embodiments, a therapeutically effective amount of the compositions described herein can comprise less than about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

In embodiments, a therapeutically effective amount of the compositions described herein can comprise less than 0.01 mg/kg, about 0.01 mg/kg, about 0.05 mg/kg, about 0.1 mg/kg, about 0.1 mg/kg, about 0.5 mg/kg, about 1.0 mg/kg, about 2.5 mg/kg, about 5 mg/kg, about 7.5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 70 mg/kg, about 80 mg/kg, about 90 mg/kg, about 100 mg/kg, about 120 mg/kg, about 135 mg/kg, about 150 mg/kg, about 175 mg/kg, about 200 mg/kg, about 225 mg/kg, about 250 mg/kg, about 275 mg/kg, about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 525 mg/kg, about 550 mg/kg, about 575 mg/kg, about 600 mg/kg, about 625 mg/kg, about 650 mg/kg, about 675 mg/kg, about 700 mg/kg, about 725 mg/kg, about 750 mg/kg, about 775 mg/kg, about 800 mg/kg, about 825 mg/kg, about 850 mg/kg, about 875 mg/kg, about 900 mg/kg, about 1.0 g/kg, about 1.5 g/kg, about 2.0 g/kg, about 2.5 g/kg, about 5 g/kg, about 10 g/kg, about 25 g/kg, about 50 g/kg, or more than 50 g/kg of compound per body weight of a subject.

Aspects of the invention are drawn towards a method for identifying a compound therapeutic for cancer cells having a mutant isocitrate dehydrogenase (mutIDH), comprising screening for compounds that activate or hyperactivate the mutIDH in a cell.

Aspects of the invention are drawn towards a method for identifying a compound therapeutic for cancer cells having an isocitrate dehydrogenase 2 (IDH2) that has an amino acid substitution at R140, comprising screening for compounds that facilitate the IDH2 to use NADH to reduce α-ketoglutarate to 2-hydroxyglutarate.

Aspects of the invention are drawn towards a method for identifying a compound therapeutic for cancer cells having isocitrate dehydrogenase 2 (IDH2) that has an amino acid substitution at R172, comprising screening for compounds that facilitate the IDH2 to use NADH to reduce α-ketoglutarate to 2-hydroxyglutarate.

### Kits

Any of the compositions described herein can be comprised in a kit.

Some components of the kits can be packaged in aqueous media or in lyophilized form. The container means of the kits can include at least one vial, test tube, flask, bottle, syringe or other container means, into which a component can be placed, and suitably aliquoted. Where there is more than one component in the kit, the kit also can contain a second, third or other additional container into which the additional components can be separately placed. However, various combinations of components can be comprised in a vial. The kits of the invention also can include a means for containing the components in close confinement for commercial sale. Such containers can include injection or blow molded plastic containers into which the vials are retained.

When the components of the kit are provided in one and/or more liquid solutions, the liquid solution is an aqueous solution, with a sterile aqueous solution being useful. In some cases, the container means can itself be a syringe, pipette, and/or other such like apparatus, from which the formulation can be applied to an infected area of the body, injected into an animal, and/or even applied to and/or mixed with the other components of the kit.

However, the components of the kit can be provided as dried powder(s). When reagents and/or components are provided as a dry powder, the powder can be reconstituted by the addition of a suitable solvent. The solvent can also be provided in another container means. The kits can also comprise a second container means for containing a sterile, pharmaceutically acceptable buffer and/or another diluent.

In embodiments of the invention, cells that are to be used for cell therapy are provided in a kit, and in some cases the cells can be the sole component of the kit. The kit can comprise reagents and materials to make the cell. In embodiments, the reagents and materials include primers for amplifying sequences, nucleotides, suitable buffers or buffer reagents, salt, and so forth, and in some cases the reagents include vectors and/or DNA as described herein and/or regulatory elements therefor.

In embodiments, there are one or more apparatuses in the kit suitable for extracting one or more samples from an individual. The apparatus can be a syringe, scalpel, and so forth.

In embodiments of the invention, the kit, in addition to cell therapy embodiments, also includes a second cancer therapy, such as chemotherapy, hormone therapy, and/or immunotherapy, for example. The kit(s) can be tailored to a particular cancer for an individual and comprise respective second cancer therapies for the individual.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided herein to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

Enzyme Hyperactivation Causes Toxicity from Isocitrate Dehydrogenase

Mutant isocitrate dehydrogenase (IDH) inhibitors suppress production of the oncometabolite 2-hydroxyglutarate (2HG) and induce responses in IDH-mutant cancers¹⁻⁷, but drug resistance can develop following acquisition of secondary IDH mutations⁸⁻¹³. Resistance mutations in the dimer interface of mitochondrial IDH2 exhibit an unusual pattern wherein they occur in trans (on the other allele) relative to the 2HG-producing active-site mutation¹¹. Here, we show that in contrast to the in trans mutations that drive drug resistance, forced expression of a dimer-interface mutation in cis with the active-site mutation resulted in mitochondrial dysfunction and impaired growth of leukemia cells. Biochemical and structural studies indicated that the in cis dimer-interface mutation facilitated IDH2 to use NADH as an additional cofactor and enhanced production of 2HG. Seeking to exploit the toxicity exerted by the in cis dimer-interface mutation, we performed a chemical screen and identified small molecules which can mimic this aberrant enzymatic activity with selective toxicity towards IDH2-mutant leukemia cells. The findings indicate genetic or pharmacologic hyperactivation, rather than inhibition, as an approach to unleash the toxicity of oncogenes for cancer therapy.

Oncogenic gain-of-function mutations in the active site of isocitrate dehydrogenase 1 and 2 (IDH1 and IDH2) facilitate production of the metabolite 2-hydroxyglutarate (2HG), which drives oncogenesis through pleiotropic effects on chromatin, metabolism, and differentiation^{1,2}. Allosteric inhibitors of mutant IDH enzymes bind to the IDH dimer interface, block production of 2HG, and induce differentiation of IDH-mutant cancer cells³⁻⁵. However, despite suppressing 2HG production in IDH-mutant acute myeloid leukemias, mutant IDH inhibitors induce clinical responses in less than half of cases^{6,7}. Even in those cases that respond, acquired resistance to mutant IDH inhibitors develops⁸⁻¹⁰. One mechanism of resistance results from acquisition of secondary mutations at residues within the dimer-interface of IDH1 or IDH2 that disrupt drug binding, thereby rescuing 2HG production by the mutant enzyme¹¹⁻¹³. Secondary mutations in the dimer-interface of cytosolic IDH1 exhibit the canonical pattern for drug-resistance mutations wherein they occur in cis (on the same allele) relative to the active-site mutation at arginine 132 (R132)¹⁴. This contrasts with an unusual pattern for mitochondrial IDH2 wherein secondary mutations in the dimer-interface were identified in trans (on the other allele) relative to the active-site mutation R140Q. We describe herein the biologic basis for the in trans allelic configuration for dimer-interface mutations in mutant IDH2.

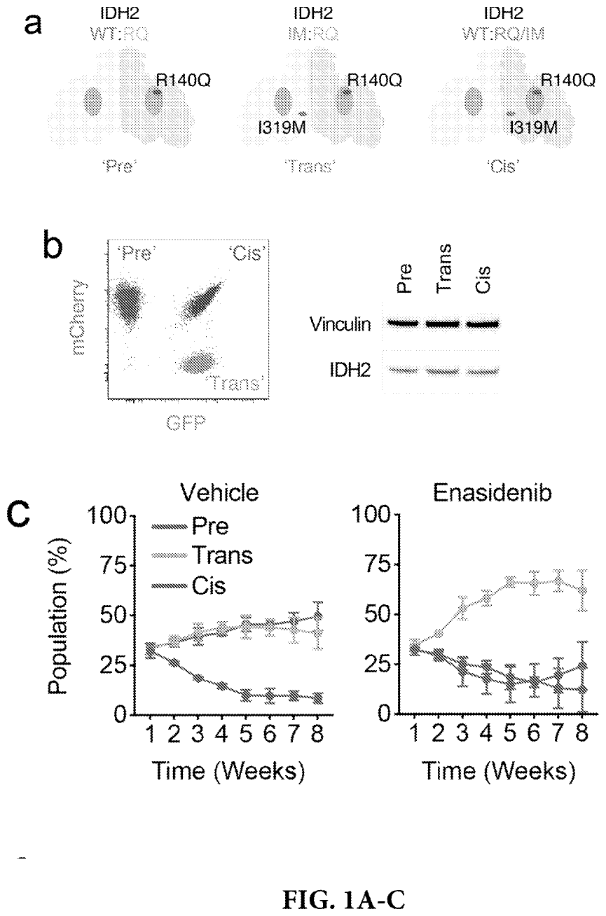

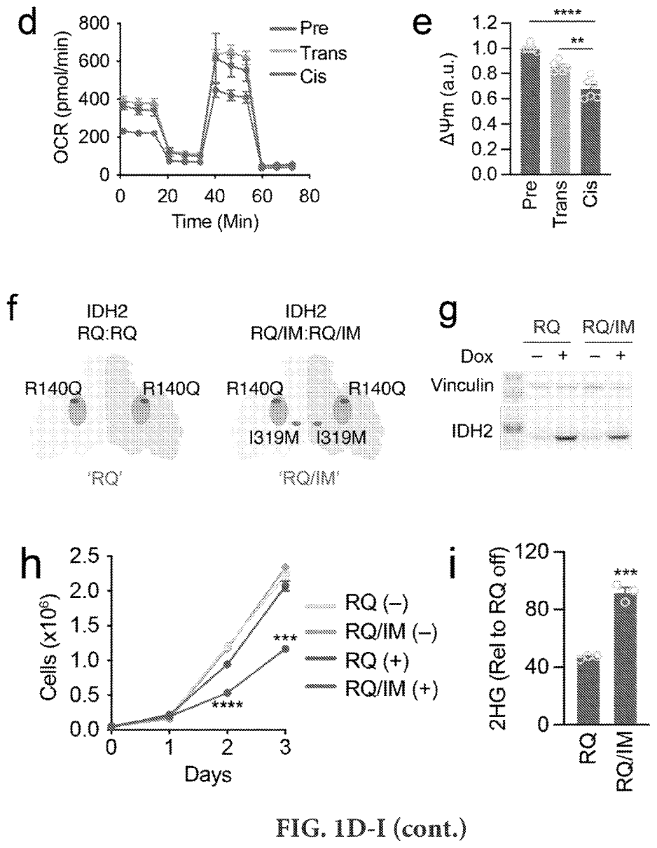

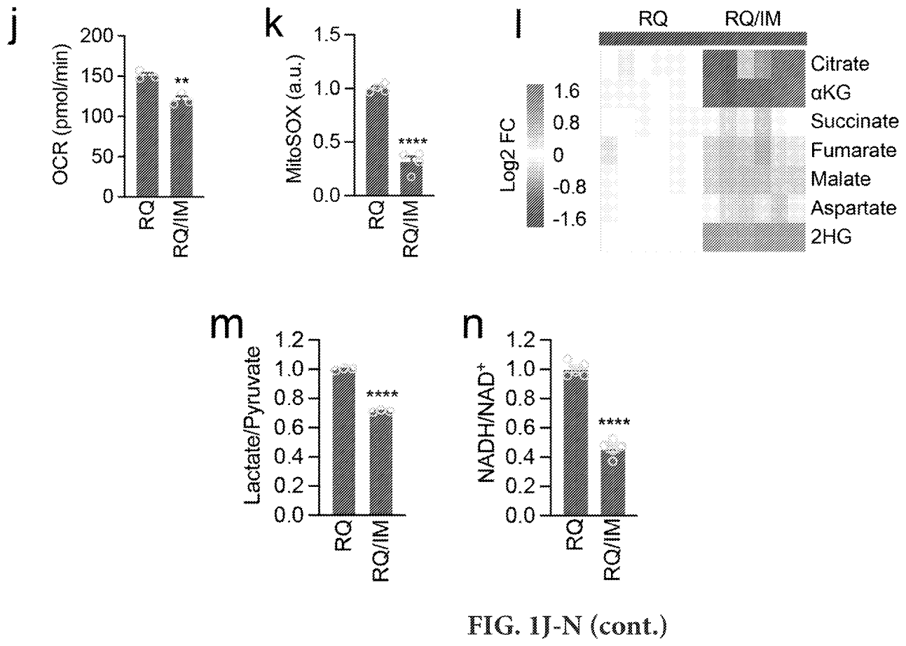

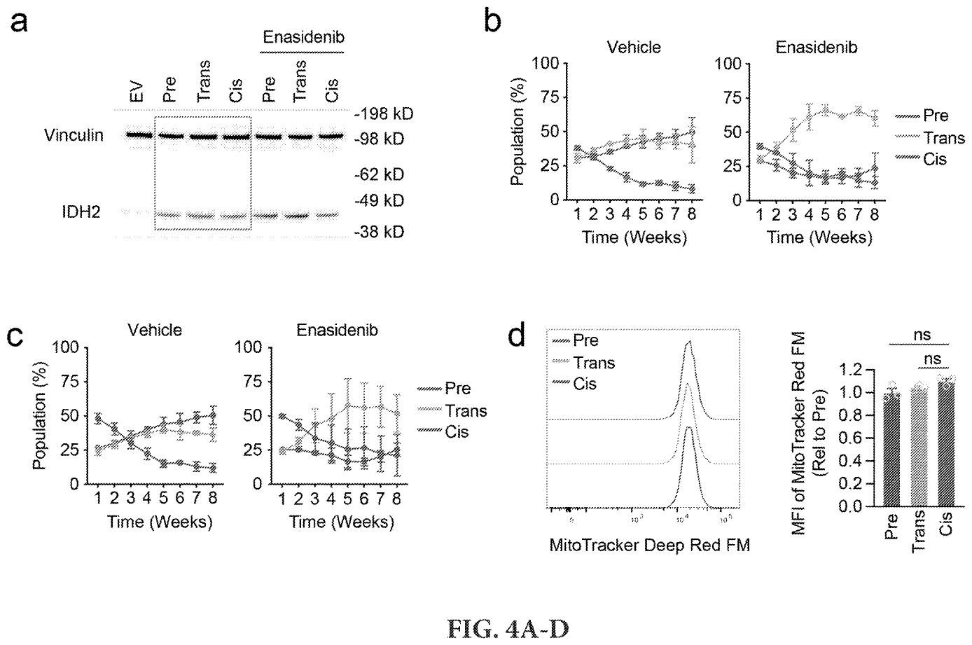

Without wishing to be bound by theory, there can be detrimental effects on cellular fitness that result from expression of an in cis dimer-interface mutation in mitochondrial IDH2. We used retroviral co-infection to generate Ba/F3 leukemia cells with stable expression of each of the IDH2 allelic configurations, including ‘Pre’ (pre-treatment configuration with co-expression of IDH2 wildtype and R140Q alleles), ‘Trans’ (trans configuration observed in drug-resistant patients with co-expression of IDH2 R140Q and I319M alleles), and ‘Cis’ (cis configuration not observed in patients with co-expression of IDH2 wildtype and R140Q/I319M alleles) (FIG. 1 panel A; FIG. 4 panel A). Cells harboring these distinct allelic configurations were marked with different combinations of fluorescent proteins (FIG. 1 panel B). In competitive growth cultures, cells expressing the Cis allelic configuration exhibited a selective growth disadvantage both in the absence and presence of the mutant IDH2 inhibitor enasidenib, whereas cells expressing the Trans configuration selectively grew out in the presence of enasidenib (FIG. 1 panel C). The competitive disadvantage of cells expressing the Cis configuration cannot be overcome by increasing the fraction of Cis cells in the mixed cultures (FIG. 4 panels B, C). Cells with Cis configuration exhibited a decreased oxygen consumption rate (OCR) (FIG. 1 panel D) and lower mitochondrial membrane potential (FIG. 1 panel E; FIG. 4 panel D), indicating that the in cis dimer-interface mutation disrupts leukemia cell fitness by impairing mitochondrial function.

To evaluate acute metabolic effects of the in cis dimer-interface mutation, we used lentiviral infection to generate Ba/F3 leukemia cells with doxycycline-inducible expression of IDH2 R140Q (‘RQ’; active-site mutation alone) or IDH2 R140Q/I319M (‘RQ/IM’; active-site mutation with an in cis dimer-interface mutation) (FIG. 1 panels F, G). In the absence of doxycycline, there was no difference in cell proliferation (FIG. 1 panel H). However, doxycycline-induced expression of the cis-mutant RQ/IM significantly impaired cell proliferation compared to expression of the active site-mutant RQ (FIG. 1 panel H). Notably, expression of the cis-mutant RQ/IM resulted in higher levels of intracellular 2HG than the active site-mutant RQ (FIG. 1 panel I). Cells expressing the cis-mutant RQ/IM exhibited a decreased oxygen consumption rate (OCR) (FIG. 1 panel J) and lower mitochondrial reactive oxygen species (ROS) (FIG. 1 panel K) compared to cells expressing the active site-mutant RQ. We further observed that expression of the cis-mutant RQ/IM resulted in a collapse of TCA cycle metabolite pools (FIG. 1 panel L), accompanied by decreases in the lactate/pyruvate (FIG. 1 panel M) and NADH/NAD⁺ (FIG. 1 panel N) ratios. Collectively, these findings indicate that the IDH2 cis-mutant RQ/IM results in metabolic dysfunction and impaired cell growth, which can explain why the cis allelic configuration has not been observed as a mechanism of drug resistance to mutant IDH2 inhibitors in patients.

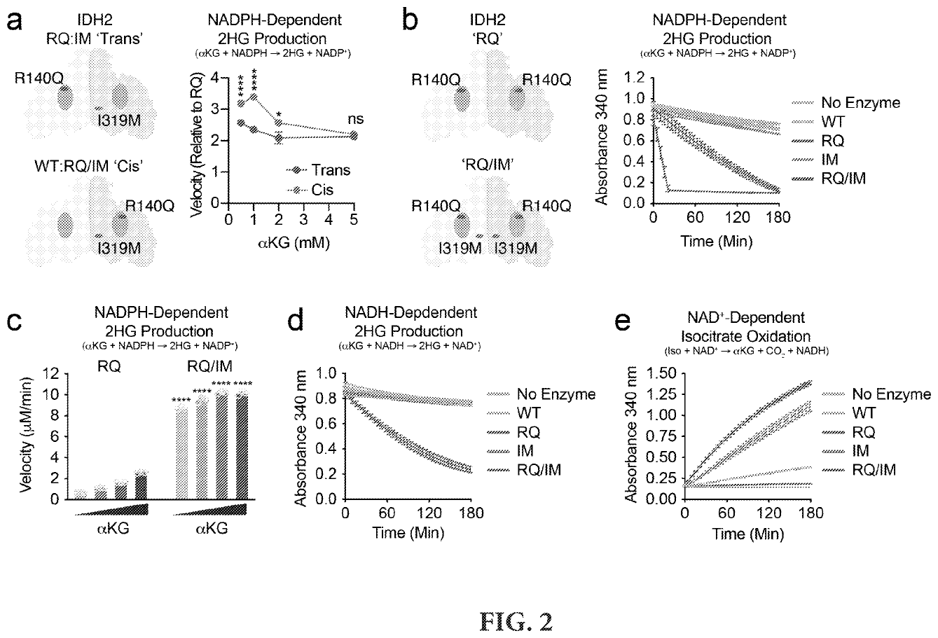

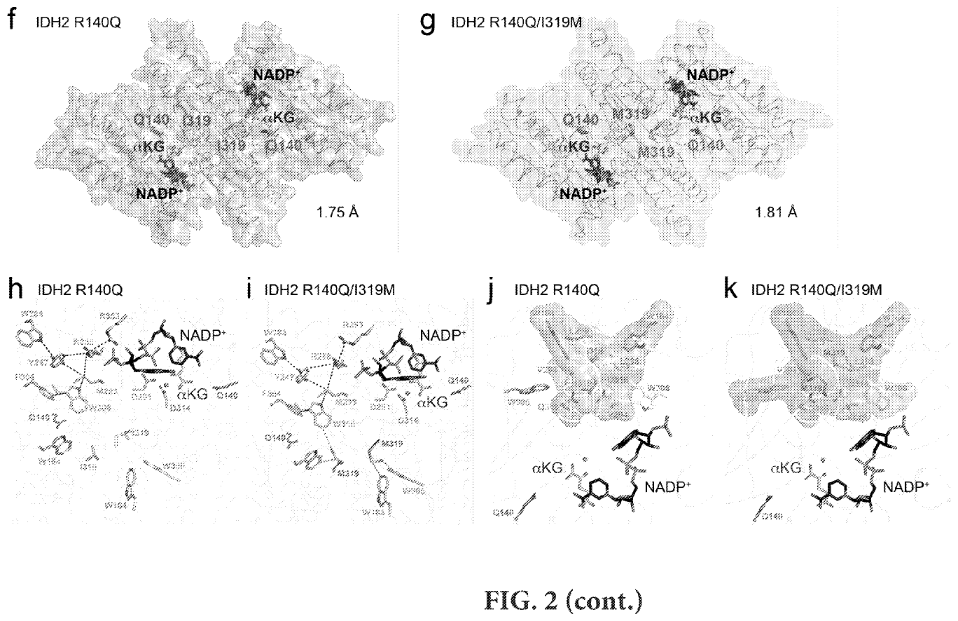

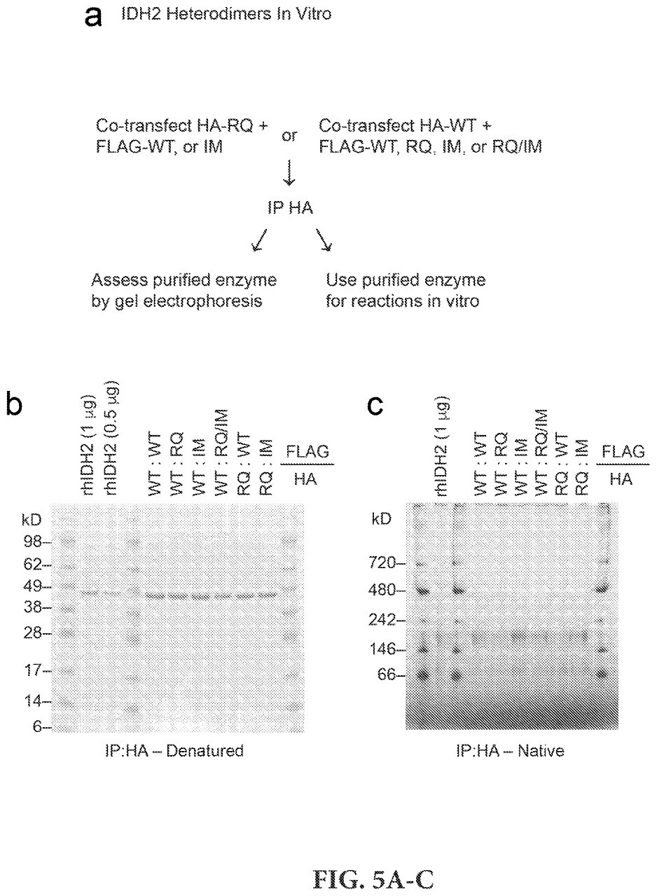

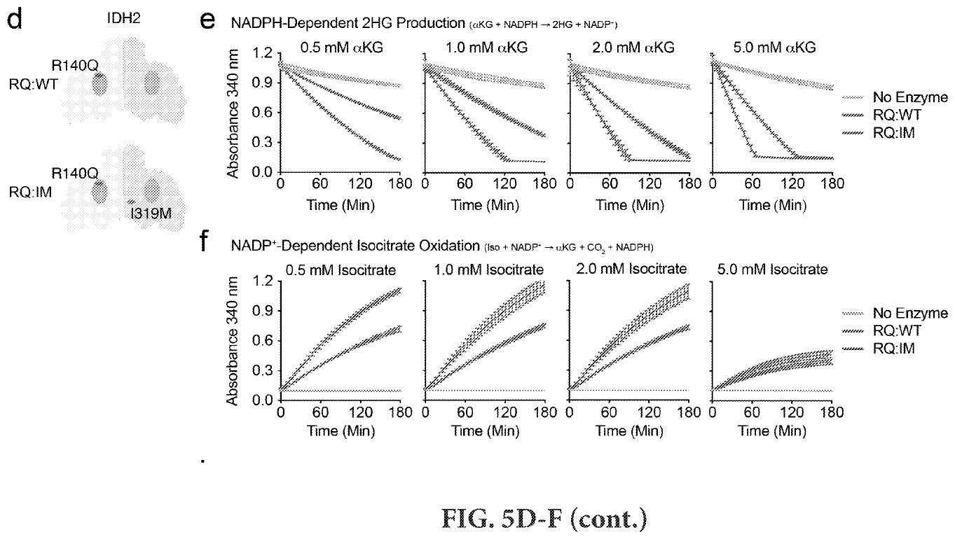

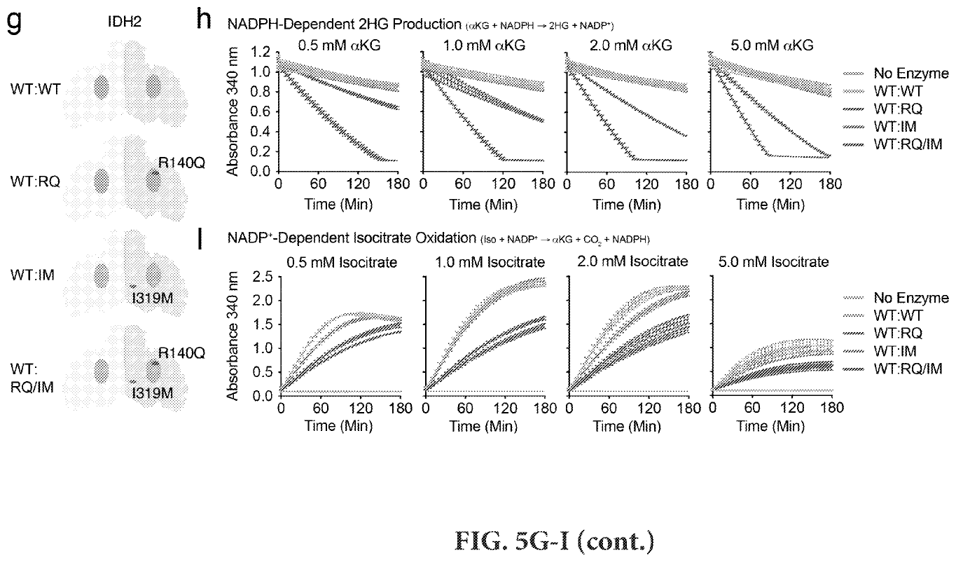

We can determine whether differential effects on IDH2 enzyme function can account for the metabolic dysfunction observed in cells expressing the cis-mutant RQ/IM compared to cells expressing the active site-mutant RQ. To interrogate the biochemical effects of the allelic configuration on IDH2 enzyme function, we purified IDH2 heterodimers harboring the R140Q active-site mutation in trans or in cis with the I319M dimer-interface mutation (FIG. 2 panel A; FIG. 5 panels A-C). Compared to conventional WT:RQ heterodimers, RQ:IM heterodimers with the RQ and IM mutations in trans (FIG. 5 panel D) exhibited enhanced NADPH-dependent 2HG production (FIG. 2 panel A; FIG. 5 panel E) but defective isocitrate oxidation (FIG. 5 panel F). In contrast, WT:RQ/IM heterodimers harboring the RQ and IM mutations in cis (FIG. 5 panel G) showed more enhancement of 2HG production (FIG. 2 panel A; FIG. 5 panel H) with modest defects in isocitrate oxidation (FIG. 5 panel I). WT:IM heterodimers harboring the dimer-interface I319M mutation alone (without the active-site R140Q mutation) displayed no production of 2HG (FIG. 5 panel H) but impaired oxidative decarboxylation of isocitrate (FIG. 5 panel I). These finding indicate that the I319M dimer-interface mutation selectively alters mutant IDH2 enzyme function to enhance reductive 2HG production and impair oxidative isocitrate decarboxylation.

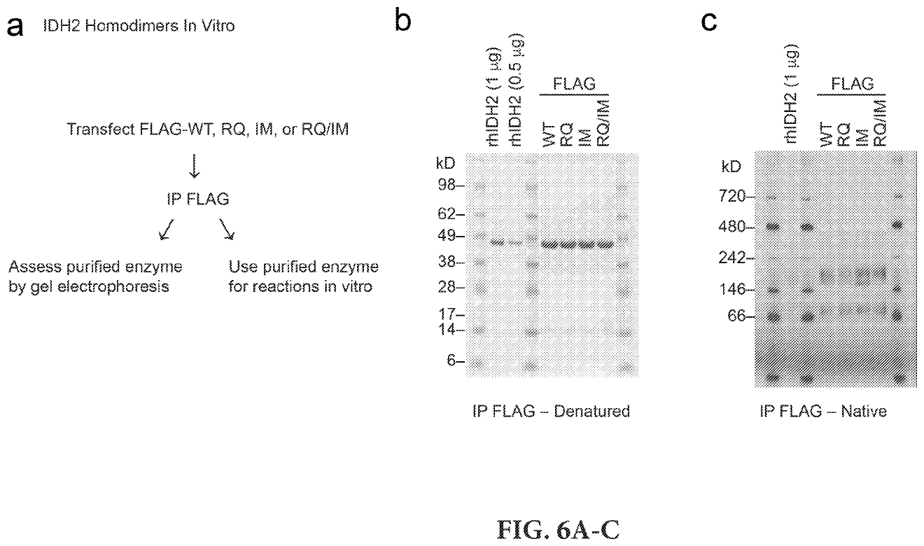

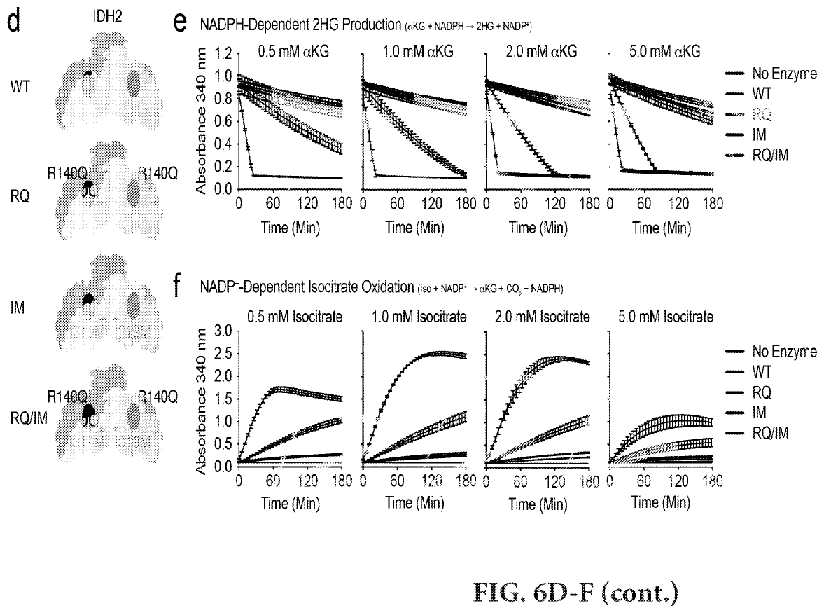

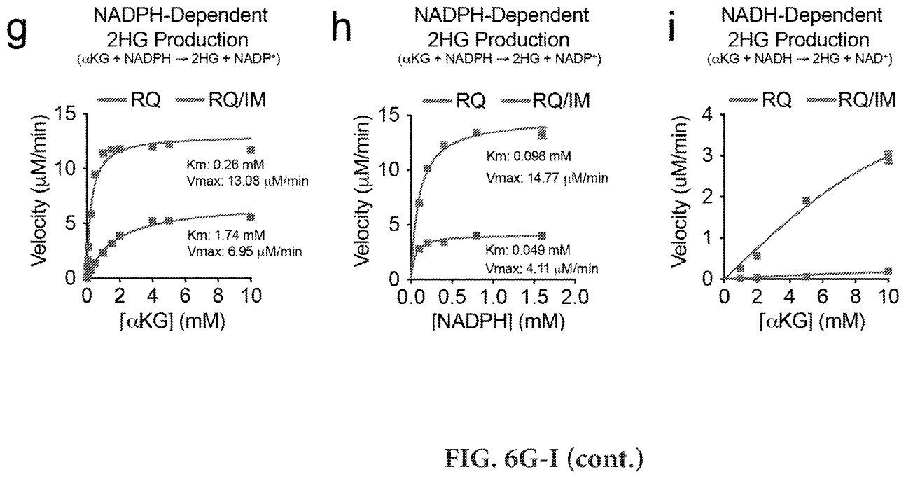

Biochemical effects were evident in IDH2 homodimers (FIG. 6 panels A-F), where we observed synergistic enhancement of 2HG production by combining the R140Q active-site mutation in cis with the I319M dimer-interface mutation (FIG. 2 panels B, C). Relative to active-site RQ homodimers, cis-mutant RQ/IM homodimers exhibited a decreased Kₘ for αKG (0.26 mM for RQ/I4 compared to 1.74 mM for RQ), as well as an increased Vₘₐₓ for NADPH-dependent 2HG production (13.08 μM/min for RQ/IM compared to 6.95 μM/min for RQ) (FIG. 2 panel C; FIG. 6 panels G, H). Calculations of enzyme efficiency confirmed that cis-mutant RQ/IM homodimers were better at catalyzing NADPH-dependent 2HG production compared to active-site RQ homodimers (k_{cat}/Kₘ=2.75×10⁷ M-1s⁻¹ for RQ/IM compared to k_{cat}/Kₘ=2.14×10⁶ M⁻¹s⁻¹ for RQ). IDH2 homodimers harboring the dimer-interface I319M mutation alone (without the active-site R140Q mutation) had an ability to produce 2HG (FIG. 2 panel b; FIG. 6 panel E) and defective isocitrate oxidation (FIG. 6 panel F).

We also found that cis-mutant RQ/IM homodimers gained the ability to aberrantly use NAD(H) as a cofactor. Whereas conventional active-site RQ homodimers had no ability to use NADH to reduce αKG, cis-mutant RQ/IM homodimers were able to perform NADH-dependent reduction of αKG to 2HG (FIG. 2 panel D; FIG. 6 panel I). Moreover, the I319M dimer-interface mutation, alone or in combination with the active-site R140Q mutation, enhanced the capacity for NAD⁺-dependent isocitrate oxidation (FIG. 2 panel E). Collectively, these findings indicate that the in cis dimer-interface mutation I319M dramatically alters IDH2 enzyme function, substantially enhancing NADPH-dependent 2HG-production, in addition to enabling aberrant utilization of NAD(H) for 2HG production and isocitrate oxidation. The abnormal enzymatic function of the cis-mutant enzyme can result in mitochondrial dysfunction, redox imbalance, and metabolic derangements that impede cell growth (FIG. 1).

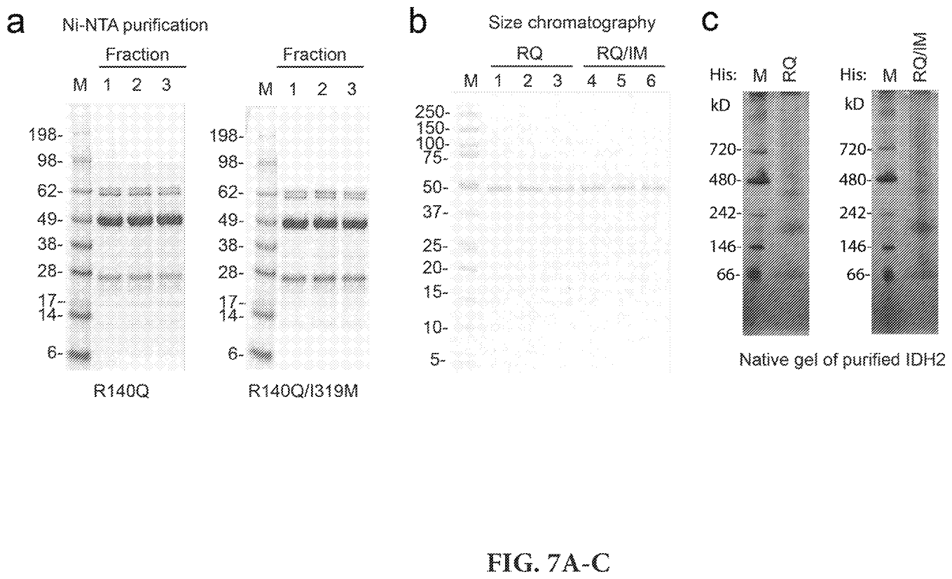

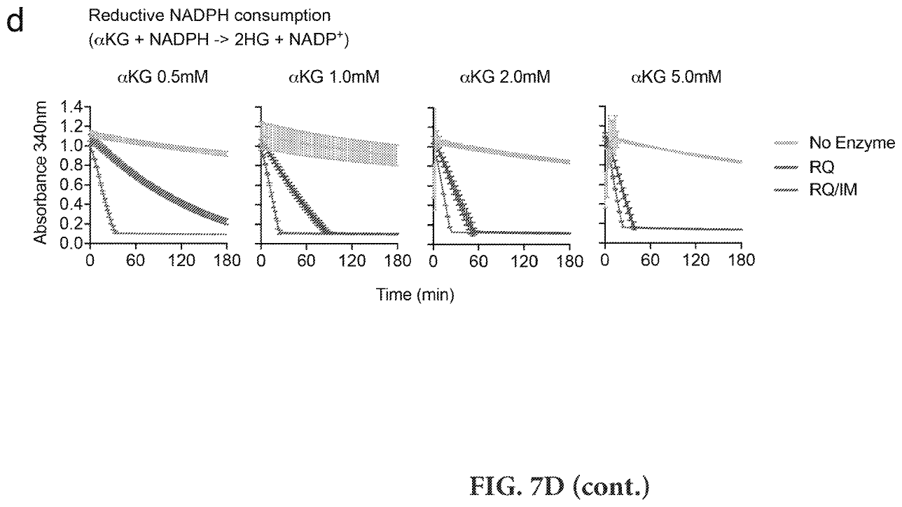

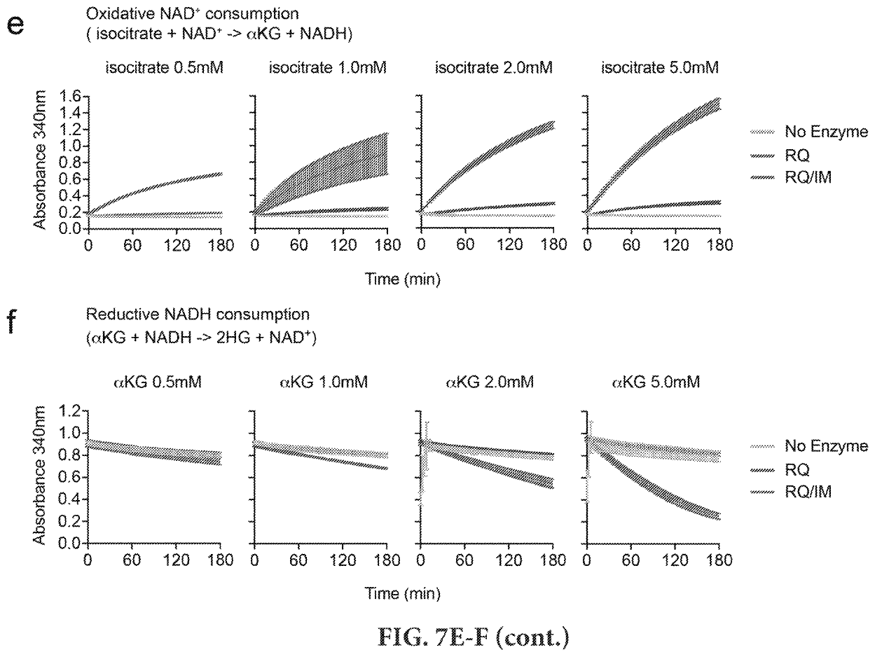

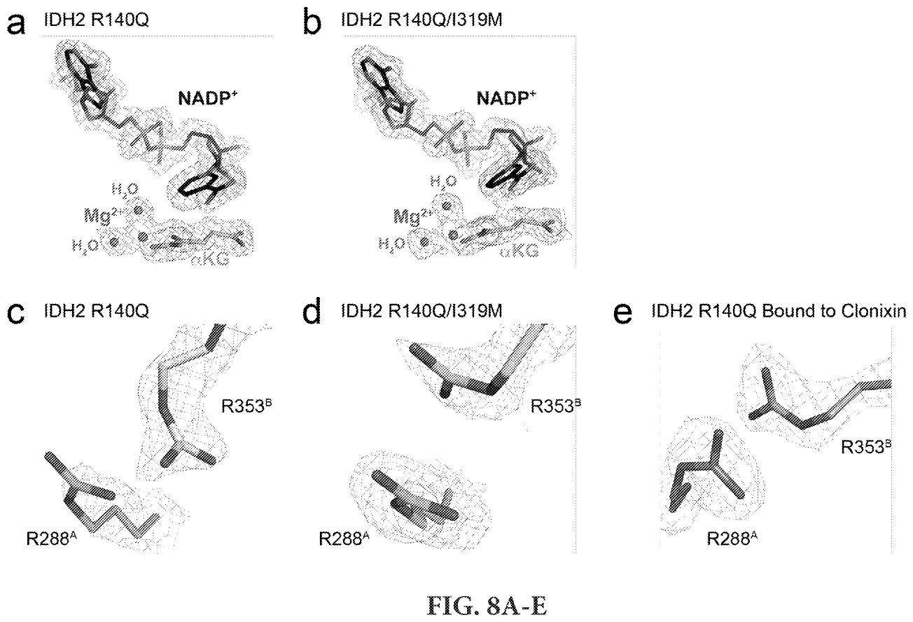

We can interrogate the structural basis by which the in cis dimer-interface mutation I319M alters IDH2 enzymatic activity. We expressed the IDH2 R140Q (RQ) and R140Q/I319M (RQ/IM) proteins in bacteria and purified homodimers by sequential Ni-NTA column and size-exclusion chromatography (FIG. 7 panels A-F). We determined crystal structures of RQ at 1.75 Å resolution (FIG. 2 panel F) and RQ/IM at 1.81 Å resolution (FIG. 2 panel G) in Michaelis complex with NADP⁺, αKG, and Mg²⁺, using identical crystallization conditions to avoid structural bias (Table 2 and Table 3; FIG. 8 panels A, B). In contrast to weak and disordered hydrophobic interactions observed between the native isoleucine 319 (I319) and W164 (4.5 Å) and W306 (6.1 Å) in RQ (FIG. 2 panels F, H), the sulfur of methionine 319 (M319) in RQ/IM formed stronger S-π interactions with W164 (3.8 Å), while its methyl group formed hydrophobic interactions with W306 (4.0 Å) in each subunit of the dimer (FIG. 2 panels G, I)¹⁵. Moreover, conformational differences present in RQ/IM shifted the positions of R288 from subunit A and R353 from subunit B, resulting in more favorable π-π interactions across the dimer-interface (FIG. 2 panel I; FIG. 8 panels C, D)¹⁶. The distinct interactions between M319 and the surrounding residues resulted in an increased buried surface area at the dimer interface of RQ/IM (1,448 Å2) compared to RQ (1,415 Å2) (FIG. 2 panels J, K), as calculated by CNS-1.317. Taken together, these findings indicate that the I319M mutation strengthens molecular interactions within the IDH2 dimer-interface, analogous to a report on the effects of a S280F mutation in IDH118. The enhanced strength of dimerization in RQ/IM can account for its enhanced catalytic function and ability to use the less optimal cofactor NAD(H) for catalysis, whereas the conventional RQ enzyme strictly requires NADP(H).

TABLE 2
Data collection and refinement statistics for human IDH2 R140Q (‘RQ’) and
R140Q/1319M (RQ/IM).

| Protein | RQ | RQ/IM | RQ |
|---|---|---|---|
| Data collection | | | |
| Ligands | NADP⁺, Mg²⁺, αKG | NADP⁺, Mg²⁺, αKG | NADP⁺, Clonixin |
| Crystallization reagent # | 1 | 1 | 2 |
| Space group | C222₁ | P3₁21 | P1 |
| Cell dimensions | | | |
| a, b, c (Å) | 66.1, 140.5, 94.9 | 100.1, 100.1, 144.1 | 67.5, 78.7, 103.2 |
| a, [symbol] , ψ (°) | 90, 90, 90 | 90, 90, 120 | 78.8, 77.2, 70.0 |
| Resolution (Å) | 94.95-1.75 | 144.04-1.81 | 99.7-2.24 |
| | (1.78-1.75)* | (1.85-1.81)* | (2.28-2.24)* |
| R_{merge} (%) | 7.3 (158) | 16.0 (232) | 19.2 (119) |
| I/0I | 21.1 (1.7) | 16.7 (1.3) | 5.0 (1.2) |
| Completeness (%) | 99.8 (96.8) | 99.6 (93.4) | 93.2 (90.7) |
| Redundancy | 13.4 (12.6) | 20.1 (17.5) | 3.7 (3.4) |
| CC1/2 | 1.00 (0.67) | 1.00 (0.41) | 0.98 (0.33) |
| Refinement | | | |
| | 59.80-1.79 | 86.68-1.88 | 62.49-2.50 |
| Resolution (Å) | (1.81-1.79)* | (1.90-1.88)* | (2.53-2.50)* |
| No. reflections | 41,985 (4,162) | 68,434 (6,958) | 61,945 (6,173) |
| R_{work}/R_{free} (%) | 15.4 (23.1)/ | 15.2 (23.2)/ | 16.0 (26.90)/ |
| | 17.9 (24.6) | 18.6 (30.5) | 23.2 (32.4) |
| Ramachandran Plot (%) | | | |
| Outliers | 0 | 0 | 0.06 |
| Allowed | 3.16 | 2.98 | 4.77 |
| Favored | 96.84 | 97.02 | 95.17 |
| No. atoms | | | |
| Protein | 3,288 | 6,684 | 13,052 |
| Ligand/ion | 59 | 118 | 264 |
| Water | 248 | 669 | 351 |
| B-factors | | | |
| Protein | 35.8 | 27.9 | 46.1 |
| Ligand/ion | 27.1 | 21.4 | 38.4 |
| Water | 45.5 | 38.5 | 46.3 |
| R.m.s deviations | | | |
| Bond lengths (Å) | 0.007 | 0.007 | 0.008 |
| Bond angles (°) | 0.881 | 0.894 | 0.949 |

*Highest resolution shell is shown in parenthesis.

TABLE 3
Crystallization Conditions

| Crystallization | IDH2 R140Q (RQ) and |
|---|---|
| reagent # | IDH2 R140Q/1319M (RQ/IM) |
| 1 | 100 mM HEPES, pH 7.5, 100 mM |
| | Magnesium Acetate tetrahydrate, |
| | 40% (w/v) PEG 4,000 |
| 2 | 4.3M Sodium chloride, 100 mM HEPES, pH 7.5 |

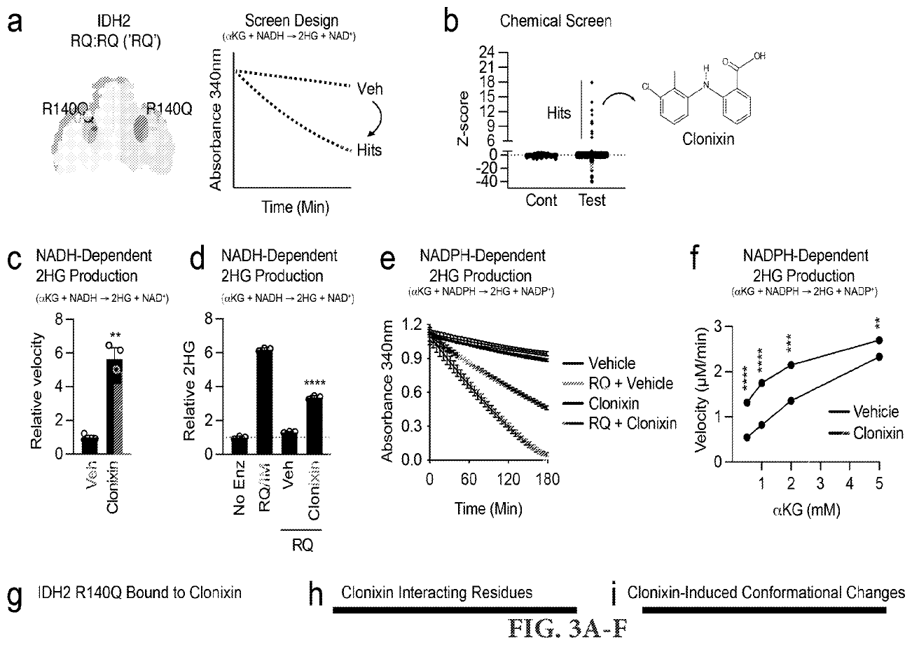

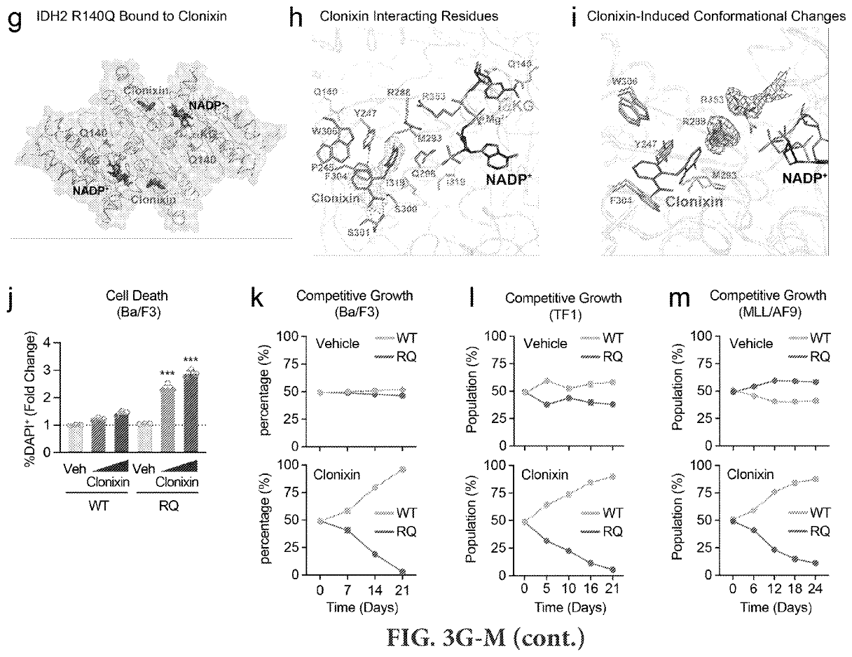

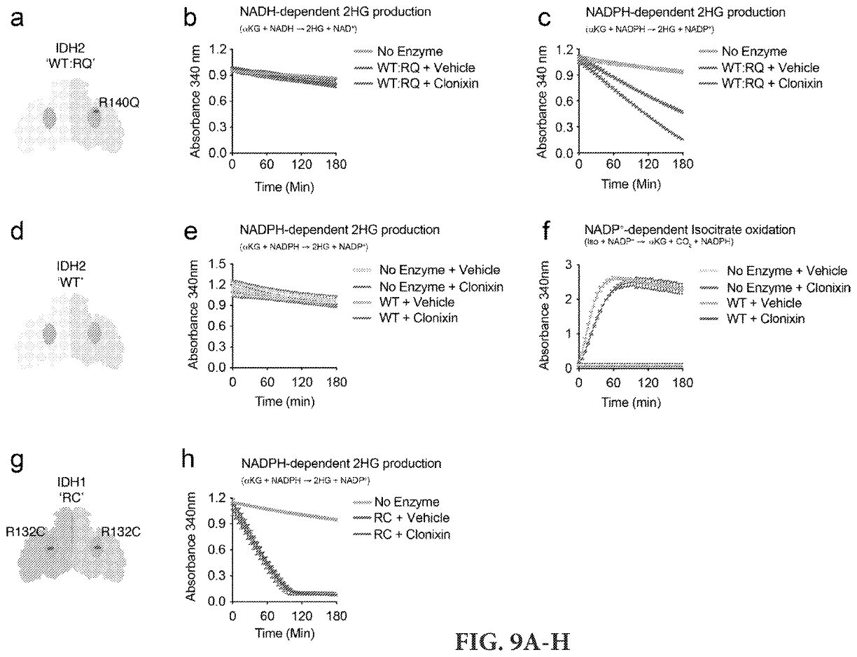

Our findings indicate that the cis-mutant IDH2 RQ/IM enzyme possesses unique structural and biochemical properties that exert metabolic toxicity in leukemia cells. Without wishing to be bound by theory we can identify small molecules which can mimic the cis-mutant activity¹⁹, thereby converting the conventional IDH2 R140Q enzyme into a toxic metabolic liability. To study this, we performed a high-throughput chemical screen to identify small molecules that facilitate IDH2 R140Q to catalyze NADH-dependent reduction of αKG to 2HG (FIG. 3 panel A). The chemical screen exhibited minimal background and identified several compounds with Z-scores greater than 6 (Table 4), with the molecule clonixin being amongst the hits with the strongest activity (FIG. 3 panel B)²⁰. We purchased commercially available clonixin and validated its effects on the mutant IDH2 R140Q enzyme, using enzyme assays with purified enzyme, substrates and cofactors. Like the in cis I319M dimer-interface mutation, clonixin was sufficient to facilitate mutant IDH2 to use NADH to reduce αKG (FIG. 3 panel C; FIG. 9 panels A, B) to 2HG (FIG. 3 panel D). Moreover, clonixin enhanced NADPH-dependent production of 2HG by IDH2 R140Q (FIG. 3 panels E, F; FIG. 9 panels A, C). In contrast, clonixin did not facilitate wildtype IDH2 to produce 2HG and had minimal effect on its ability to perform oxidative decarboxylation of isocitrate to αKG (FIG. 9 panels D-F). Notably, clonixin did not substantially alter the enzymatic activity IDH1 R132H (FIG. 9 panels G, H), indicating that its effects are selective for mutant IDH2.

TABLE 4
Concordant hits from two independent high-throughput screens

| | | Well | Z-Score | |
|---|---|---|---|---|
| Drug Name | Formula | ID | Screen #1 | Screen #2 |
| Clonixin | C13H11CIN2O2 | G11 | 9.71267 | 5.93552 |
| Idebenone | C19H30O5 | A20 | 11.33144 | 8.49858 |
| Dihydrogedunin | C28H36O7 | D20 | 9.57777 | 4.45164 |
| Deoxygedunin | C28H34O6 | J21 | 7.95899 | 7.55430 |
| Gossypetin | C15H10O8 | C12 | 9.98246 | 13.35492 |

To elucidate the structural basis for clonixin activity, we determined a crystal structure of clonixin bound to IDH2 R140Q with NADP⁺, αKG, and Mg²⁺ at 2.5 Å resolution (FIG. 3 panel G). While NADP⁺ and clonixin had well-defined electron densities, αKG and Mg²⁺ did not, and thus were not modeled. The structure indicated that clonixin bound to the periphery of the IDH2 dimer-interface at a site distinct from the central region where the mutant IDH2 inhibitor enasidenib binds³. Specifically, clonixin occupied a small hydrophobic pocket where it interacted with residues Y247, R288, M293, Q296, S300, and S301 through π-π and polar interactions (FIG. 3 panel H). Analogous to the structure of RQ/IM (FIG. 2 panel I), clonixin binding repositioned R288 of subunit A and R353 of subunit B to enhance π-π stacking across the dimer-interface (FIG. 3 panel I; FIG. 8 panel E). The network of interactions mediated by clonixin caused the two subunits of IDH2 to move closer to one another by approximately 0.4 Å, as measured by the distance between two I319-Cα atoms. Taken together, these findings indicate that clonixin functions as a molecular glue that strengthens intermolecular interactions within the dimer-interface of IDH2, analogous to the effects of the I319M mutation.

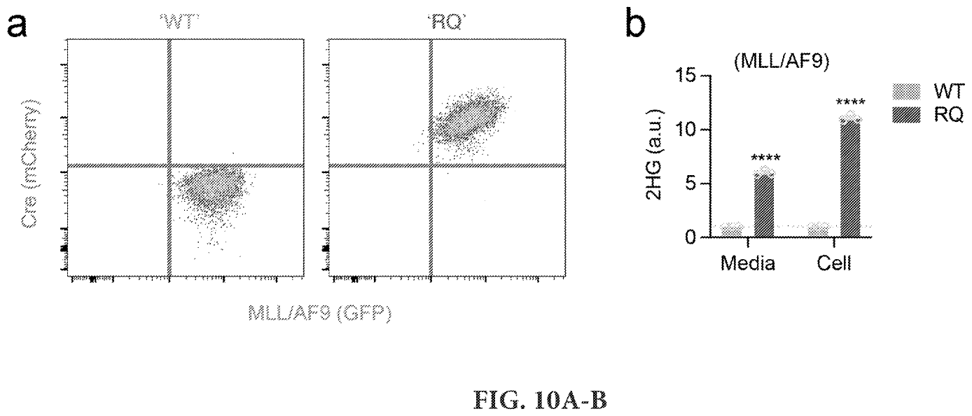

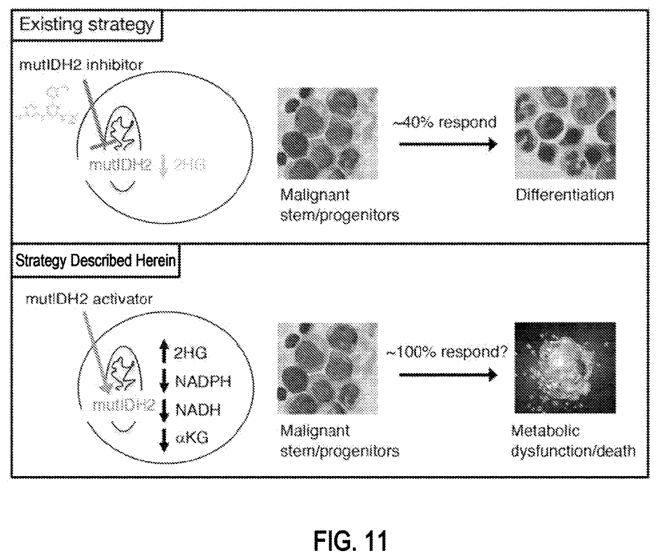

We next studied whether clonixin can exert selective toxicity towards IDH2-mutant leukemia cells. We used retroviral transduction to generate Ba/F3 cells with stable expression of wildtype IDH2 or mutant IDH2 R140Q. Upon treatment with clonixin in vitro, Ba/F3 cells expressing IDH2 R140Q, but not wildtype IDH2, exhibited increased cell death (FIG. 3 panel J) and progressive elimination from competitive growth cultures (FIG. 3 panel K). As an alternative model, we used isogenic human TF1 leukemia cells which express wildtype IDH2 or mutant IDH2 R140Q from the endogenous IDH2 locus. Clonixin treatment selectively eliminated the IDH2-mutant TF1 cells from competitive growth cultures (FIG. 3 panel L). Finally, to examine the effect of clonixin on primary leukemia cells, we utilized a mouse model harboring Idh2^{R140Q} in the endogenous Idh2 locus preceded by lox-STOP-lox cassette²¹. Bone marrow hematopoietic stem/progenitor cells were subjected to sequential retroviral transduction with the MLL/AF9 oncogene and then Cre to activate the Idh2^{R140Q} allele (FIG. 10 panels A, B). In the absence of drug, the Idh2-mutant leukemia cells exhibited a slight competitive advantage, whereas clonixin treatment eliminated the Idh2-mutant cells (FIG. 3 panel M).

By investigating an unusual pattern of drug-resistance mutations in patients, we uncovered unexpected insights into the regulation of oncogenic IDH2 activity. Depending on its allelic configuration, the single point mutation I319M within the dimer-interface of IDH2 conferred dramatic effects on catalytic activity, selectively enhancing 2HG production and enabling neomorphic use of NAD(H) as a cofactor. The aberrant enzymatic activity of cis-mutant IDH2 caused metabolic dysfunction and impaired proliferation of leukemia cells. Importantly, this altered enzyme activity can be recapitulated with the small molecule clonixin, which functioned like a molecular glue to strengthen interactions at the IDH2 dimer-interface, similar to the I319M mutation. The cellular toxicity exerted by cis-mutant RQ/IM and clonixin can arise from a combination of excessive 2HG production and consumption of NADPH, NADH, and αKG within the mitochondria. Indeed, accumulating evidence indicates that IDH mutations can exert toxic effects on cells, through 2HG itself or through the aberrant function of the IDH-mutant enzyme²²⁻²⁶. Given that the majority of IDH-mutant tumors are impervious to 2HG inhibition^{6,7,12,27}, the strategy of unleashing metabolic toxicity from the IDH mutant enzyme can be induce responses in more IDH-mutant tumors.

### Methods

### Antibodies and Chemicals

The following antibodies were used: anti-FLAG (Sigma; F1804; clone M2; mouse; 1:1,000), anti-HA (Cell Signaling Technology; 2367S; clone 6E2; mouse; 1:1,000), anti-IDH2 (Abcam; ab55271; clone 5F11; mouse; 1:1,000), anti-vinculin (Cell Signaling Technology; 4650; rabbit; 1:1,000), anti-OXPHOS cocktail (Abcam; ab110413, mouse; 1:1,000), HRP-conjugated secondary anti-mouse (Cytiva; NA931V; sheep; 1:5,000), HRP-conjugated secondary anti-rabbit (Cytiva; NA934V; donkey; 1:5,000). The following chemicals were used: enasidenib (Med Chem Express, HY-18690), clonixin (Cayman; 22284), NAD⁺ (Sigma; N0632), NADH (Sigma; N8129), NADP⁺ (Sigma; N5755), NADPH (Sigma; N7505), α-ketoglutaric acid (Sigma; K2010), and (+)-Potassium Ds-threo-isocitrate monobasic (Sigma; 58790).

### Cell Culture

Cell lines were maintained at low passage number and split every 2-3 days before reaching confluence. HEK293T (ATCC), Platinum-E (Cell Biolabs), and Phoenix-Eco (ATCC) cells were maintained in DMEM with 10% FBS, glucose 25 mM, glutamine 4 mM, penicillin 100 U/ml, and streptomycin 100 μg/ml. The mouse hematopoietic leukemia line, Ba/F3 (Creative Bioarray), was maintained in RPMI 1640 containing 10% fetal bovine serum (FBS), murine IL-3 at 3 ng/mL (R&D Systems), penicillin 100 U/ml, and streptomycin 100 μg/ml. IDH2^{WT}- and IDH2^{R140Q}-mutant TF-1 cells (ATCC) were grown in RPMI with 10% FCS and recombinant human GM-CSF at 2 ng/ml (R&D Systems). Cells were cultured at 37° C. and 5% CO₂ unless otherwise specified. For hypoxia experiments, cells were cultured in a hypoxia chamber (Coy) with 5% CO₂ and 1% 02. Human cell lines were authenticated using ATCC fingerprinting or short tandem repeat (STR) profiling assay at the MSKCC Integrated Genomics Operation Core. Cell lines were confirmed to be negative for mycoplasma infection throughout the experimental period. For competitive growth assays, transduced cells were mixed at the indicated ratios then cultured with vehicle (DMSO), enasidenib, or clonixin. The percentages of each cell population were tracked over time by flow cytometry.

### DNA Constructs

IDH2 and IDH1 DNA constructs with C-terminal HA or FLAG tags were cloned by standard site-directed mutagenesis (Agilent) and Gibson Assembly (New England Biolabs) into pCDNA3.1 (Thermo Fisher), pET-22b(+) (Sigma), MSCV-IRES-mCherry (Addgene), MSCV-IRES-GFP (Addgene), MSCV-IRES-YFP (Addgene), or pCW57-GFP-1 Å-MCS (Addgene). pLenti-mCherry-Cre-blast was purchased from Addgene (179390). MSCV-MLL/AF9-IRES-GFP was a gift from Dr. Omar Abdel-Wahab. Plasmids were verified by Sanger sequencing.

### Transfection, Virus Packaging and Transduction

For transient transfection experiments, 293T cells were transfected using polyethylenimine (PEI). Retroviruses were generated by the co-transfection of cDNA-expressing viral vectors with the packaging vector pCMV-VSVG (Addgene) into platE cells using PEI. Lentivirus were generated by the co-transfection of cDNA-expressing viral vectors with the packaging plasmids psPAX2 and pCMV-VSVG into 293T cells using PEI. ProFection® mammalian transfection system (Promega) was used to generate MLL/AF9 retroviruses according to the manufacturer's instructions. Virus-containing supernatants were cleared of cellular debris by 0.45-μm filtration and mixed with 8 μg/ml polybrene. Target cells were exposed to viral supernatants for spin infection at 32° C. and 2300 rpm for 90 min. After overnight growth, fresh medium was applied, followed by growth for an additional 2 days. Cells were treated with puromycin 1 μg/mL (Gibco; A11138-03) or blasticidin 5 μg/mL (Gibco; A11139-03) for drug selection of transduced cells. For constructs with fluorescent markers, cells were FACS-sorted on a BD Aria 7 or Sony SH800s for selection of transduced cells.

### Retroviral Transduction of Primary Hematopoietic Cells

Animal procedures were conducted in accordance with the Guidelines for the Care and Use of Laboratory Animals and were approved by the Institutional Animal Care and Use Committees (IACUC) at Memorial Sloan Kettering Cancer Center. In brief, ~12-week-old C57BL/6 Idh2^{R140Q+/-} mice (27) were treated with a single dose of 5-flurouracil (0.15 mg/g) followed by bone marrow (BM) cells harvest 5 days after treatment. Red blood cells were lysed and nucleated cells were sequentially infected with retroviruses encoding MSCV-MLL/AF9-IRES-GFP followed by MSCV-Cre-IRES-mCherry. Transduced cells were sorted based on GFP and mCherry expression, then maintained in IMDM with 10% FCS, SCF at 20 ng/ml, IL-3 at 10 ng/ml, and IL6 at 10 ng/ml.

### Flow Cytometry and Flow Sorting

Flow cytometry was performed on BD Fortessa, Guava easyCyte or Cytek 5 Laser Aurora analyzers. FACS sorting was conducted on BD Aria 7 or Sony H800s sorters. Flow cytometry analyses were performed using FlowJo 10.8.1 software.

### Measurement of Oxygen Consumption Rate

Oxygen consumption rate (OCR) was measured using a XFe96 Extracellular Flux Analyzer (Agilent). Seahorse microplates (Agilent) were coated with 22.4 μg/mL Cell-Tak (Corning; 354240) in 0.1 M sodium bicarbonate according to the manufacturer's instructions. Cells were washed with and then plated in prewarmed XF base medium (DMEM with glucose 10 mM, glutamine 2 mM, sodium pyruvate 1 mM, pH 7.4) in Seahorse microplates (Agilent) at fixed concentrations (30,000 or 40,000 cells/well). After plating, cells were centrifuged at 300 g for 1 min and incubated for 45 min at 37° C. in an CO₂-free incubator. OCR analysis was performed at baseline and after injection of oligomycin (1 μM), FCCP (1 μM), or rotenone plus antimycin mix (each 0. μM) according to the manufacturer's instructions. OCR results were analyzed using the Wave software (Agilent) under default settings.

### Mitochondrial ROS Measurement

Mitochondrial superoxide levels were measured by the MitoSox mitochondrial superoxide indicator (Thermo Fisher; M36008) following the recommended manuals. Briefly, cells were harvested and washed with 1×HBSS (without phenol red, with Ca²⁺ Mg²⁺ and glucose; Thermo Fisher; 14025092) one time. Then cells were incubated with 500 μl of 2.5 μM MitoSox reagent in 1×HBSS at 37° C. for 10 minutes in dark. After incubation with dye, cells were centrifuged at 1,700 RPM for 5 mins and washed with 1 mL HBSS by pipetting up and down. Cells were centrifuged and resuspended in HBSS. Fluorescence signals were determined by BD LSRFortessa or Cytek 5 Laser Aurora.

### Mitochondrial Membrane Potential Measurement

Mitochondrial content was assessed using the MitoTracker Deep Red FM probe (Invitrogen) in 25 nM concentration. Mitochondrial membrane potential was examined using MitoProbe™ DilC1(5) (Invitrogen) in 75 nM concentration according to the manufacturer's instructions. Briefly, cells were harvested and washed with prewarmed PBS one time, and then stained for 30 min with MitoTracker Deep Red FM or MitoProbe™ DilC1(5). Cells were then washed again, then fluorescence signals were measured by flow cytometry.

### NADH/NAD⁺ Ratio Measurement

The NADH/NAD+-Glo Assay (Promega) was used according to the manufacturer's instructions. 40,000 cells were used for each assay. The cell lysates were divided for separate measurement of NADH and NAD+. The luminescence was measured using a Microplate Luminometer (Veritas).

### Cell Death Quantification

Ba/F3 cells were seeded in 12-well plates at a density of 250,000 cells/ml. Cells were cultured with vehicle (DMSO) or clonixin at indicated concentration. After 48 hr, cells were harvested, stained with DAPI (0.2 μg/ml) in FACS buffer, and analyzed by flow cytometry.

### Gel Electrophoresis and Western Blotting

For denatured gel electrophoresis, cells were collected and washed with cold PBS, then cell pellets were lysed in RIPA buffer (Cell Signaling) on ice for 20 min, centrifuged at 13,000 rpm at 4° C., and supernatants collected. Protein concentrations were quantified by BCA assay (Thermo Fisher). Protein samples were separated by SDS-PAGE, then stained directly with Coomassie Blue Reagent or transferred to nitrocellulose membranes (Life Technologies). Membranes were blocked with 5% milk in TBST, incubated with primary antibodies overnight at 4° C., then incubated with HRP-conjugated secondary antibodies for 1 hr at room temperature the following day. After incubation with ECL (Thermo Fisher or Kindle Biosciences), digital imaging was performed using the Amersham Imager 800 (Cityva). For native gel electrophoresis, cells were harvested in M-PER buffer (Thermo Fisher). IDH2 enzyme complexes were purified with Pierce Anti-HA Agarose or Anti-FLAG M2 Affinity Gel, separated by NativePAGE (Thermo Fisher), and stained directly with Coomassie reagent.

### Metabolite Measurement by Gas Chromatography-Mass Spectrometry

Metabolites were extracted from cell pellets using ice-cold 80:20 methanol:water solution containing 2 μM deuterated 2-hydroxyglutarate (d-2-hydroxyglutaric-2,3,3,4,4-d5 acid; deuterated-2HG) as an internal standard. After overnight incubation at −80° C., cell extract was collected and centrifuged at 21,000 g for 20 min at 4° C. to precipitate protein. Extracts were then dried in an evaporator (Genevac EZ-2 Elite) overnight. For gas chromatography-mass spectrometry (GC-MS), metabolites were resuspended by addition of 50 μl of methoxyamine hydrochloride (40 mg/ml in pyridine) and incubated at 30° C. for 90 min with agitation (1400 rpm). Metabolites were further derivatized by addition of 80 μl of N-methyl-N-(trimethylsilyl) trifluoroacetamide (MSTFA) plus 1% 2,2,2-trifluoro-N-methyl-N-(trimethylsilyl)-acetamide, chlorotrimethylsilane (TCMS; Thermo Scientific) and 70 μl of ethyl acetate (Sigma) with incubation at 37° C. for 30 min. Samples were diluted 1:2 with 200 μl of ethyl acetate, then analyzed using an Agilent 7890B GC coupled to Agilent 5977B mass selective detector. The GC was operated in splitless mode with constant helium carrier gas flow of 1 ml/min and with a HP-5 MS column (Agilent Technologies). The injection volume was 1 μl and the GC oven temperature was ramped from 60° C. to 290° C. over 25 min. Peaks representing compounds of interest were extracted and integrated using MassHunter version 10.0 (Agilent Technologies) and then normalized to both the internal standard (deuterated-2HG) peak area and cell number or protein content determined using the bicinchoninic acid assay (Thermo Fisher Scientific). Steady-state metabolite pool levels were derived by quantifying the following ions: deuterated-2HG m/z 252 (confirmatory ion m/z 354), 2HG m/z 247 (confirmatory ion m/z 349), citrate m/z 375 (confirmatory ion m/z 465), aspartate m/z 232 (confirmatory ion m/z 334), αKG m/z 288 (confirmatory ion m/z 304), succinate m/z 203 (confirmatory ion m/z 247), fumarate m/z 245 (confirmatory ion m/z 312), malate m/z 245 (confirmatory ion m/z 335), pyruvate m/z 189 (confirmatory ion m/z 174), and lactate m/z 191 (confirmatory ion m/z 219). Peaks were manually inspected and verified relative to known spectra for each metabolite.

### Enzyme Assays

NAD⁺, NADH, NADP⁺, NADPH, alpha-ketoglutarate, and (+)-potassium Ds-threo-isocitrate stocks were freshly prepared in water for each experiment. HA- or FLAG-tagged IDH2 enzymes were purified from transfected 293T cells using Pierce Anti-HA Agarose (Thermo Fisher; 26182) or Anti-FLAG M2 Affinity Gel (Sigma; A2220) according to the manufacturer's instructions. Purified enzymes were semi-quantified (Fiji) by denatured gel electrophoresis with Coomassie Blue staining in reference to a defined quantity of recombinant human IDH2 (Abcam; ab198092). Purified enzymes were used at 7.5 or 10 μg/ml as indicated. NADPH was used at 0.33 mM, NADH was used at 0.25 mM and alpha-ketoglutarate was used at 5 mM unless otherwise indicated. The enzyme reaction buffer consisted of HEPES 50 mM, NaCl 150 mM, MgCl2 20 mM, and BSA 0.01%. For NADPH consumption assays, reactions were conducted in UV-transparent 96-well plates (Corning) with reaction volumes of 200 μl. A SpectraMax Plus 384 Microplate Reader (Molecular Devices) was used to monitor the absorbance at 340 nm every 30 seconds throughout the course of the reaction up to 3 hours at 37° C. unless otherwise indicated. For each condition, the mean rate of NADPH or NADH consumption for triplicate control reactions without enzyme was subtracted from the rate of NADPH or NADH consumption for triplicate experimental reactions with enzyme. Reaction velocities were calculated using an extinction coefficient for NADPH/NADH at ε340 of 6,220 M⁻¹ cm⁻¹ and pathlength of 0.56 cm for a 200 μl reaction volume in a standard 96-well plate.

### High-Throughput Chemical Screen

The MicroSource Spectrum Library of 2560 compounds was used to screen for small-molecules that facilitate NADH-dependent 2HG production by IDH2 R140Q. Briefly, enzyme reactions were set up in 384-well plate format with purified enzyme (7.5 μg/ml), NADH (0.5 mM), αKG (10 mM; added last to start reaction), and HEPES buffer. Compounds were screened using a concentration of 10 μM in DMSO. No enzyme groups were used for each condition as negative controls. The cis-mutant IDH2 RQ/IM enzyme was used as a positive control. Reactions were incubated at 37° C., and absorbance at 340 nm was monitored using an EnVision plate reader at 30-minute intervals. Results were analyzed using KNIME. Briefly, concatenated spreadsheets containing absorbance metadata (well number, plate information, absorbance read outs etc.) were merged. For baseline correction, the “no enzyme with drug” values (N1: no enzyme negative control with drug) for each condition were subtracted from the “plus enzyme with drug” values (S: enzyme with drug) for each condition. Then the baseline corrected enzyme values (S−N1) were normalized to the “no enzyme control” values (N2: no enzyme control with DMSO) to calculate robust (median based) percent of “no enzyme control” (POC) and Z-scores. The hit selection criteria were implemented by setting the Z-score cutoff at 4. To confirm hits, enzyme assays were carried out in the same as screening but in a 96-well plate format with 10 μM of each hit, and purified enzyme (7.5 μg/ml), NADH (0.5 mM), and αKG (10 mM). Absorbance was read at 340 nm every 15 sec for 3 hr. After reaction, metabolites were extracted from enzyme reactions to measure 2HG levels using GC/MS as described herein.

### Crystallization and Structure Determination

Several crystal hits for human IDH2 R140Q/I319M (‘RQ/IM’) with NADP+, αKG, and Mg²⁺, and IDH2 R140Q (‘RQ’) with NADP⁺, αKG, Mg²⁺, and clonixin were obtained from the High-Throughput Crystallization Screening Center of the Hauptman-Woodward Medical Research Institute²⁸. These hits were reproduced using the under-oil micro-batch method at 4° C. and 18° C. and subsequently optimized by seeding method. For comparative structural analyses between the two mutant enzymes (RQ and RQ/IM), crystals were grown of ternary complexes with NADP⁺ 3 mM, αKG 20 mM, and Mg²⁺ 3 mM using crystallization condition #1 (Table 3). Crystals of RQ with NADP⁺1 mM, αKG 2 mM, Mg²⁺ 1 mM, and clonixin 5 mM, which contains DMSO 0.7% were produced using crystallization condition #2 (Table 3). Crystals were subsequently transferred into a similar crystallization reagent that were supplemented by ethylene glycol 20% (v/v) and flash-frozen in liquid nitrogen. A native dataset was collected on a crystal of each complex at the NE-CAT 24-ID-C and 24-ID-E beam lines of Advanced Photon Source in Lemont, IL. Images were processed and scaled using XDS²⁹. The structure of the binary complex of RQ/IM with NADP⁺ was initially determined by molecular replacement method using MOLREP³⁰ and the crystal structure of IDH2 with NADP⁺, αKG, and Ca²⁺ (PDB id: 5195) as the initial search model. The geometry of each crystal structure was subsequently fixed and small molecules (cofactors, substrates, product, clonixin) were modeled by XtalView³¹ and COOT³² then refined by Phenix³³. Crystallographic statistics are shown in Table 2.

### Statistical Analysis

Statistical analyses were performed using Microsoft Excel and GraphPad Prism 9. Significance was determined by two-tailed Student's t-test comparing the indicated condition to the corresponding controls. The number of replicates is indicated as open circles in figures. For quantitative measurements, n represents the number of biological replicates and is provided in the figure legends. Results were independently replicated at least three times. P values throughout are defined as follows: ns (not significant; P>0.05), *P<0.05, **P<0.01, ***P<0.001, ****P<0.0001.

### Data Availability

Crystal structures were deposited into Protein Data Bank with the following IDs: 8FXK for IDH2 R140Q, 8FXL for IDH2 R140Q with clonixin, and 8FXM for IDH2 R140Q/I319M.

### REFERENCES CITED IN THIS EXAMPLE

1. Losman, J. A. & Kaelin, W. G., Jr. What a difference a hydroxyl makes: mutant IDH, (R)-2-hydroxyglutarate, and cancer. Genes & Development 27, 836-852, doi:10.1101/gad.217406.113 (2013).
2. Ye, D., Guan, K. L. & Xiong, Y. Metabolism, Activity, and Targeting of D- and L-2-Hydroxyglutarates. Trends Cancer 4, 151-165, doi:10.1016/j.trecan.2017.12.005 (2018).
3. Yen, K. et al. AG-221, a First-in-Class Therapy Targeting Acute Myeloid Leukemia Harboring Oncogenic IDH2 Mutations. Cancer Discovery 7, 478-493, doi:10.1158/2159-8290.CD-16-1034 (2017).
4. Wang, F. et al. Targeted inhibition of mutant IDH2 in leukemia cells induces cellular differentiation. Science 340, 622-626, doi:10.1126/science.1234769 (2013).
5. Rohle, D. et al. An inhibitor of mutant IDH1 delays growth and promotes differentiation of glioma cells. Science 340, 626-630, doi:10. 1126/science.1236062 (2013).
6. Stein, E. M. et al. Enasidenib in mutant IDH2 relapsed or refractory acute myeloid leukemia. Blood 130, 722-731, doi:10.1182/blood-2017-04-779405 (2017).
7. DiNardo, C. D. et al. Durable Remissions with Ivosidenib in IDH1-Mutated Relapsed or Refractory AML. The New England Journal of Medicine 378, 2386-2398, doi:10.1056/NEJMoa1716984 (2018).
8. Quek, L. et al. Clonal heterogeneity of acute myeloid leukemia treated with the IDH2 inhibitor enasidenib. Nature Medicine 24, 1167-1177, doi: 10.1038/s41591-018-0115-6 (2018).
9. Wang, F. et al. Leukemia stemness and co-occurring mutations drive resistance to IDH inhibitors in acute myeloid leukemia. Nat Commun 12, 2607, doi:10.1038/s41467-021-22874-x (2021).
10. Harding, J. J. et al. Isoform Switching as a Mechanism of Acquired Resistance to Mutant Isocitrate Dehydrogenase Inhibition. Cancer Discovery 8, 1540-1547, doi:10.1158/2159-8290.CD-18-0877 (2018).
11. Intlekofer, A. M. et al. Acquired resistance to IDH inhibition through trans or cis dimer-interface mutations. Nature 559, 125-129, doi:10.1038/s41586-018-0251-7 (2018).
12. Choe, S. et al. Molecular mechanisms mediating relapse following ivosidenib monotherapy in IDH1-mutant relapsed or refractory AML. Blood Adv 4, 1894-1905, doi:10.1182/bloodadvances.2020001503 (2020).
13. Lyu, J. et al. Disabling Uncompetitive Inhibition of Oncogenic IDH Mutations Drives Acquired Resistance. Cancer Discov 13, 170-193, doi:10.1158/2159-8290.CD-21-1661 (2023).
14. Gorelick, A. N. et al. Phase and context shape the function of composite oncogenic mutations. Nature 582, 100-103, doi:10.1038/s41586-020-2315-8 (2020).
15. Valley, C. C. et al. The methionine-aromatic motif plays a unique role in stabilizing protein structure. The Journal of Biological Chemistry 287, 34979-34991, doi:10.1074/jbc.M112.374504 (2012).
16. Vernon, R. M. et al. Pi-Pi contacts are an overlooked protein feature relevant to phase separation. Elife 7, doi:10.7554/eLife.31486 (2018).
17. Brunger, A. T. Version 1.2 of the Crystallography and NMR system. Nat Protoc 2, 2728-2733, doi:10.1038/nprot.2007.406 (2007).
18. Reinbold, R. et al. Resistance to the isocitrate dehydrogenase 1 mutant inhibitor ivosidenib can be overcome by alternative dimer-interface binding inhibitors. Nat Commun 13, 4785, doi:10.1038/s41467-022-32436-4 (2022).
19. Zorn, J. A. & Wells, J. A. Turning enzymes ON with small molecules. Nature Chemical Biology 6, 179-188, doi:10.1038/nchembio.318 (2010).
20. Finch, J. S. & DeKornfeld, T. J. Clonixin: a clinical evaluation of a new oral analgesic. J Clin Pharmacol New Drugs 11, 371-377 (1971).
21. Shih, A. H. et al. Combination Targeted Therapy to Disrupt Aberrant Oncogenic Signaling and Reverse Epigenetic Dysfunction in IDH2- and TET2-Mutant Acute Myeloid Leukemia. Cancer Discovery 7, 494-505, doi:10.1158/2159-8290.CD-16-1049 (2017).
22. Chan, S. M. et al. Isocitrate dehydrogenase 1 and 2 mutations induce BCL-2 dependence in acute myeloid leukemia. Nature Medicine 21, 178-184, doi:10.1038/nm.3788 (2015).
23. Fu, X. et al. 2-Hydroxyglutarate Inhibits ATP Synthase and mTOR Signaling. Cell Metabolism 22, 508-515, doi:10.1016/j.cmet.2015.06.009 (2015).
24. McBrayer, S. K. et al. Transaminase Inhibition by 2-Hydroxyglutarate Impairs Glutamate Biosynthesis and Redox Homeostasis in Glioma. Cell 175, 101-116 e125, doi:10.1016/j.cell.2018.08.038 (2018).
25. Hollinshead, K. E. R. et al. Oncogenic IDH1 Mutations Promote Enhanced Proline Synthesis through PYCR1 to Support the Maintenance of Mitochondrial Redox Homeostasis. Cell Reports 22, 3107-3114, doi:10.1016/j.celrep.2018.02.084 (2018).
26. Thomas, D. et al. Dysregulated lipid synthesis by oncogenic IDH1 mutation is a targetable synthetic lethal vulnerability. Cancer Discovery, doi:10.1158/2159-8290.CD-21-0218 (2022).
27. Amatangelo, M. D. et al. Enasidenib induces acute myeloid leukemia cell differentiation to promote clinical response. Blood 130, 732-741, doi:10.1182/blood-2017-04-779447 (2017).
28. Luft, J. R. et al. A deliberate approach to screening for initial crystallization conditions of biological macromolecules. J Struct Biol 142, 170-179, doi:10.1016/s1047-8477(03)00048-0 (2003).
29. Kabsch, W. Integration, scaling, space-group assignment and post-refinement. Acta Crystallogr D Biol Crystallogr 66, 133-144, doi:10.1107/S0907444909047374 (2010).
30. Vagin, A. & Teplyakov, A. Molecular replacement with MOLREP. Acta Crystallogr D Biol Crystallogr 66, 22-25, doi:10.1107/S0907444909042589 (2010).
31. McRee, D. E. XtalView/Xfit—A versatile program for manipulating atomic coordinates and electron density. J Struct Biol 125, 156-165, doi:10.1006/jsbi. 1999.4094 (1999).
32. Emsley, P., Lohkamp, B., Scott, W. G. & Cowtan, K. Features and development of Coot. Acta Crystallogr D Biol Crystallogr 66, 486-501, doi:10.1107/S0907444910007493 (2010).
33. Adams, P. D. et al. PHENIX: a comprehensive Python-based system for macromolecular structure solution. Acta Crystallogr D Biol Crystallogr 66, 213-221, doi:10.1107/S0907444909052925 (2010).

### Example 2

### A Strategy to Target Cancer Metabolism

Mutations in isocitrate dehydrogenase (IDH) enzymes are hallmarks of a variety of deadly cancers, including acute myeloid leukemia (AML), glioma, cholangiocarcinoma, chondrosarcoma, and T cell lymphoma. Mutant IDH enzymes drive cancer through an unusual mechanism—they produce a metabolite called 2-hydroxyglutarate (2HG) that poisons gene expression machinery and locks malignant cells in a stem cell-like state. Drugs that inhibit mutant IDH enzymes induce durable clinical responses in approximately 40% of patients with treatment-refractory IDH-mutant acute myeloid leukemia (AML), leading to their recent FDA approvals for this indication. However, despite inhibition of 2HG production, over half of patients do not respond to IDH inhibitors. Even for patients who initially respond to IDH inhibitors, most eventually acquire resistance to the drugs. Emerging evidence indicates that acquisition of specific co-occurring mutations (e.g. activating mutations in the RAS/MAPK pathway) during tumor evolution results in a loss of dependence on 2HG. Therefore, we need new treatment approaches that target IDH mutations in different ways beyond simple inhibition of the enzyme. Based on mechanistic studies of an unusual drug resistance mutation identified in patients with acquired resistance to IDH inhibitors (Intlekofer, Shih et al, Nature 2018), we uncovered a new strategy to target cancer-associated mutations in isocitrate dehydrogenase 2 (IDH2). Using high-throughput chemical screens, we identified small molecules which can hyperactivate and deregulate mutant IDH2, resulting in selective toxicity towards IDH2-mutant leukemia cells. This is a new and unexpected therapeutic approach, which transforms the IDH2 mutation into a lethal metabolic liability and overcomes limitations of existing strategies that inhibit mutant IDH2 enzymatic function. We can optimize these small molecules and validate their function in mouse models of IDH2-mutant leukemia and other tumor types. These studies will also provide a framework to develop small-molecule activators of other metabolic enzymes important in different cancer contexts. By harnessing this new approach of enzyme hyperactivation, we can produce more effective drugs and improve outcomes for a wide array of cancers driven by deregulated cellular metabolism.

### Example 3

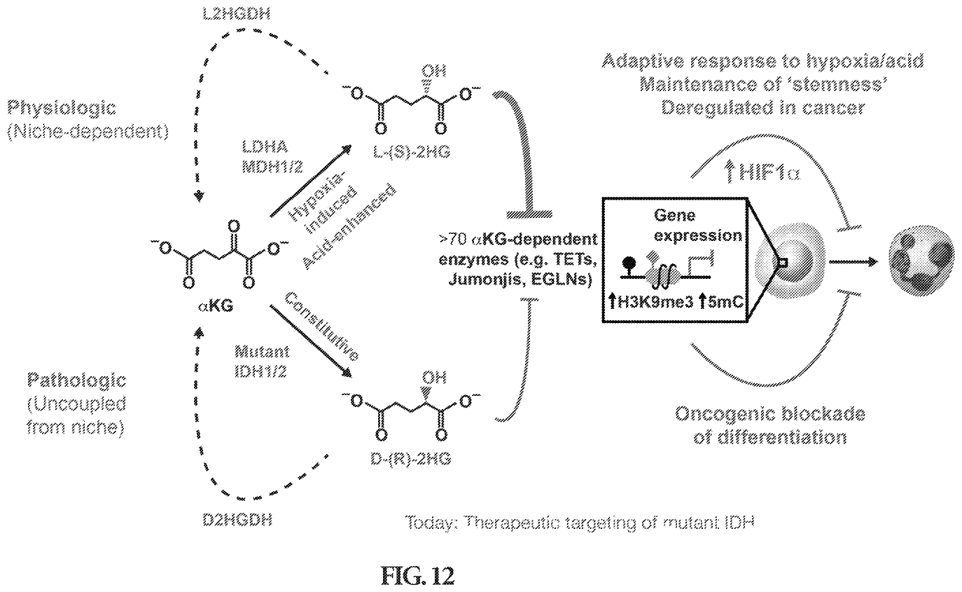

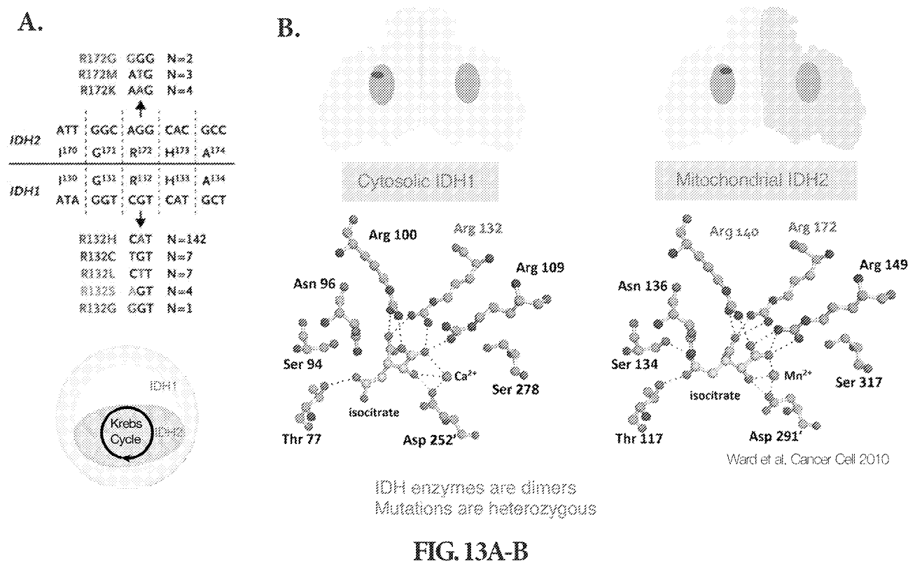

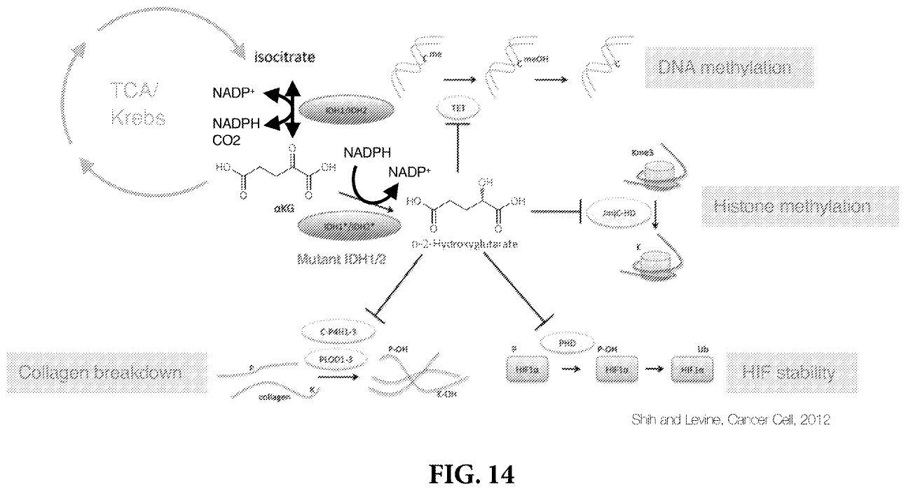

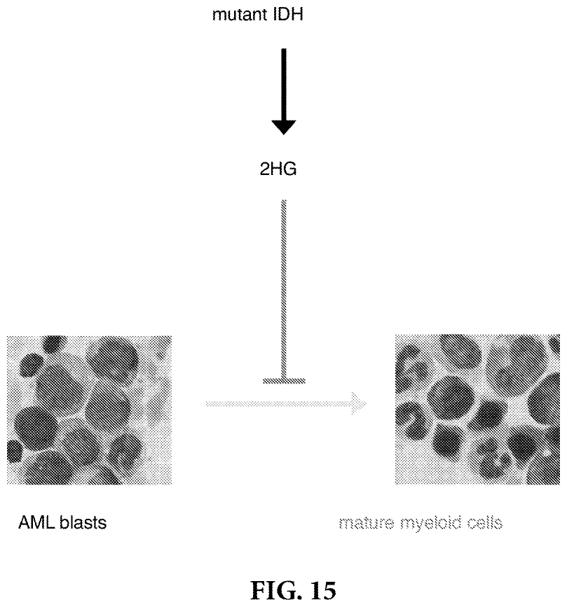

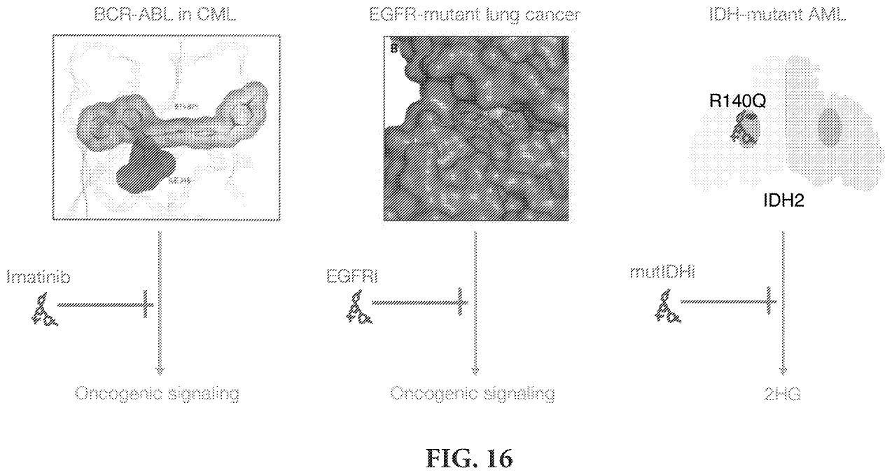

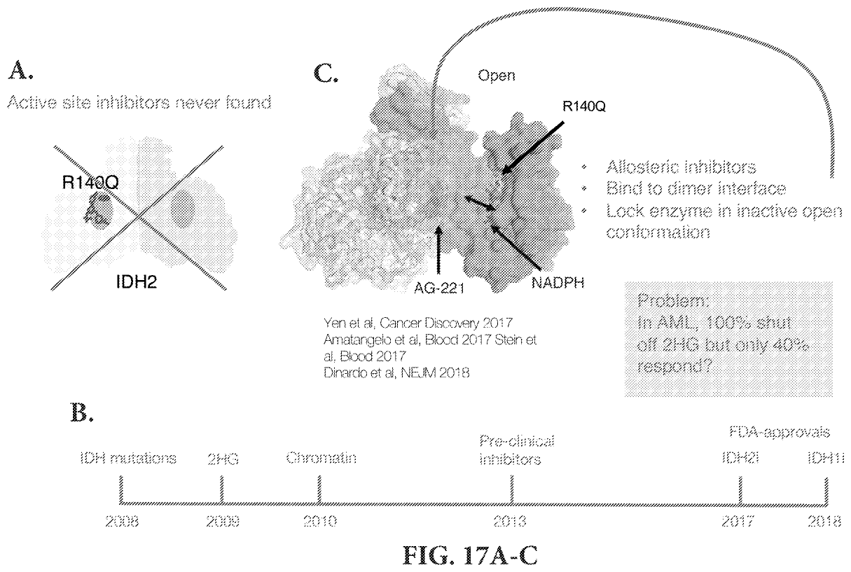

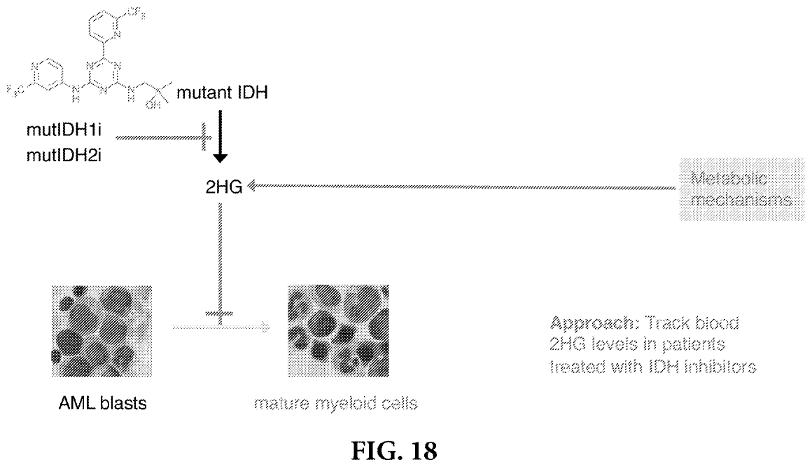

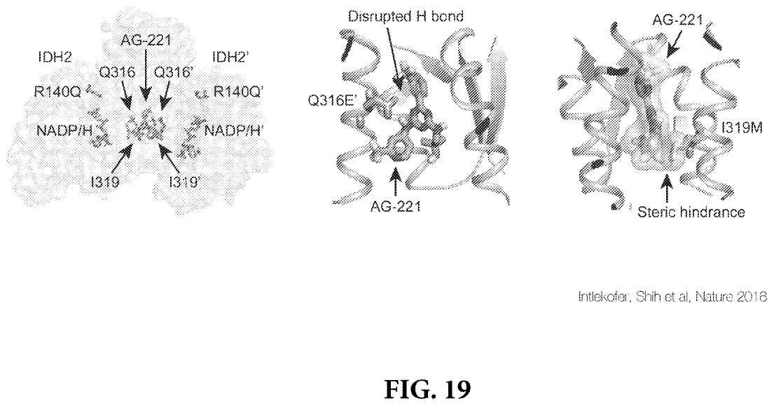

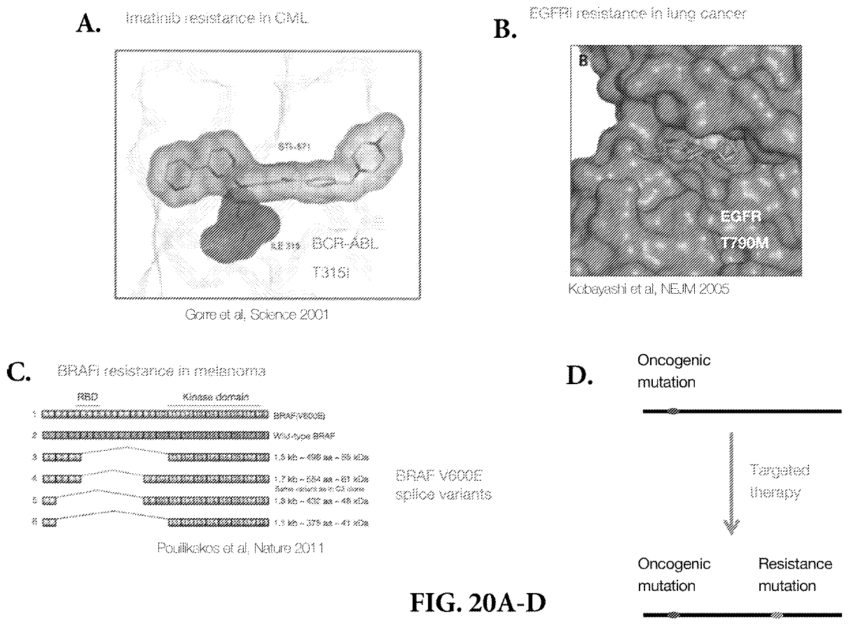

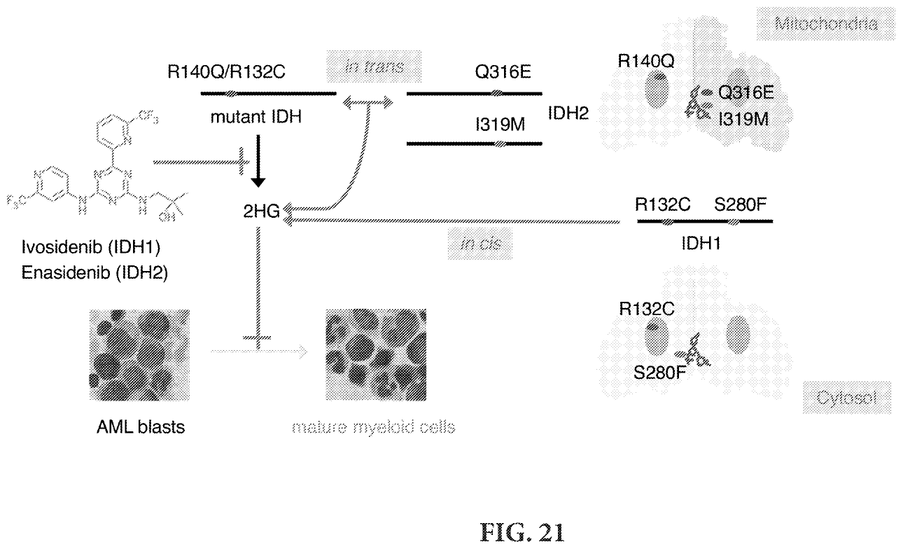

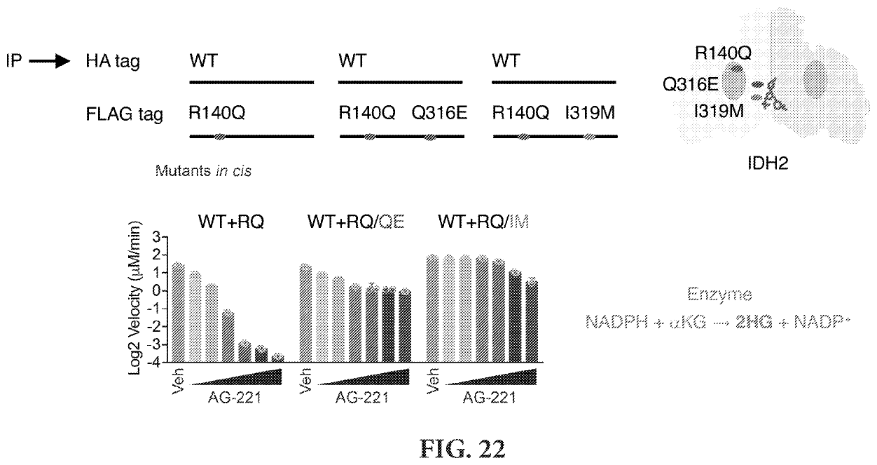

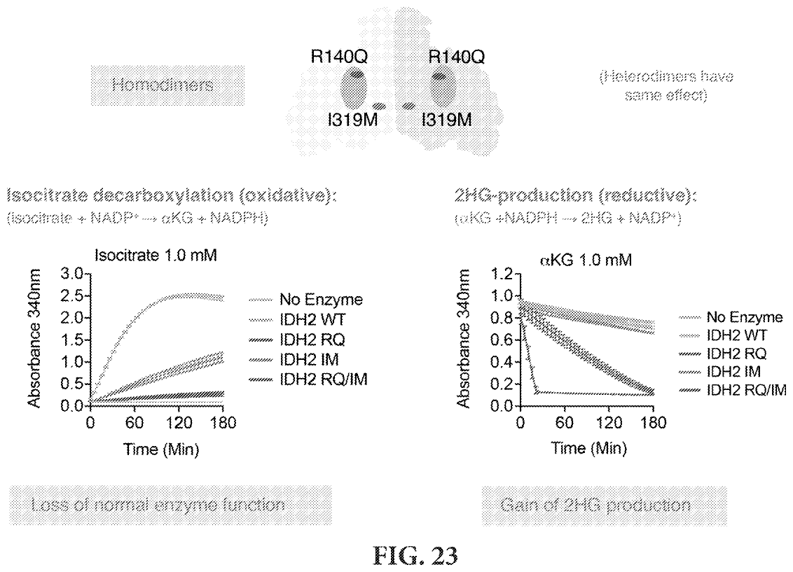

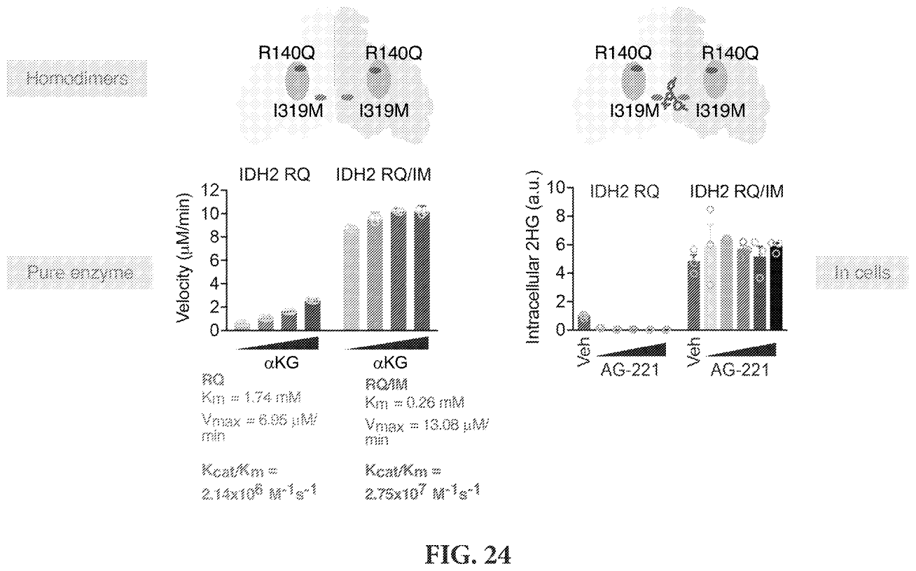

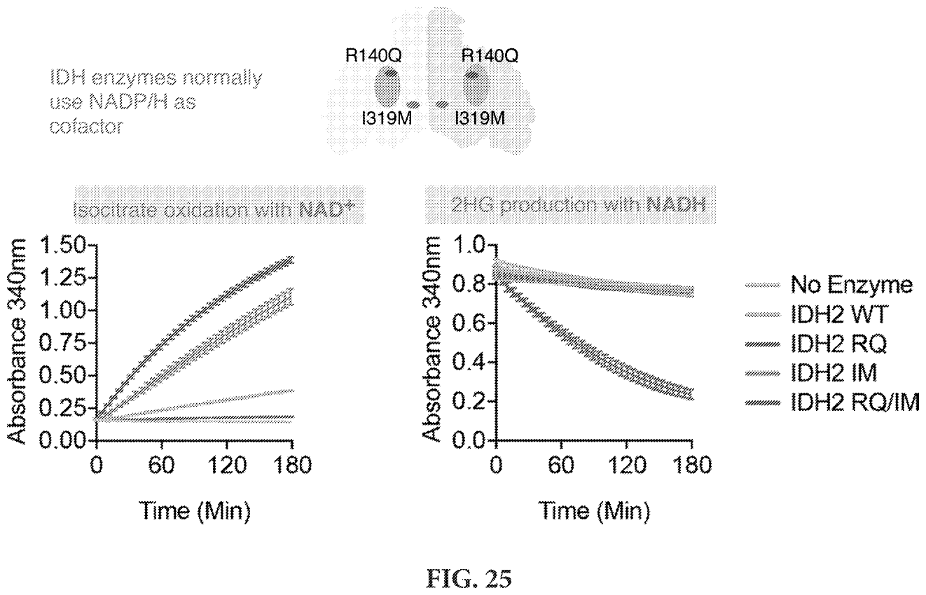

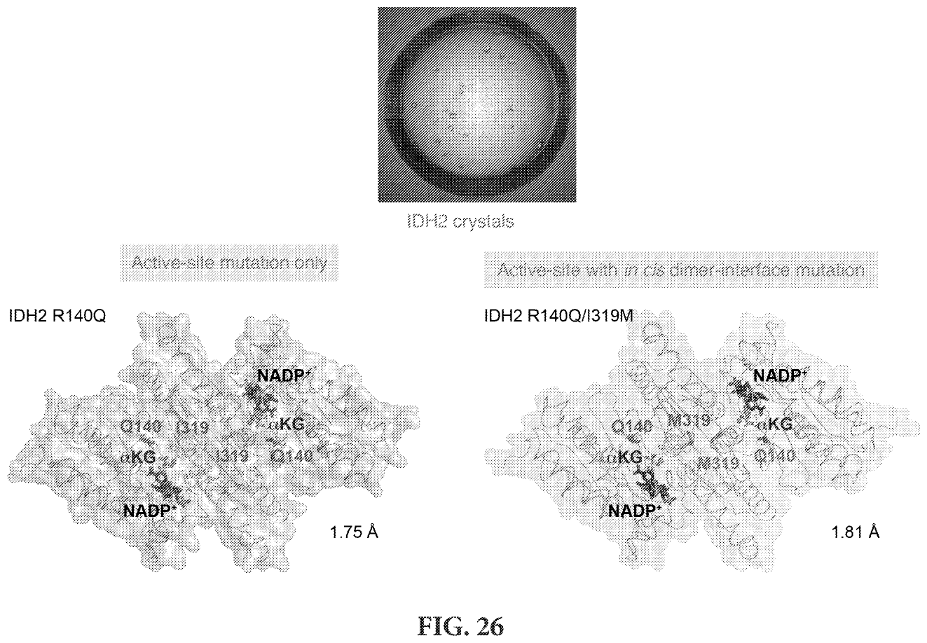

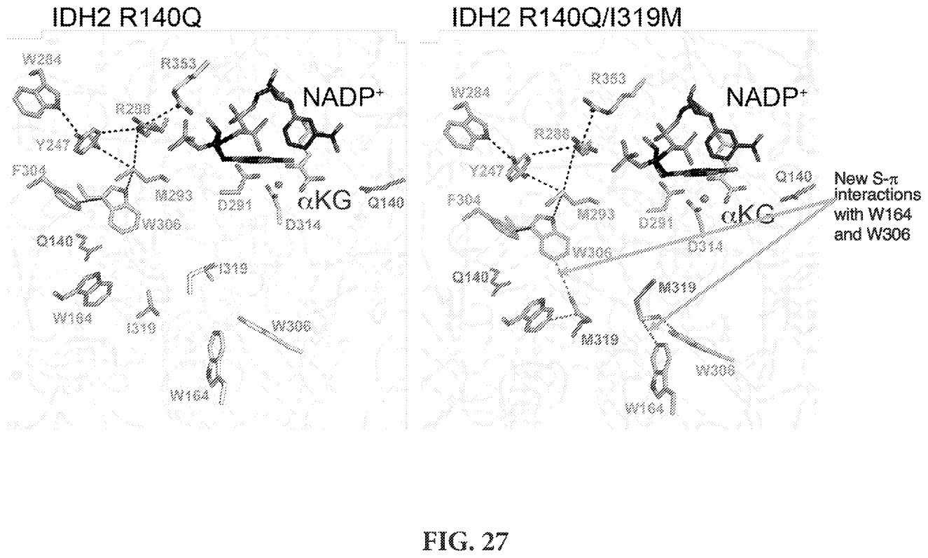

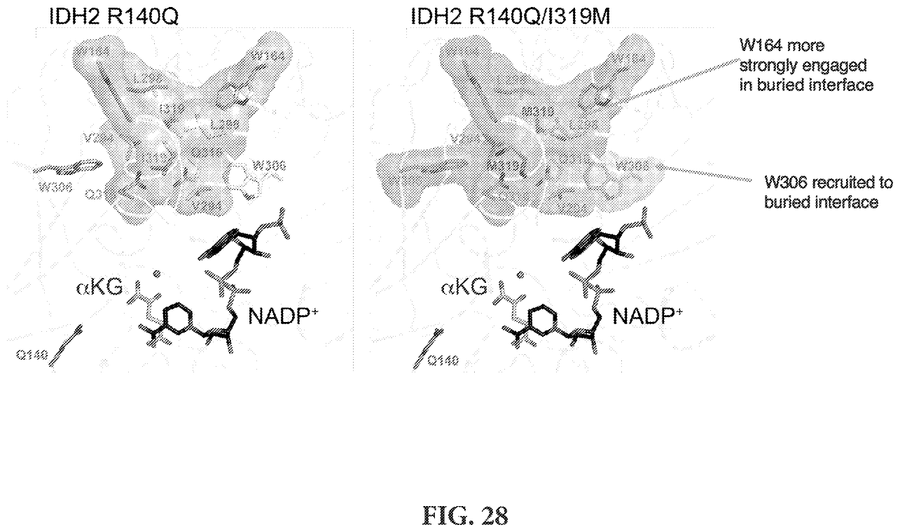

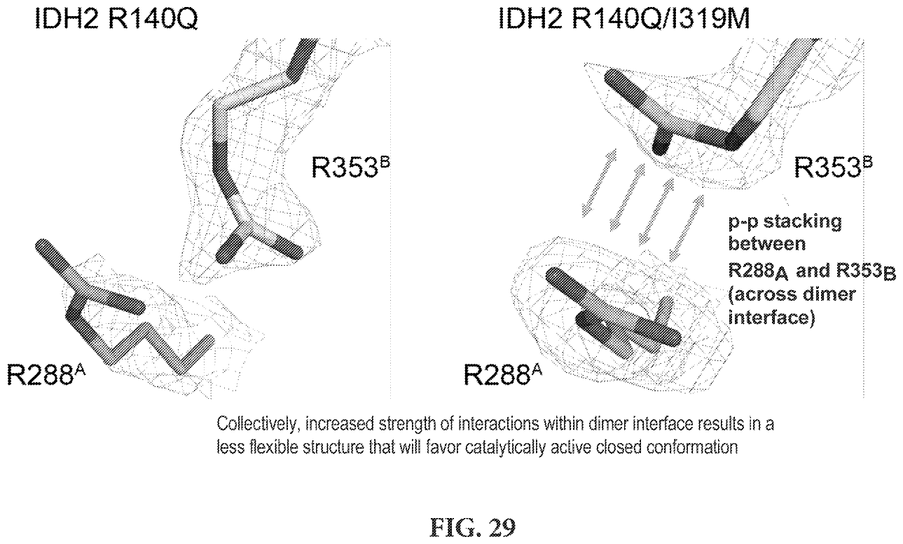

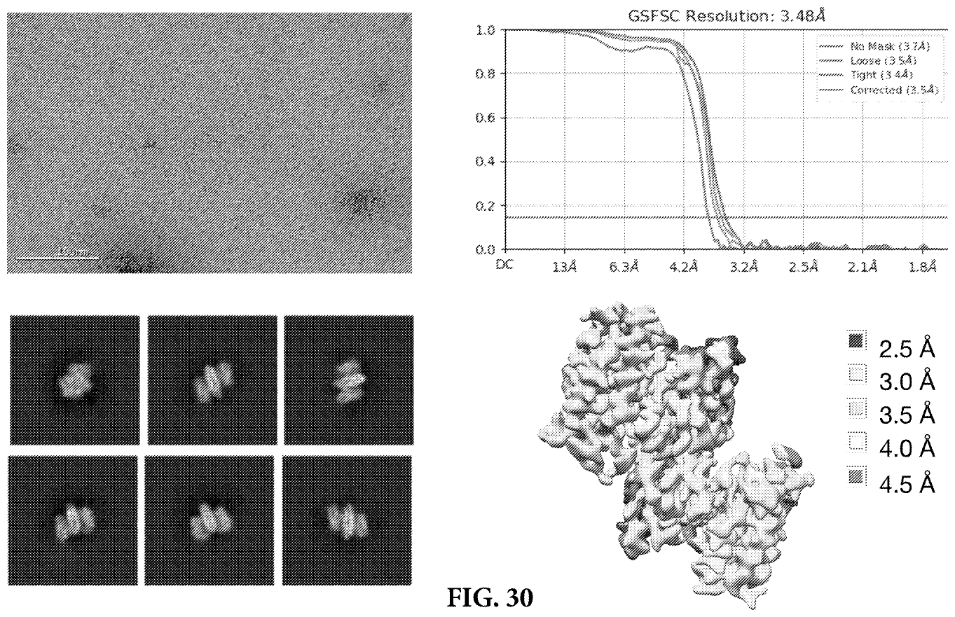

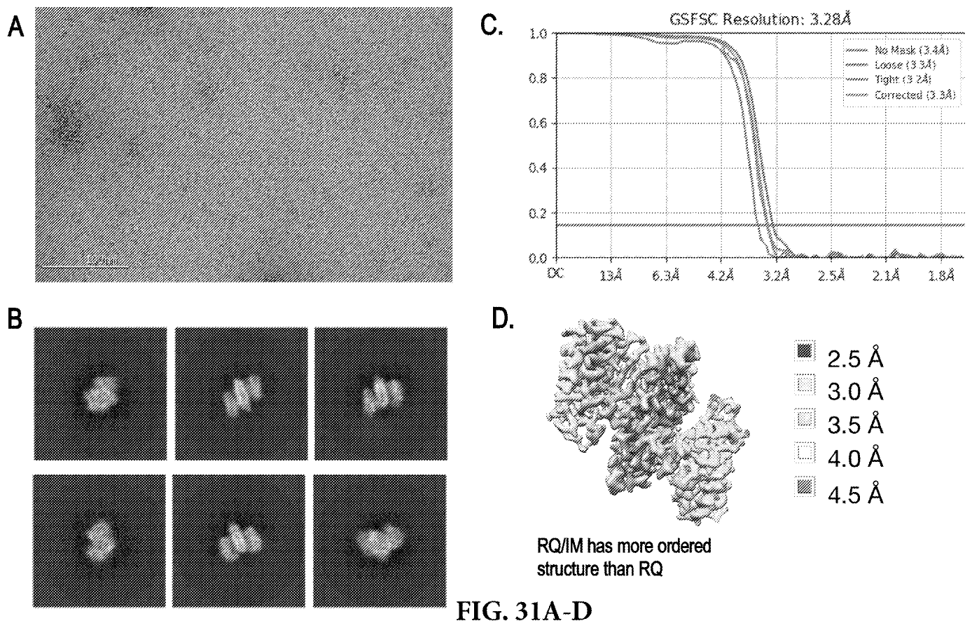

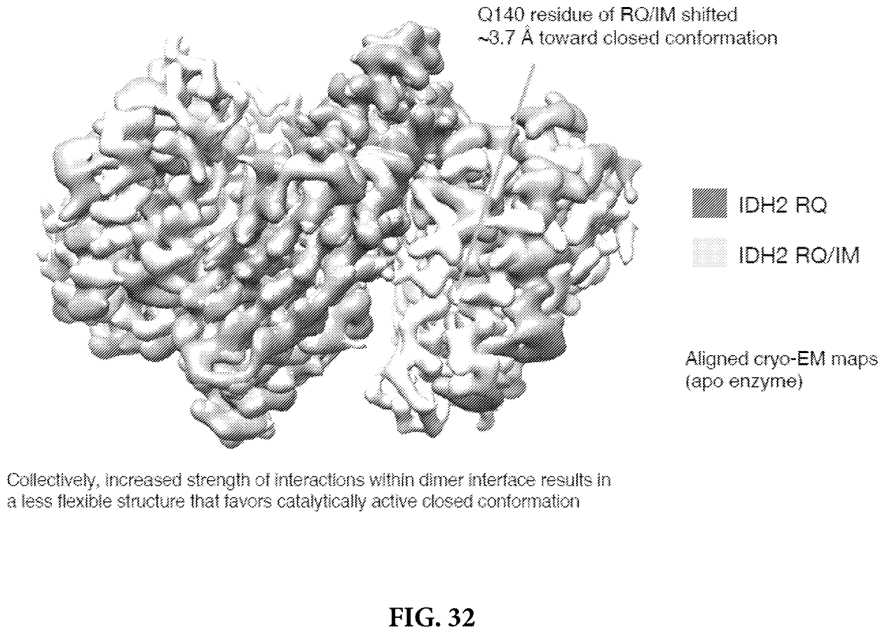

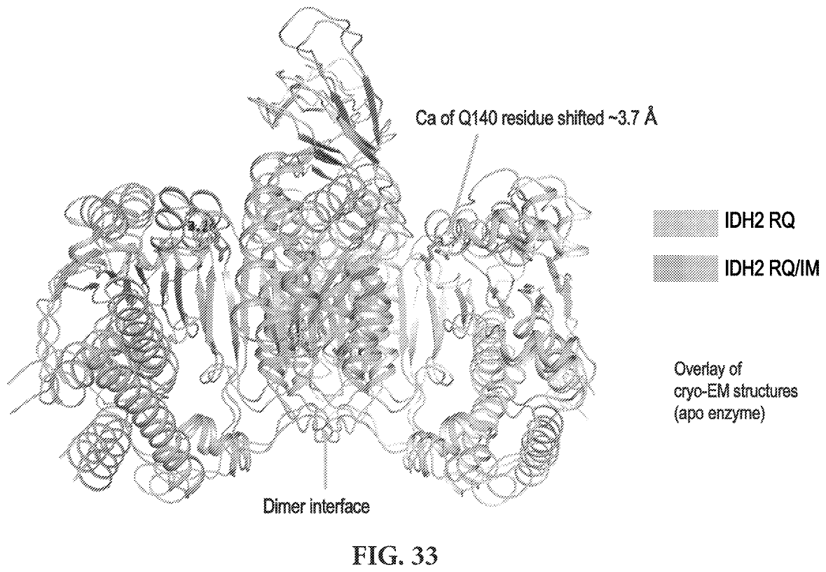

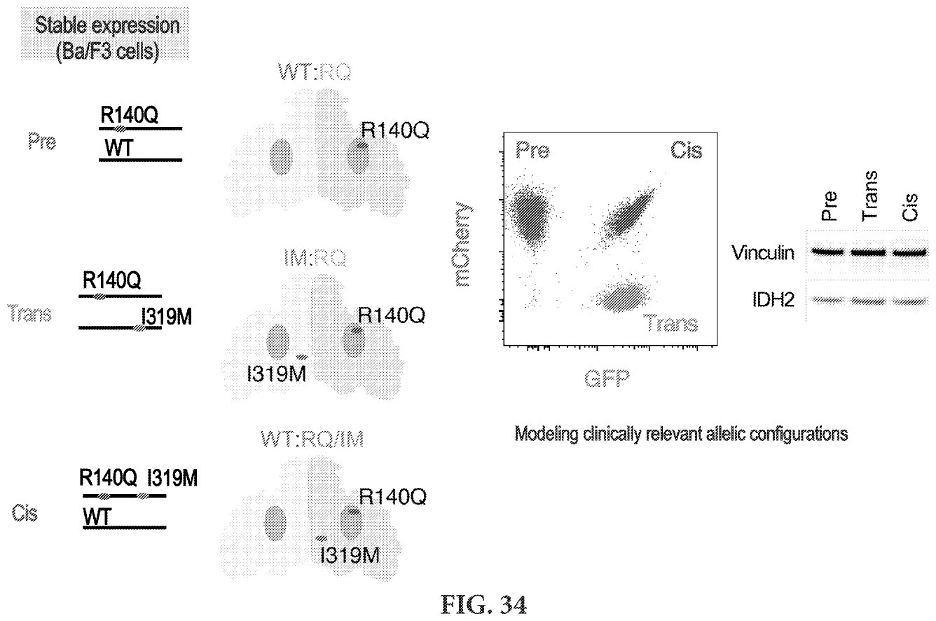

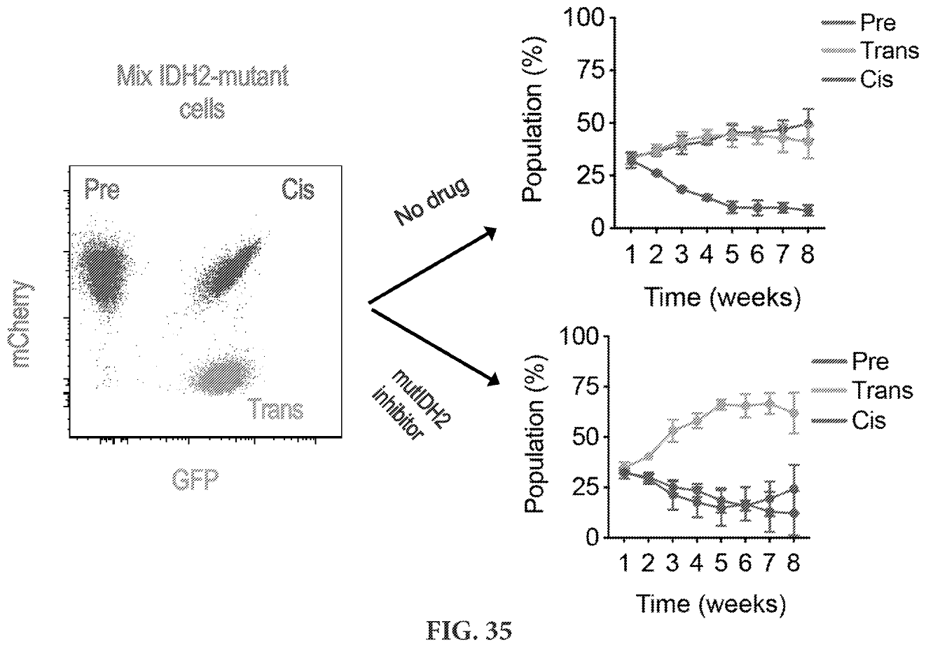

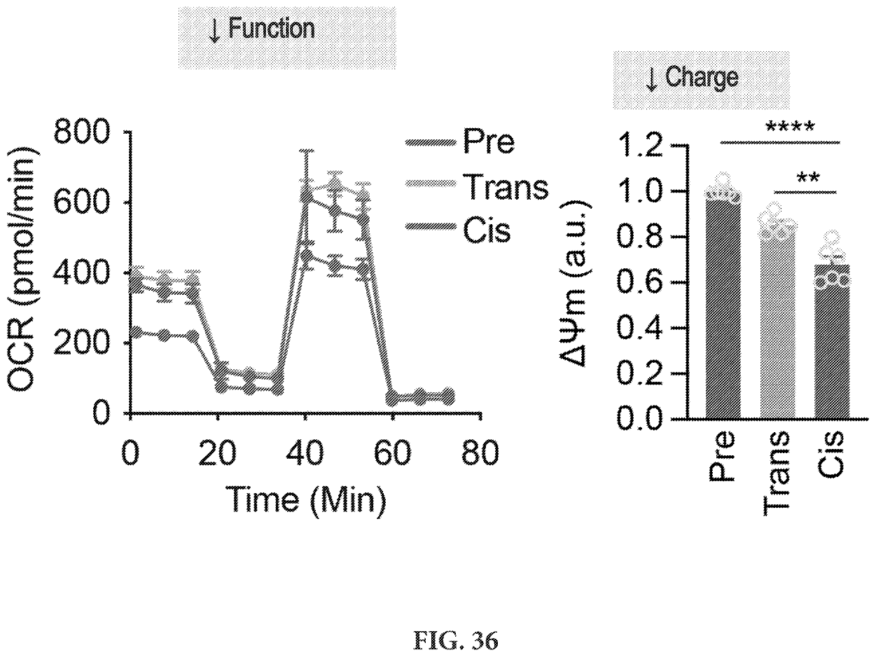

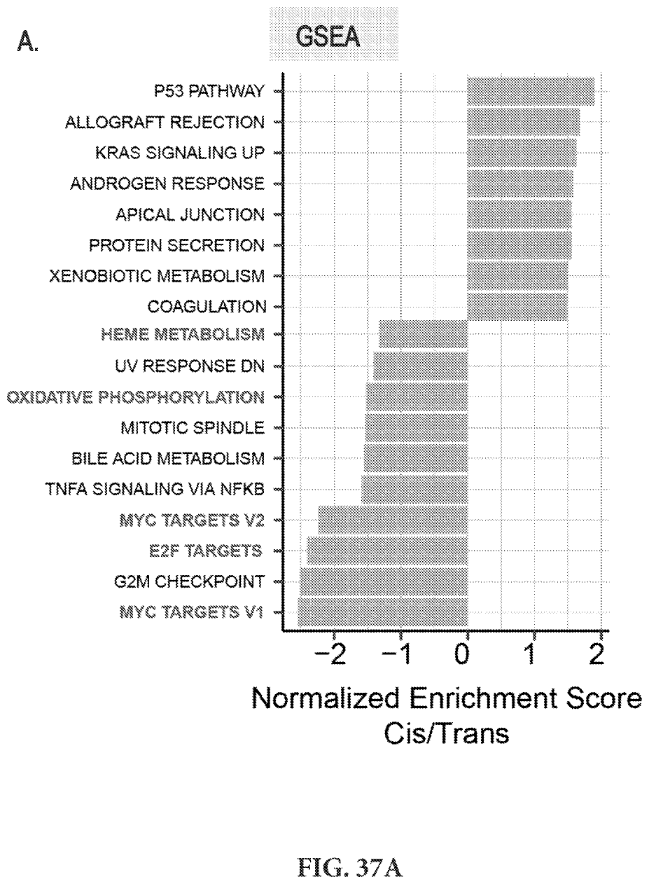

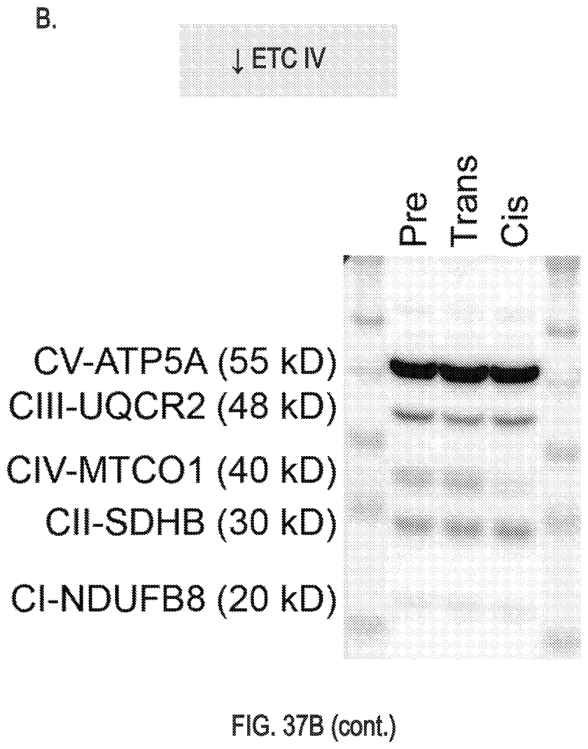

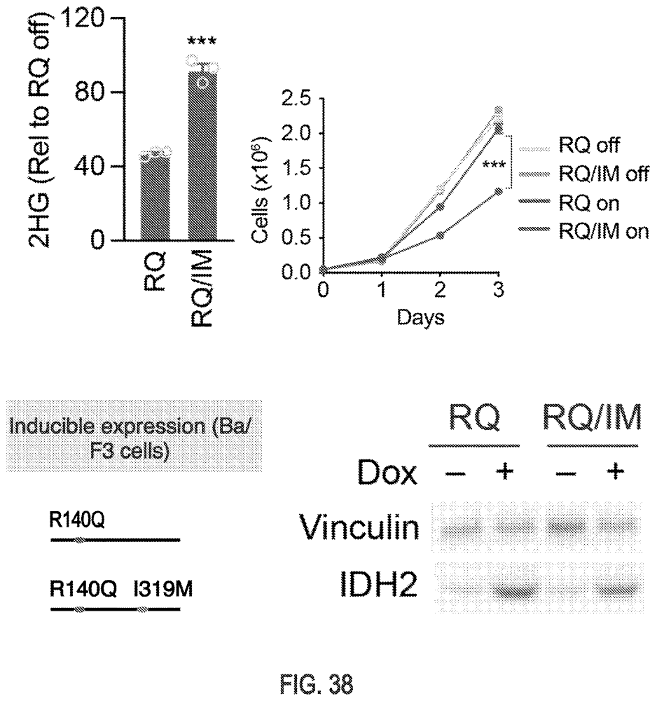

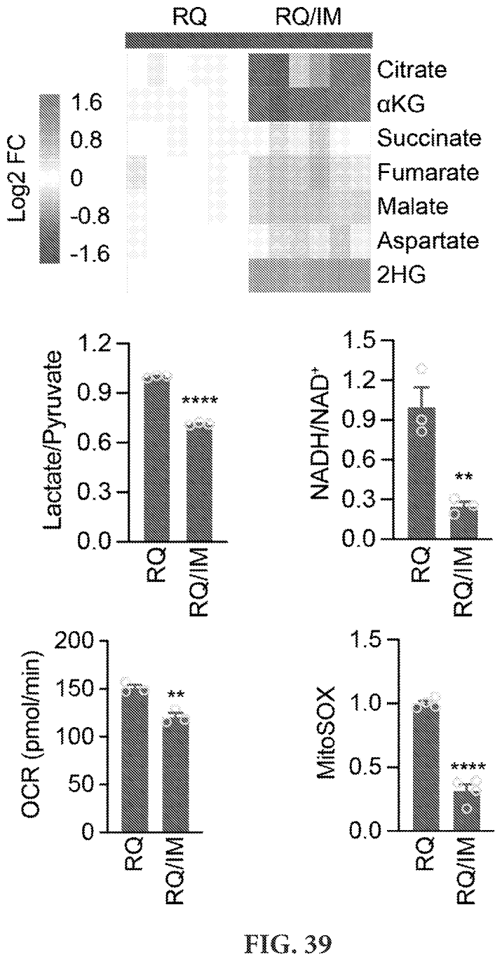

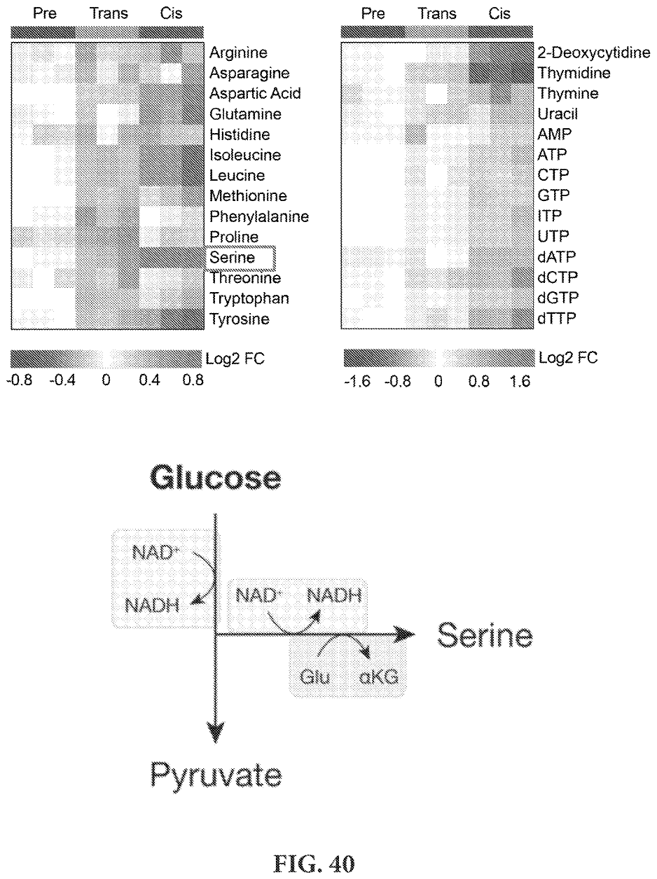

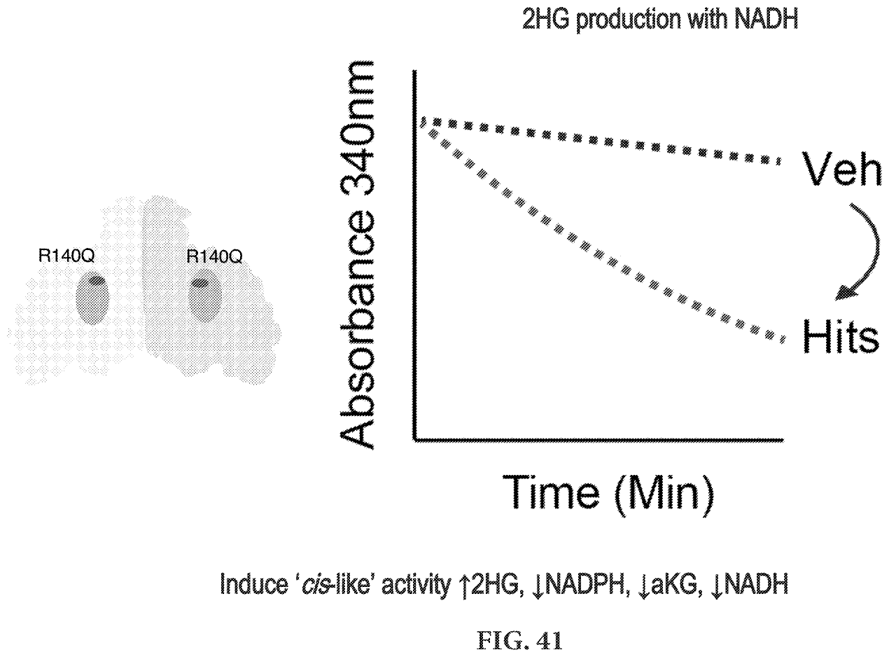

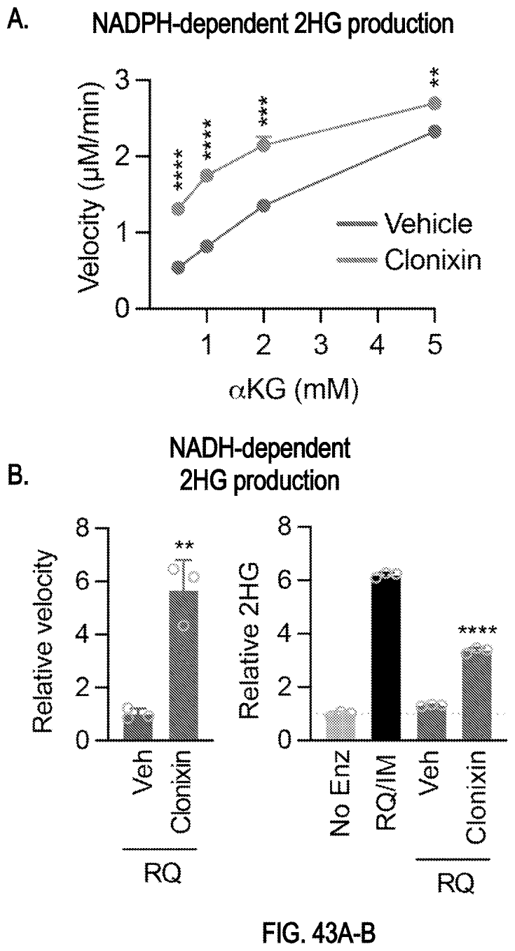

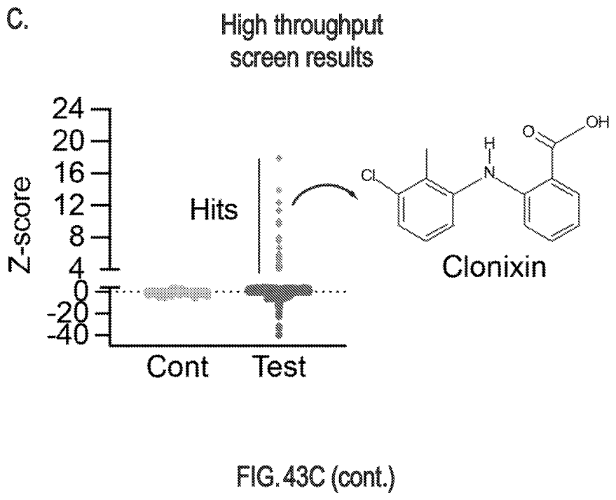

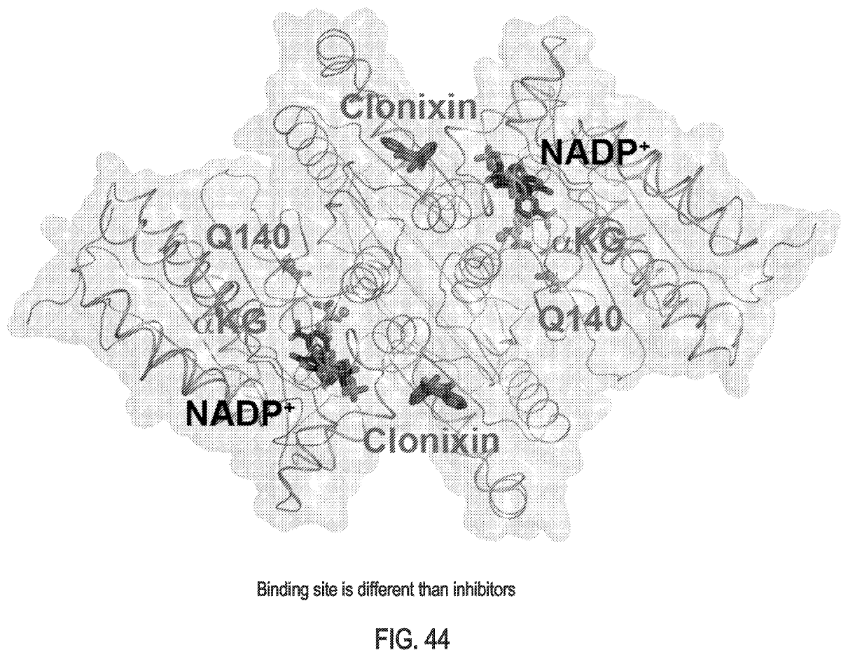

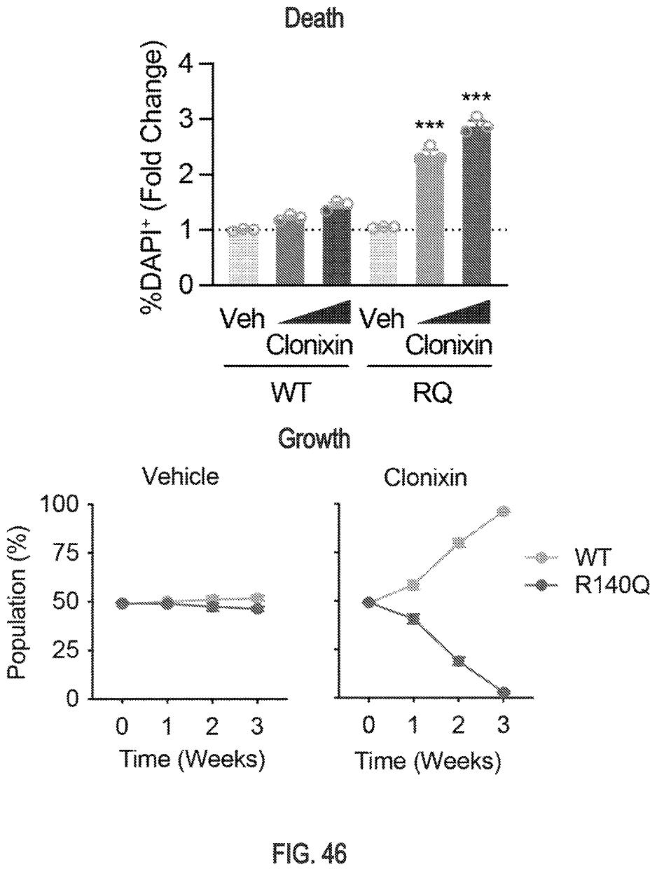

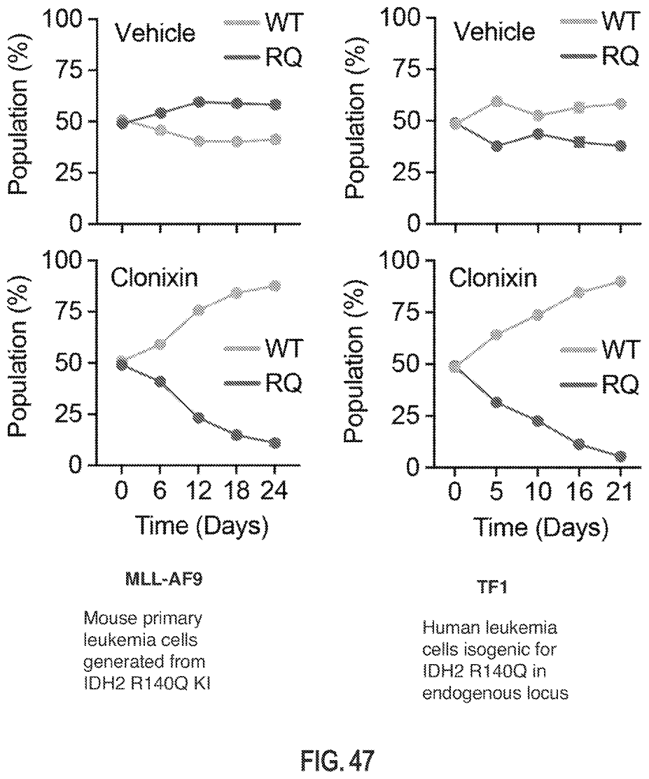

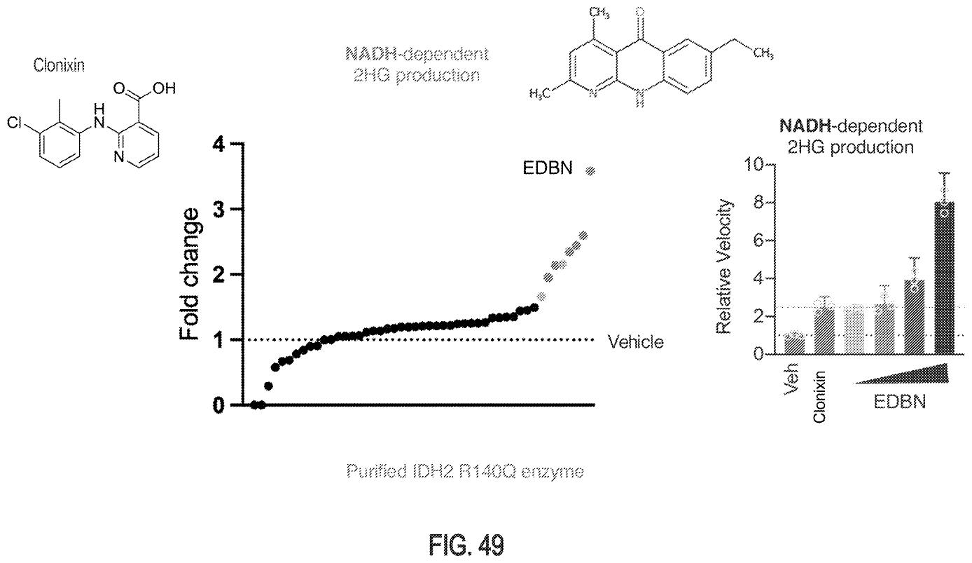

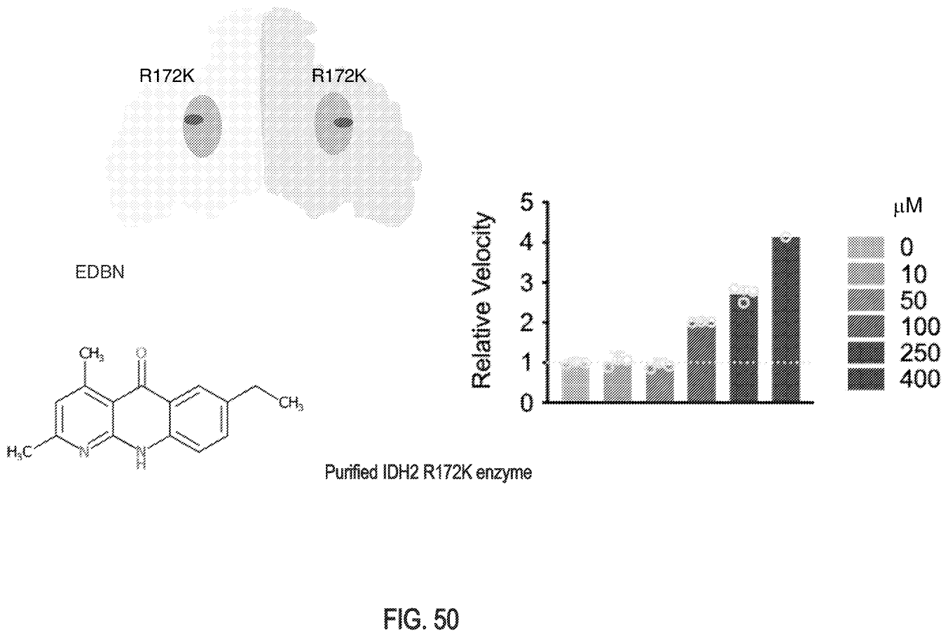

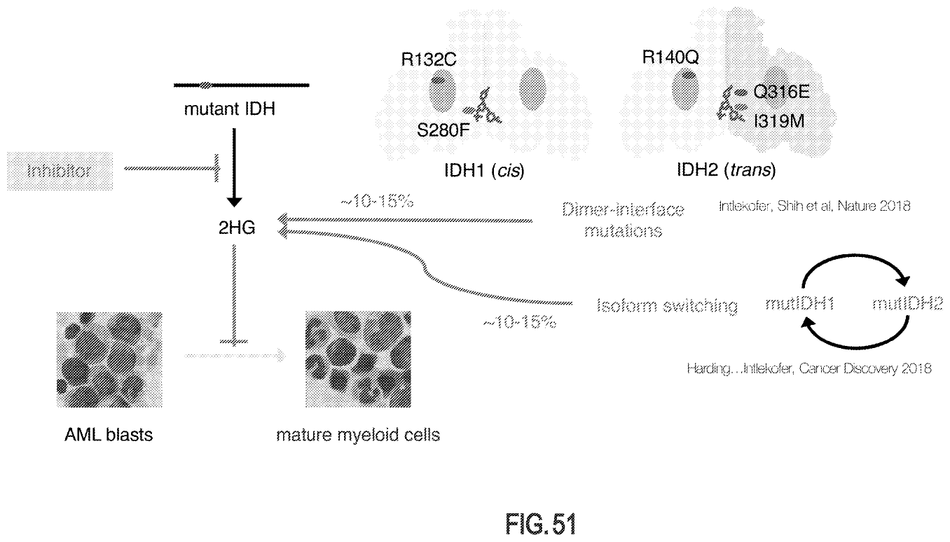

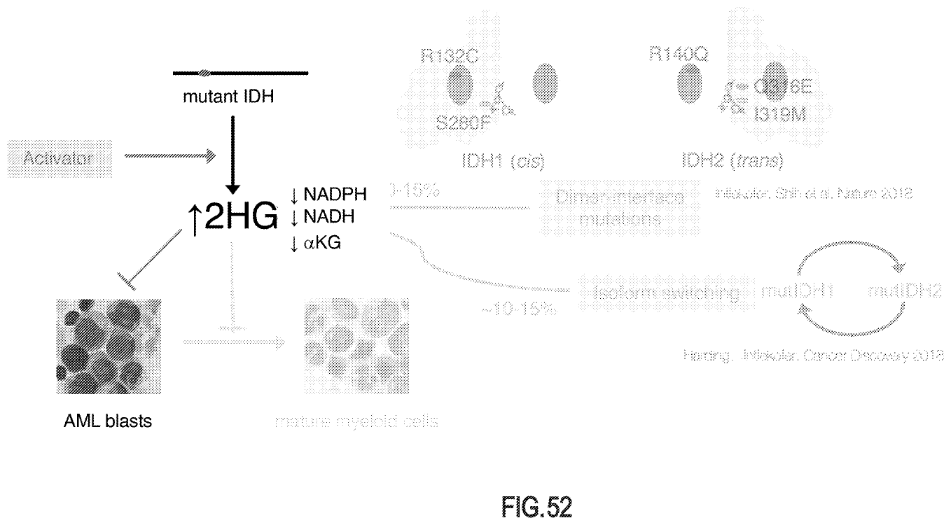

Enzyme hyperactivation unleashes toxicity from isocitrate dehydrogenase mutations
Cancer associated IDH mutations cluster in the enzyme active site (FIG. 13).
IDH mutants produce ‘oncometabolite’ 2-hydroxyglutarate (2HG) (FIG. 14).
2HG locks IDH-mutant cells in a stem cell-like state (FIG. 15).
Obvious solution: Develop a drug that inhibits the mutant IDH enzyme (FIG. 16).
An unexpected type of mutant IDH inhibitor developed (FIG. 17).
Can IDH-mutant tumors ‘figure out’ ways to resist the inhibitors and restore 2HG (FIG. 18)?
Resistance mutations can occur at IDH dimer interface where drug binds (FIG. 19). Secondary mutations occur in key residues for drug binding. Q316-hydrogen bonds from amino and carbonyl sidechain and carbonyl backbone. I319-van der Waals interactions.
Classic drug-resistance mutations in oncogenes occur in cis (on same copy of gene) (FIG. 20).
Dimer-interface mutations confer resistance to mutant IDH inhibitors (FIG. 21).
IDH2 dimer-interface mutations in cis can biochemically confer drug resistance (FIG. 22).
Dimer-interface mutation in cis selectively alters IDH2 enzyme function (FIG. 23).
The in cis mutation turns IDH2 into a super 2HG producer (FIG. 24).
Dimer-interface mutation in cis ‘confuses’ IDH2 and allows it to use the wrong cofactor (FIG. 25).
Cis mutation I319M ‘glues’ the IDH2 dimer more tightly together (FIGS. 27-29).
Cis-mutant IDH2 adopts a closed conformation compared to active-site mutant (FIGS. 32-33).
Without wishing to be bound by theory, the cis-mutant enzyme has detrimental effects on cell fitness (FIG. 34).
Dimer-interface mutation in cis impairs leukemia cell growth (FIG. 35-37).
IDH2 dimer-interface mutation in cis inhibits cell proliferation (FIG. 38).
IDH2 dimer-interface mutation in cis causes metabolic dysfunction (FIGS. 38-39).
Can we identify small-molecule activators of mutant IDH2 (FIG. 41).
Screen results: Spectrum MicroSource Library of 2560 compounds (FIG. 42).
Identification of small molecules that activate mutant IDH2 (FIGS. 43-44).
Structural basis for mutant IDH2 activation (FIG. 45).
Selective toxicity for IDH2-mutant leukemia cells (FIGS. 46-47).
SAR-guided lead optimization (ongoing) (FIGS. 48-49).
EDBN also works on IDH2 R172X mutations (FIG. 50).
Structure-guided lead optimization (ongoing).
Therapeutic targeting of mutant IDH enzymes (FIG. 51).
Hyperactivation instead of inhibition for mutant IDH enzymes (FIG. 52).

### EQUIVALENTS

Those skilled in the art will recognize, or be able to ascertain, using no more than routine experimentation, numerous equivalents to the specific substances and procedures described herein. Such equivalents are considered to be within the scope of this invention, and are covered by the following claims.

What is claimed:

1. A method for treating a cancer in a subject, the method comprising hyperactivating an oncogenic activity of an enzyme in cancer cells.

2. The method of claim 1, wherein the oncogenic activity of the enzyme is not inhibited.

3. The method of claim 1, wherein the enzyme comprises a mutated endogenous enzyme.

4. The method of claim 3, wherein the endogenous enzyme does not have oncogenic activity.

5. The method of claim 3, wherein the mutated enzyme acquired oncogenic activity.

6. The method of claim 1, wherein the enzyme that has the oncogenic activity comprises a mutant isocitrate dehydrogenase (mutIDH).

7. The method of claim 6, wherein the mutIDH catalyzes a reaction of α-ketoglutarate (AKG) to 2-hydroxyglutarate (2HG).

8. The method of claim 6, wherein hyperactivating the oncogenic activity of the mutIDH increases an amount of 2HG in cancer cells.

9. The method of claim 6, wherein hyperactivating the oncogenic activity of the mutIDH depletes AKG, reduces nicotinamide adenine dinucleotide (NADH), reduces nicotinamide adenine dinucleotide phosphate (NADPH) in cancer cells, or a combination thereof.

10. The method of claim 6, wherein hyperactivating the oncogenic activity of the mutIDH is toxic to the cancer cells.

11. The method of claim 1, wherein the cancer cells comprise a mutant isocitrate dehydrogenase (mutIDH) that catalyzes a reaction of AKG to 2HG.

12. The method of claim 11, wherein the mutIDH comprises IDH1 (mutIDH1) or IDH2 (mutIDH2).

13. The method of claim 12, wherein the mutIDH comprises mutIDH1.

14. The method of claim 13, wherein a mutation in the mutIDH1 that catalyzes the reaction of AKG to 2HG comprises an amino acid substitution at R132.

15. The method of claim 13, wherein the mutIDH1 further comprises a mutation making the cancer cells resistant to a mutIDH1 inhibitor.

16. The method of claim 15, wherein the mutIDH1 inhibitor is an allosteric inhibitor.

17. The method of claim 15, wherein the mutIDH1 inhibitor does not bind to an active site of the mutIDH.

18. The method of claim 15, wherein the mutIDH1 inhibitor comprises Ivosidenib.

19. The method of claim 15, wherein the further mutation comprises an amino acid substitution at S280.

20. The method of claim 19, wherein the R132 and S280 amino acid substitutions are in the same allele of the mutIDH1 (cis).

21. The method of claim 12, wherein the mutIDH comprises mutIDH2.

22. The method of claim 21, wherein a mutation in the mutIDH2 that catalyzes the reaction of AKG to 2HG comprises an amino acid substitution at R140 or at R172.

23. The method of claim 21, wherein the mutIDH2 further comprises a mutation making the cancer cells resistant to a mutIDH2 inhibitor.

24. The method of claim 23, wherein the mutIDH2 inhibitor is an allosteric inhibitor.

25. The method of claim 23, wherein the mutIDH2 inhibitor does not bind to an active site of the mutIDH.

26. The method of claim 23, wherein the mutIDH2 inhibitor comprises Ensaidenib.

27. The method of claim 23, wherein the further mutation comprises an amino acid substitution at Q316 or I319.

28. The method of claim 27, wherein the amino acid substitutions R140, and Q316 or I319, are in a different allele of the mutIDH (trans).

29. The method of claim 1, wherein the cancer cells produce 2-hydroxyglutarate (2HG).

30. The method of claim 1, wherein treating the cancer comprises increasing an amount of 2HG in the cancer cells.

31. The method of claim 30, wherein the increased 2HG is toxic to the cancer cells.

32. The method of claim 1, wherein treating the cancer further comprises depleting AKG, reducing nicotinamide adenine dinucleotide (NADH), reducing nicotinamide adenine dinucleotide phosphate (NADPH) in cancer cells, or a combination thereof.

33. The method of claim 1, wherein hyperactivating the oncogenic activity comprises inducing activity of a mutant isocitrate dehydrogenase (mutIDH) in the cancer cells.

34. The method of claim 33, wherein hyperactivating the oncogenic activity of a mutIDH in the cancer cells comprises contacting the cancer cells with a molecule that hyperactivates the oncogenic activity of the mutIDH.

35. The method of claim 34, wherein the molecule increases an amount of 2HG in the cancer cells.

36. The method of claim 34, wherein the molecule depletes AKG, reduces an amount of nicotinamide adenine dinucleotide (NADH), reduces an amount nicotinamide adenine dinucleotide phosphate (NADPH) in cancer cells, or a combination thereof.

37. The method of claim 34, wherein the molecule activates the mutIDH.

38. The method of claim 34, wherein the molecule facilitates the mutIDH to modify its cofactor preference.

39. The method of claim 38, wherein modifying cofactor preference comprises mutIDH using NAD/NADH in addition to the preferred cofactors NADP/NADPH.

40. The method of claim 34, wherein the molecule comprises a small molecule.

41. The method of claim 34, wherein the molecule binds to the mutIDH in the cancer cells.

42. The method of claim 41, wherein the molecule binds to a site in the mutIDH different from a site where a mutIDH inhibitor binds.

43. The method of claim 41, wherein the molecule binds to a hydrophobic pocket at the dimer-interface of mutIDH, wherein the dimer-interface comprises the interface of mutIDH subunit A with mutIDH subunit B.

44. The method of claim 43, wherein the mutIDH dimer-interface comprises a mutIDH2 dimer-interface, and wherein the dimer-interface further comprises the interface of mutIDH2 subunit A with mutIDH2 subunit B.

45. The method of claim 43, wherein the mutIDH dimer-interface comprises a mutIDH1 dimer-interface, and wherein the dimer interface further comprises the interface of mutIDH1 subunit A with mutIDH1 subunit B.

46. The method of claim 43, wherein the molecule interacts with one or more amino acid residues selected from the group consisting of S200, K243, W244, P245, Y247, K282, W284, R288, M293, Q296, S301, G303, F304, and W306 in IDH2.

46A. The method of claim 43, wherein the molecule interacts with one or more homologous amino acid residues in IDH1.

46B. The method of claim 46A, wherein the one or more homologous amino acids in IDH1 are selected from the group consisting of K203, G204, W205, P206, Y208, K243, W245, R249, M254, Q257, S261, E262 and G264.

47. The method of claim 43, wherein the molecule repositions amino acid residue R288 of mutIDH2 subunit A and amino acid residue R353 of mutIDH2 subunit B to enhance π-π stacking across subunit A and subunit B to move the subunits proximally closer by about 0.1-0.9 Å, 0.2-0.8 Å or 0.3-0.6 Å.

48. The method of claim 43, wherein the molecule strengthens interactions across/within an interface of two mutIDH subunits.

49. The method of claim 40, wherein the molecule comprises clonixin, idebenone, dihydrogedunin, deoxygedunin, gossypetin, derivatives thereof and/or combinations thereof.

50. The method of claim 49, wherein the molecule comprises [2-(3-chloro-o-toluidino)nicotinic acid], 2-(2′-methyl-3′-chloro)-anilino-nicotinic acid, or a derivative thereof, and wherein the molecule induces mutIDH2 in cancer cells having an R140 mutation in the IDH2.

51. The method of claim 49, wherein the molecule comprises 7-ethyl-2,4-dimethylbenzo[b][1,8]naphthyridin-5(10H)-one, or a derivative thereof, and wherein the molecule induces mutIDH2 in cancer cells having an R172 mutation in the IDH2.

52. The method of claim 1 wherein the cancer is selected from the group consisting of acute myeloid leukemia, glioma, cholangiocarcinoma, chondrosarcoma and T cell lymphoma.

53. A method of treating a cancer containing a mutant isocitrate dehydrogenase (mutIDH), the method comprising administering to a subject a composition that increases activity of the mutIDH in cancer cells.

54. The method of claim 53, wherein the mutIDH catalyzes a reaction of α-ketoglutarate (αKG) to 2-hydroxyglutarate (2HG).

55. The method of claim 53, wherein the composition hyperactivates the mutIDH in the cancer cells.

56. The method of claim 53, wherein the composition induces the mutIDH to synthesize an increased amount of 2-hydroxyglutarate (2HG) in the cancer cells.

57. The method of claim 53, wherein the composition depletes AKG, reduces an amount of nicotinamide adenine dinucleotide (NADH), reduces an amount nicotinamide adenine dinucleotide phosphate (NADPH) in the cancer cells, or a combination thereof.

58. The method of claim 53, wherein the composition facilitates a cofactor preference for the mutIDH in the cancer cells.

59. The method of claim 58, wherein the composition increases consumption of NADPH, NADH and α-ketoglutarate in the cancer cells.

60. The method of claim 53, wherein the composition impairs growth and/or proliferation of the cancer cells, causes mitochondrial dysfunction in the cancer cells, cause metabolic dysfunction in the cancer cells, decreases amounts of nucleotide intermediates in the cancer cells, decreases amounts of amino acids in the cancer cells, or a combination thereof.

61. The method of claim 53, wherein the mutIDH comprises a mutation that facilitates the mutIDH to metabolize α-ketoglutarate (AKG) to 2-hydroxyglutarate (2HG) in the cancer cells.

62. The method of claim 61, wherein the mutIDH comprises a further mutation making the mutIDH resistant to a mutIDH inhibitor.

63. The method of claim 62, wherein the mutIDH comprises a mutIDH1 or a mutIDH2.

64. The method of claim 63, wherein the mutIDH comprises mutIDH1.

65. The method of claim 64, wherein the mutation facilitating metabolism of AKG to 2HG comprises an amino acid substitution at R132 or V71, or the mutation comprises SNP rs11554137.

66. The method of claim 64, wherein the mutation making mutIDH resistant to a mutIDH inhibitor comprises an amino acid substitution at S280.

67. The method of claim 63, wherein the mutIDH comprises mutIDH2.

68. The method of claim 67, wherein the mutation facilitating metabolism of AKG to 2HG comprises an amino acid substitution at R140 or R172.

69. The method of claim 67, wherein the mutation making mutIDH resistant to a mutIDH inhibitor comprises an amino acid substitution at Q316 or I319.

70. The method of claim 67, wherein the mutation enabling metabolism of AKG to 2HG and the mutation that makes the mutIDH1 resistant to the mutIDH inhibitor are in trans.

71. The method of claim 67, wherein the composition comprises clonixin, and derivatives thereof, and wherein the mutIDH2 in the cancer cells has an amino acid substitution at R140.

72. The method of claim 67, wherein the composition comprises EDBN, and derivatives thereof, and wherein the mutIDH2 in the cancer cells has an amino acid substitution at R172.

73. The method of claim 53, wherein the composition comprises a small molecule therapeutic.

74. The method of claim 73, wherein the composition comprises clonixin, or a derivative thereof.

75. The method of claim 73, wherein the composition comprises EDBN, a derivative thereof.

76. The method of claim 73, wherein the composition comprises clonixin, idebenone, dihydrogedunin, deoxygedunin, gossypetin, derivatives thereof, or combinations thereof.

77. A method for identifying a compound that interacts with cancer cells having a mutant isocitrate dehydrogenase (mutIDH), the method comprising screening for compounds that activate the mutIDH in a cell, wherein the mutIDH uses NADH to reduce α-ketoglutarate to 2-hydroxyglutarate.

78. A method for treating a cancer in a subject, the method comprising:
administering to the subject a vector comprising a gene encoding a hyperactivated mutant isocitrate dehydrogenase (mutIDH); and
expressing the mutIDH in cancer cells.

79. The method of claim 78, wherein the hyperactivated mutIDH produces 2-hydroxyglutarate (2HG).

80. The method of claim 78, wherein the hyperactivated mutIDH is resistant to a mutIDH inhibitor.

81. The method of claim 78, wherein the hyperactivated mutIDH comprises a mutIDH2.

82. The method of claim 81, wherein the mutIDH2 comprises an amino acid substitution in R140 or R172, and an amino acid substitution in Q316 or I319.

83. The method of claim 82, wherein the R140 or R172 amino acid substitution and the Q316 or I319 substitution are encoded in the same allele (in cis).

84. The method of claim 83, wherein a gene encoding mutIDH2 is introduced into the cancer cells using a retroviral or lentiviral vector.

85. A method for treating a cancer in a subject, the method comprising:
introducing a modification to an endogenous mutIDH gene to hyperactivate the gene in a cell of the cancer in the subject.

86. The method of claim 85, wherein the modification is introduced using CRISPR-based gene editing.

87. The method of any one of claims 1, 53, 78 or 85 used in combination with a chemotherapy.

87. The method of any one of claims 51, 53, 78 or 85 used in combination with a chemotherapy, wherein the chemotherapy targets nucleotide metabolism.

88. The method of anyone of claims 51, 53, 78 or 85 used in combination with a chemotherapy, wherein the chemotherapy comprises cytarabine.
